(19) United States
(12) Patent Application Publication
(10) Pub. No.: US 2023/0399400 A1
(43) Pub. Date: Dec. 14, 2023

(54) NOVEL ANTI-CD47 ANTIBODIES AND USES THEREOF

(71) Applicant: I-MAB BIOPHARMA CO., LTD., Shanghai (CN)

(72) Inventors: Zhengyi Wang, Shanghai (CN); Wei Cao, Shanghai (CN); Bingshi Guo, Shanghai (CN); Cong Xu, Rockville, MD (US)

(21) Appl. No.: 18/249,054

(22) PCT Filed: Oct. 14, 2021
(86) PCT No.: PCT/CN2021/123892

§ 371 Date: Apr. 13, 2023

(30) Foreign Application Priority Data
Oct. 14, 2020 (WO) PCT/CN2020/120869
Oct. 20, 2020 (WO) PCT/CN2020/122188

(51) Int. Cl. C07K 16/28; A61P 35/02; A61P 35/00

(52) U.S. Cl. CPC C07K 16/2803; A61P 35/02; A61P 35/00; C07K 2317/33; C07K 2317/24; C07K 2317/76; A61K 2039/545

(57) ABSTRACT

Provided herein are CD47 antibodies and immunologically active fragments thereof that have low immunogenicity in humans and cause low or no level of red blood cell depletion or hemagglutination. Also provided are as well as pharmaceutical compositions containing such antibodies or antibody fragments, as well as methods of treatment using such antibodies, e.g., as single agents or in combination with other therapeutic agent (s).

### CROSS REFERENCE TO RELATED APPLICATIONS

This application claims the priority benefit of International Application No. PCT/CN2020/120869, filed Oct. 14, 2020, and International Application No. PCT/CN2020/122188, filed Oct. 20, 2020, the contents of which are incorporated herein by reference in their entireties.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 233002000241SEQLIST.TXT, date recorded: Oct. 12, 2021, size: 31,455 bytes).

### FIELD OF THE INVENTION

The present application relates to anti-CD47 antibodies, methods of producing such antibodies, and use of such antibodies in the treatment of diseases and disorders associated with CD47.

### BACKGROUND OF THE INVENTION

CD47 (Cluster of Differentiation 47) was first identified as a tumor antigen on human ovarian cancer in the 1980s. Since then, CD47 has been found to be expressed on multiple human tumor types including acute myeloid leukemia (AML), chronic myeloid leukemia, acute lymphoblastic leukemia (ALL), non-Hodgkin's lymphoma (NHL), multiple myeloma (MM), bladder cancer, and other solid tumors. High levels of CD47 allow cancer cells to avoid phagocytosis despite having a higher level of calreticulin—the dominant pro-phagocytic signal.

Also known as integrin-associated protein (TAP), ovarian cancer antigen OA3, Rh-related antigen and MER6, CD47 is a multi-spanning transmembrane receptor belonging to the immunoglobulin superfamily. Its expression and activity have been implicated in a number of diseases and disorders. It is a broadly expressed transmembrane glycoprotein with a single Ig-like domain and five membrane spanning regions, which functions as a cellular ligand for SIRPα with binding mediated through the NH₂-terminal V-like domain of signal-regulatory-protein α (SIRPα). SIRPα is expressed primarily on myeloid cells, including macrophages, granulocytes, myeloid dendritic cells (DCs), mast cells, and their precursors, including hematopoietic stem cells.

Macrophages clear pathogens and damaged or aged cells from the blood stream via phagocytosis. Cell-surface CD47 interacts with its receptor on macrophages, SIRPα, to inhibit phagocytosis of normal, healthy cells. SIRPα inhibits the phagocytosis of host cells by macrophages, where the ligation of SIRPα on macrophages by CD47 expressed on the host target cell generates an inhibitory signal mediated by SHP-1 that negatively regulates phagocytosis.

In keeping with the role of CD47 to inhibit phagocytosis of normal cells, there is evidence that it is transiently up-regulated on hematopoietic stem cells (HSCs) and progenitors just prior to and during their migratory phase, and that the level of CD47 on these cells determines the probability that they are engulfed in vivo.

CD47 is also constitutively up-regulated on a number of cancers, including myeloid leukemias. Overexpression of CD47 on a myeloid leukemia line increases its pathogenicity by allowing it to evade phagocytosis. It has been concluded that CD47 up-regulation is an important mechanism for providing protection to normal HSCs during inflammation-mediated mobilization, and that leukemic progenitors co-opt this ability in order to evade macrophage killing.

Certain CD47 antibodies have been shown to restore phagocytosis and prevent atherosclerosis. See, e.g., Kojima et al., Nature, Vol. 36, 86-90 (Aug. 4, 2016). The present invention provides novel CD47 antibodies or immunologically active fragments thereof that have low immunogenicity in humans and cause low or no level of red blood cell depletion. As well known to a person skilled in the art, such antibodies may be interchangeably called “anti-CD47 antibodies.”

### SUMMARY OF THE INVENTION

Provided herein is an antibody or immunologically active fragment thereof that specifically binds to human CD47 (hCD47), comprising: (a) a heavy chain variable (V_{H}) domain that comprises (1) a glutamic acid residue (E) at its N-terminus; (2) a CDR-H1 comprising RAWMN (SEQ ID NO: 5); (3) a CDR-H2 comprising RIKRKTDGETTDYAAPVKG (SEQ ID NO: 6); (4) a CDR-H3 comprising SNRAFDI (SEQ ID NO: 7); and (5) a serine (S) at its C-terminus; and (b) a light chain variable (V_{L}) domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10).

In some embodiments, the N-terminal amino acid of the V_{H} domain corresponds to position H1 according to the Kabat numbering system, and the C-terminal amino acid of the V_{H} domain corresponds to position H113 according to the Kabat numbering system. In some embodiments, the N-terminal amino acid of the V_{H} domain corresponds to position H1 according to the Chothia numbering system, and the C-terminal amino acid of the V_{H} domain corresponds to position H113 according to the Chothia numbering system. In some embodiments, the N-terminal amino acid of the V_{H} domain corresponds to position H1 according to the IMGT numbering system, and the C-terminal amino acid of the V_{H} domain corresponds to position H128 according to the IMGT numbering system. In some embodiments, the N-terminal amino acid of the V_{H} domain corresponds to amino acid 1 of SEQ ID NO: 1, and the C-terminal amino acid of the V_{H} domain corresponds to amino acid 118 of SEQ ID NO: 1. In some embodiments, the V_{H} comprises an amino acid sequence that has at least 95% identity to SEQ ID NO: 1, and the V_{L} comprises an amino acid sequence that has at least 95% identity to SEQ ID NO: 2. In some embodiments, the V_{H} comprises SEQ ID NO: 1, and the V_{L} comprises SEQ ID NO: 2.

In some embodiments, the anti-CD47 antibody or immunologically active fragment thereof comprises an Fc domain. In some embodiments, the Fc domain is a human IgG Fc domain. In some embodiments, the human IgG Fc domain is an IgG1, IgG2, IgG3, or IgG4 Fc domain. In some embodiments, the anti-CD47 antibody is a full length antibody. In some embodiments, the full length anti-CD47 antibody comprises a heavy chain that comprises SEQ ID NO: 3 or SEQ ID NO: 55 and a light chain that comprises SEQ ID NO: 4. In some embodiments, the immunologically active fragment of the anti-CD47 antibody is a Fab, a Fab′, a F(ab)′2, a Fab′-SH, a single-chain Fv (scFv), an Fv fragment, or a linear antibody. In some embodiments, the anti-CD47 antibody or immunologically active fragment thereof is a monoclonal antibody or fragment thereof. In some embodiments, the anti-CD47 antibody or immunologically active fragment thereof is chimeric or humanized.

In some embodiments, the anti-CD47 antibody or immunologically active fragment thereof binds to hCD47 expressed on the surface of a cancer cell. In some embodiments, the cancer cell is a SK-OV-3 cell, a Toledo cell, a K562 cell, a HCC827 cell, a Jurkat cell, a U937 cell, a TF-1 cell, a Raji cell, a SU-DHL-4 cell, a MDA-MB-231 cell, an A375 cell, or a SK-MES-1 cell. In some embodiments, the cancer cell is a solid tumor cancer. In some embodiments, the solid tumor cancer is lung cancer, ovarian cancer, colorectal cancer, pancreatic cancer, sarcoma cancer, head and neck cancer, gastric cancer, renal cancer, or skin cancer. In some embodiments, the cancer cell is a hematological cancer. In some embodiments, the hematological cancer is non-Hodgkin lymphoma.

In some embodiments, the anti-CD47 antibody or immunologically active fragment thereof does not bind to hCD47 expressed on the surface of a blood cell. In some embodiments, the blood cell is an erythrocyte. In some embodiments, the binding of the anti-CD47 antibody or immunologically active fragment thereof to hCD47 prevents interaction of the hCD47 with signal-regulatory-protein α (SIRPα). In some embodiments, the SIRPα is human SIRPα (hSIRPα). In some embodiments, the binding of the anti-CD47 antibody or immunologically active fragment thereof to hCD47 expressed on the surface of a cancer cell promotes macrophage-mediated phagocytosis of the cancer cell. In some embodiments, the cancer cell is a SK-OV-3 cell, a Toledo cell, a K562 cell, a HCC827 cell, a Jurkat cell, a U937 cell, a TF-1 cell, a Raji cell, a SU-DHL-4 cell, a MDA-MB-231 cell, an A375 cell, or a SK-MES-1 cell. In some embodiments, the cancer cell is a solid tumor cancer. In some embodiments, the solid tumor cancer is lung cancer, ovarian cancer, colorectal cancer, pancreatic cancer, sarcoma cancer, head and neck cancer, gastric cancer, renal cancer, or skin cancer. In some embodiments, the cancer cell is a hematological cancer. In some embodiments, the hematological cancer is non-Hodgkin lymphoma. In some embodiments, administration of the anti-CD47 antibody or immunologically active fragment thereof to a subject does not cause a significant level of hemagglutination in the subject or depletion of the subject's red blood cells.

Provided herein are nucleic acids encoding an anti-CD47 antibody or immunologically active fragment thereof described herein. Also provided are vectors comprising such nucleic acids. Additionally, provided are host cells comprising the nucleic acids and/or the vectors described herein. In some embodiments, the host cell is a mammalian cell. In some embodiments, the mammalian cell is a Chinese hamster ovary (CHO) cell. In some embodiments, the CHO cell is a CHO-K1 cell. In a related aspect, provided are methods of producing an anti-CD47 antibody or immunologically active fragment thereof, comprising: a) culturing the host cell described herein under conditions effective to cause expression of the anti-CD47 antibody or antigen-binding fragment thereof; and b) recovering the anti-CD47 antibody or immunologically active fragment thereof expressed by the host cell.

Provided is pharmaceutical composition comprising an anti-CD47 antibody or immunologically active fragment thereof and pharmaceutically acceptable carrier.

Provided are methods of treating cancer in a subject, comprising administering an effective amount of an anti-CD47 antibody to the subject, wherein the anti-CD47 antibody comprises (a) a heavy chain variable domain (V_{H}) that comprises (1) a CDR-H1 comprising RAWMN (SEQ ID NO: 5); (2) a CDR-H2 comprising RIKRKTDGETTDYAAPVKG (SEQ ID NO: 6); and (3) a CDR-H3 comprising SNRAFDI (SEQ ID NO: 7); (b) a light chain variable (V_{L}) domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10).

In some embodiments, the cancer is solid tumor. In some embodiments, the solid tumor is a lung tumor, an ovarian tumor, a colorectal tumor, a pancreatic tumor, a sarcoma tumor, a head and neck tumor, a gastric tumor, a renal tumor, or a skin tumor. In some embodiments, the solid tumor is relapsed and/or refractory solid tumor.

In some embodiments, the cancer is non-Hodgkin lymphoma (NHL), and the method further comprises administering an effective amount of rituximab to the subject. In some embodiments, the NHL is follicular lymphoma (FL), diffuse large B-cell lymphoma (DLBCL), or mantle cell lymphoma (MCL). In some embodiments, the NHL is relapsed/refractory NHL. In some embodiments, the subject has undergone at least one prior treatment for NHL. In some embodiments, the subject has undergone between 2 and 10 prior therapies for NHL. In some embodiments, the subject has undergone prior treatment for NHL with an agent that targets CD20. In some embodiments, the subject progressed during or after the prior therapy with an agent that targets CD20.

In some embodiments of any of the methods of treatment provided herein, the anti-CD47 antibody comprises a human IgG4 constant region or a variant thereof comprising an S233P mutation (wherein numbering is according to the EU index). In some embodiments, the anti-CD47 antibody is administered to the subject at a dose of 10 mg/kg. In some embodiments, the anti-CD47 antibody is administered to the subject at a dose of 20 mg/kg. In some embodiments, the anti-CD47 antibody is administered to the subject at a dose of 30 mg/kg. In some embodiments, the anti-CD47 antibody is administered to the subject once every week (qw). In some embodiments, wherein the anti-CD47 antibody is administered to the subject via intravenous (IV) in fusion. In some embodiments of the methods of treating NHL, the rituximab is administered at a dose of 375 mg/m² once a week (qw) for a first five weeks and at a dose of 375 mg/m² once every 4 weeks (q4w) following the first five weeks.

In some embodiments of the methods of treatment described herein, the subject does not experience significant hematological toxicity due to the treatment with the anti-CD47 antibody. In some embodiments, the subject does not experience any hematological toxicity due to treatment with the anti-CD47 antibody. In some embodiments, the hematological toxicity comprises anemia, cytopenia, and/or hemagglutination. In some embodiments, the V_{H} of the anti-CD47 antibody comprises SEQ ID NO: 1, and the V_{L} of the anti-CD47 antibody comprises SEQ ID NO: 2. In some embodiments, the heavy chain of the anti-CD47 antibody comprises SEQ ID NO: 3 or SEQ ID NO: 55 and the light chain of the anti-CD47 antibody comprises SEQ ID NO: 4.

Also provided are kits for treating cancer comprising an anti-CD47 antibody or pharmaceutical composition described herein. In some embodiments, the kit is for use according to a method of treatment provided herein.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTIONS OF THE DRAWINGS

FIG. 1 shows dose-dependent response of anti-CD47 antibodies B2B, 5F9, and 2A1 binding to monomeric CD47-ECD (extracellular domain).

FIG. 2 shows dose-dependent response of anti-CD47 antibodies B2B, 5F9, and 2A1 blocking the binding of CD47 to SIRPα.

FIG. 3 shows dose-dependent response of anti-CD47 antibodies B2B, 5F9, and 2A1 binding to CD47⁺ Raji cells.

FIG. 4 shows binding of tumor cells of CD47 antibodies as measured by surface plasmon resonance (BiaCore) analysis.

FIG. 5 shows that the binding of anti-CD47 antibody B2B to CD47 expressed on Raji cells promotes phagocytosis of Raji cells by human macrophages.

FIG. 6 shows that binding of anti-CD47 antibody B2B to CD47 expressed on various human tumor cell lines promotes phagocytosis of the tumor cells by human macrophages.

FIG. 7 shows binding of acute myeloid leukemia (AML) cells by CD47 antibodies.

FIG. 8 shows phagocytosis of acute myeloid leukemia (AML) cells by CD47 antibodies.

FIGS. 9A and 9B show that the anti-CD47 antibody B2B resulted in minimal binding to red blood cells (RBCs) and no RBC agglutination. Specifically, FIG. 9A shows minimal RBC binding by anti-CD47 antibody B2B and FIG. 9B shows no RBC agglutination by the anti-CD47 antibody B2B.

FIGS. 10A-10B show that the anti-CD47 antibody B2B did not induce significant hematologic changes in cynomolgus monkey following administration. FIG. 10A illustrates that single dose treatment of B2B showed a minimal influence on the level of RBCs and hemoglobin as compared to the treatment of 5F9. FIG. 10B illustrates that repeated treatments of B2B with different dosage did not significantly affect the RBCs in both male and female cynomolgus monkeys as compared to vehicle control.

FIG. 11 shows the in vivo efficacy of treatment with the CD47 antibody B2B in a luciferase-Raji xenograft model in mice.

FIG. 12A shows the time course of hemoglobin counts, respectively, following administration of the anti-CD47 antibody B2B. FIG. 12B shows the time course of reticulocyte counts, respectively, following administration of the anti-CD47 antibody B2B.

FIGS. 13A and 13B show the serum pharmacokinetics (PK) of the antiCD47 antibody B2B Q1W following a single dose and multiple doses. Specifically, FIG. 13A shows the serum PK of the anti-CD47 antibody B2B Q1W following a single dose, and FIG. 13B shows the serum PK of the anti-CD47 antibody B2B Q1W following multiple doses.

FIG. 14 shows CD47 receptor occupancy (RO) on peripheral T cells following weekly administration of the CD47 antibody B2B at various concentrations.

FIG. 15 shows the amino acid sequences of anti-CD47 antibodies B2B and C3C.

FIG. 16A shows the titer of B2B antibody and C3C antibody produced by Acti-pro medium on Day 10. FIG. 16B shows the end titer of B2B antibody and C3C antibody produced by Acti-pro medium.

FIG. 17 provides the study design for the Phase I study described in Example 21.

FIG. 18A shows a time course of hemoglobin and reticulocyte levels of all 20 patients who participated in the Phase I study described in Example 21. FIG. 18B shows a time course of hemoglobin and reticulocyte levels of in patients receiving the highest dose of anti-CD47 antibody (30 mg/kg) in the Phase I study described in Example 21

FIG. 19A shows serum pharmacokinetics of anti-CD47 antibody lemzoparlimab in patients following a single dose. FIG. 19B shows serum pharmacokinetics of anti-CD47 antibody lemzoparlimab qw in patients following multiple doses.

FIG. 20 shows % receptor occupancy of CD47 by anti-CD47 antibody lemzoparlimab on peripheral T cells in patients receiving weekly antibody administrations at 20 or 30 mg/kg

FIG. 21 shows responding hepatic metastases in a melanoma patient from Example 21 who received treatment with the anti-CD47 antibody.

### DETAILED DESCRIPTION OF THE INVENTION

### Definitions

Before describing the embodiments in detail, it is to be understood that the present disclosure is not limited to particular compositions or biological systems, which can, of course, vary. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the present disclosure include “comprising,” “consisting,” and “consisting essentially of” aspects and embodiments.

The term “CD47” (which is also known as Integrin Associated Protein (TAP), Antigenic Surface Determinant Protein OA3, OA3, CD47 Antigen, Rh-Related Antigen, Integrin-Associated Signal Transducer, Antigen Identified By Monoclonal Antibody 1D8, CD47 glycoprotein) preferably refers to human CD47 and, in particular, to a protein comprising the amino acid sequence

(SEQ ID NO: 14
MWPLVAALLL GSACCGSAQL LENKTKSVEF TFCNDTVVIP
CFVINMEAQN TTEVYVKWKF KGRDIYTFDG ALNKSTVPTD
FSSAKIEVSQ LLKGDASLKM DKSDAVSHTG NYTCEVTELT
REGETIIELK YRVVSWFSPN ENILIVIFPI FAILLFWGQF
GIKTLKYRSG GMDEKTIALL VAGLVITVIV IVGAILFVPG
EYSLKNATGL GLIVTSTGIL ILLHYYVEST AIGLTSEVIA
ILVIQVIAYI LAVVGLSLCI AACIPMHGPL LISGLSILAL
AQLLGLVYMK FVASNQKTIQ PPRKAVEEPL NAFKESKGMM NDE

or a variant of said amino acid sequence. The term “CD47” also refers to any post translationally modified variants and conformation variants.

As used herein, the term “antibody” is used in the broadest sense and specifically covers intact antibodies (e.g., full length antibodies), antibody fragments (including without limitation Fab, F(ab′)2, Fab′-SH, Fv, diabodies, scFv, scFv-Fc, single domain antibodies, single heavy chain antibodies, and single light chain antibodies), monoclonal antibodies, and polyclonal antibodies, so long as they exhibit the desired biological activity (e.g., epitope binding). “Antibodies” (or “Abs”) and “immunoglobulins” (or “Igs”) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

As used herein, the term “isolated” antibody may refer to an antibody that is substantially free of other cellular material. In one embodiment, an isolated antibody is substantially free of other proteins from the same species. In another embodiment, an isolated antibody is expressed by a cell from a different species and is substantially free of other proteins from the different species. In some embodiments, an “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. An antibody may be rendered substantially free of naturally associated components (or components associated with the cellular expression system used to produce the antibody) by isolation, using protein purification techniques well known in the art. In some embodiments, the antibody will be purified (1) to greater than 75% by weight of antibody as determined by the Lowry method, and most preferably more than 80%, 90%, 95% or 99% by weight, or (2) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the term “epitope” means any antigenic determinant on an antigen to which the paratope of an antibody binds. Epitopic determinants usually consist of chemically active surface groupings of molecules such as amino acids or sugar side chains and usually have specific three-dimensional structural characteristics, as well as specific charge characteristics.

As used herein, the term “native antibodies and immunoglobulins” are usually heterotetrameric glycoproteins of about 150,000 daltons, composed of two identical light (L) chains and two identical heavy (H) chains. Each light chain is linked to a heavy chain by one covalent disulfide bond (also termed a “VH/VL pair”), while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light- and heavy-chain variable domains. See, e.g., Chothia et al., J. Mol. Biol., 186:651 (1985); Novotny and Haber, Proc. Natl. Acad. Sci. U.S.A., 82:4592 (1985).

As used herein, the term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions both in the light-chain and the heavy-chain variable domains. The more highly conserved portions of variable domains are called the framework (FR). The variable domains of native heavy and light chains each comprise four FR regions, largely adopting a β-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. See, e.g., Kabat et al., Sequences of Proteins of Immunological Interest, Fifth Edition, National Institute of Health, Bethesda, Md. (1991). The constant domains are not involved directly in binding an antibody to an antigen, but exhibit various effector functions, such as participation of the antibody in antibody-dependent cellular toxicity. Variable region sequences of interest include the humanized variable region sequences for CD47 antibodies described in detail elsewhere herein.

The term “hypervariable region (HVR)” or “complementarity determining region (CDR)” may refer to the subregions of the VH and VL domains characterized by enhanced sequence variability and/or formation of defined loops. These include three CDRs in the VH domain (H1, H2, and H3) and three CDRs in the VL domain (L1, L2, and L3). H3 is believed to be critical in imparting fine binding specificity, with L3 and H3 showing the highest level of diversity. See Johnson and Wu, in Methods in Molecular Biology 248:1-25 (Lo, ed., Human Press, Totowa, N.J., 2003).

A number of CDR/HVR delineations are known. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs/CDRs are noted below. “Framework” or “FR” residues are those variable domain residues other than the HVR/CDR residues.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |

(Kabat Numbering)

| | | | | |
|---|---|---|---|---|
| H1 | H31-H35 | H26-H35 | H26-H32 | H30-H35 |

(Chothia Numbering)

| | | | | |
|---|---|---|---|---|
| H2 | H50-H65 | H50-H58 | H53-H55 | H47-H58 |
| H3 | H95-H102 | H95-H102 | H96-H101 | H93-H101 |

“Extended” HVRs are also known: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH (Kabat numbering).

“Numbering according to Kabat” may refer to the numbering system used for heavy chain variable domains or light chain variable domains of the compilation of antibodies in Kabat et al., supra. The actual linear amino acid sequence may contain fewer or additional amino acids corresponding to a shortening of, or insertion into, a FR or HVR of the variable domain. The Kabat numbering of residues may be determined for a given antibody by alignment at regions of homology of the sequence of the antibody with a “standard” Kabat numbered sequence. Typically, the Kabat numbering is used when referring to a residue in the variable domains (approximately residues 1-107 of the light chain and residues 1-113 of the heavy chain), whereas the EU numbering system or index (e.g., the EU index as in Kabat, numbering according to EU IgG1) is generally used when referring to a residue in the heavy chain constant region.

As used herein, a “monoclonal” antibody refers to an antibody obtained from a population of substantially homogeneous antibodies, e.g., substantially identical but allowing for minor levels of background mutations and/or modifications. “Monoclonal” denotes the substantially homogeneous character of antibodies, and does not require production of the antibody by any particular method. In some embodiments, a monoclonal antibody is selected by its HVR, VH, and/or VL sequences and/or binding properties, e.g., selected from a pool of clones (e.g., recombinant, hybridoma, or phage-derived). A monoclonal antibody may be engineered to include one or more mutations, e.g., to affect binding affinity or other properties of the antibody, create a humanized or chimeric antibody, improve antibody production and/or homogeneity, engineer a multispecific antibody, resultant antibodies of which are still considered to be monoclonal in nature. A population of monoclonal antibodies may be distinguished from polyclonal antibodies as the individual monoclonal antibodies of the population recognize the same antigenic site. A variety of techniques for production of monoclonal antibodies are known; see, e.g., the hybridoma method (e.g., Kohler and Milstein, Nature, 256:495-97 (1975); Hongo et al., Hybridoma, 14 (3): 253-260 (1995), Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,806; 5,569,825; 5,625,126; 5,633,425; and U.S. Pat. No. 5,661,016; Marks et al., Bio/Technology 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg and Huszar, Intern. Rev. Immunol. 13: 65-93 (1995).

“Chimeric” antibodies may refer to an antibody with one portion of the heavy and/or light chain from a particular isotype, class, or organism and another portion from another isotype, class, or organism. In some embodiments, the variable region will be from one source or organism, and the constant region will be from another.

“Humanized antibodies” may refer to antibodies with predominantly human sequence and a minimal amount of non-human (e.g., mouse or chicken) sequence. In some embodiments, a humanized antibody has one or more HVR sequences (bearing a binding specificity of interest) from an antibody derived from a non-human (e.g., mouse or chicken) organism grafted onto a human recipient antibody framework (FR). In some embodiments, non-human residues are further grafted onto the human framework (not present in either source or recipient antibodies), e.g., to improve antibody properties. In general, a humanized antibody will comprise substantially all of at least one, and typically two, variable domains, in which all or substantially all of the hypervariable loops correspond to those of a non-human immunoglobulin, and all or substantially all of the FRs are those of a human immunoglobulin sequence. The humanized antibody optionally will also comprise at least a portion of an immunoglobulin constant region (Fc), typically that of a human immunoglobulin. See Jones et al., Nature 321:522-525 (1986); Riechmann et al., Nature 332:323-329 (1988); and Presta, Curr. Op. Struct. Biol. 2:593-596 (1992).

A “human” antibody may refer to an antibody having an amino acid sequence which corresponds to that of an antibody produced by a human and/or has been made using any of the techniques for making human antibodies as disclosed herein. Human antibodies can be produced using various techniques known in the art, including phage-display libraries. Hoogenboom and Winter, J. Mol. Biol., 227:381 (1991); Marks et al., J. Mol. Biol., 222:581 (1991); preparation of human monoclonal antibodies as described in Cole et al., Monoclonal Antibodies and Cancer Therapy, Alan R. Liss, p. 77 (1985); Boerner et al., J. Immunol., 147(1):86-95 (1991); and by administering the antigen to a transgenic animal that has been modified to produce such antibodies in response to antigenic challenge, but whose endogenous loci have been disabled, e.g., immunized xenomice (see, e.g., U.S. Pat. Nos. 6,075,181 and 6,150,584 regarding XENOMOUSE™ technology) or chickens with human immunoglobulin sequence(s) (see, e.g., WO2012162422, WO2011019844, and WO2013059159).

There are five major classes of immunoglobulins: IgA, IgD, IgE, IgG, and IgM, and several of these can be further divided into subclasses (isotypes), e.g., IgG1, IgG2, IgG3, IgG4, IgA1, IgA2. The heavy-chain constant domains that correspond to the different classes of immunoglobulins are called a, 6, £, γ, and respectively. The subunit structures and three-dimensional configurations of different classes of immunoglobulins are well known.

As used herein, the term “antibody fragment”, and all grammatical variants thereof, are defined as a portion of an intact antibody comprising the antigen binding site or variable region of the intact antibody which, in certain instances, is free of the constant heavy chain domains (i.e. CH2, CH3, and/or CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules, (2) single chain polypeptides containing only one light chain variable domain, or a fragment thereof that contains the three CDRs of the light chain variable domain, without an associated heavy chain moiety, and (3) single chain polypeptides containing only one heavy chain variable region, or a fragment thereof containing the three CDRs of the heavy chain variable region, without an associated light chain moiety; and multi-specific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen. “Fv” is the minimum antibody fragment which contains a complete antigen-recognition and -binding site. In a two-chain Fv species, this region consists of a dimer of one heavy- and one light-chain variable domain in tight, non-covalent association. In a single-chain Fv species (scFv), one heavy- and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site. See, e.g., Pluckthun, in The Pharmacology of Monoclonal Antibodies, Vol. 113, Rosenburg and Moore eds., Springer-Verlag, New York, pp. 269-315 (1994).

The Fab fragment also contains the constant domain of the light chain and the first constant domain (CHO of the heavy chain. Fab′ fragments differ from Fab fragments by the addition of a few residues at the carboxy terminus of the heavy chain CH₁ domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

As used herein, the term “pharmaceutically acceptable carrier or excipient” refers to a carrier or an excipient that is useful for preparing a pharmaceutical composition or formulation that is generally safe, non-toxic, and neither biologically nor otherwise undesirable. A carrier or excipient employed is typically one suitable for administration to human subjects or other mammals. In making the compositions, the active ingredient is usually mixed with, diluted by, or enclosed with a carrier or excipient. When the carrier or excipient serves as a diluent, it can be a solid, semi-solid, or liquid material, which acts as a vehicle, carrier, or medium for the active ingredient of the antibody.

As used herein, the term “monoclonal antibody” (mAb) refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Each mAb is directed against a single determinant on the antigen. In addition to their specificity, the monoclonal antibodies are advantageous in that they can be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made in an immortalized B cell or hybridoma thereof, or may be made by recombinant DNA methods.

The monoclonal antibodies herein include hybrid and recombinant antibodies produced by splicing a variable (including hypervariable) domain of an CD47 antibody with a constant domain (e.g. “humanized” antibodies), or a light chain with a heavy chain, or a chain from one species with a chain from another species, or fusions with heterologous proteins, regardless of species of origin or immunoglobulin class or subclass designation, as well as antibody fragments (e.g., Fab, F(ab′)₂, and Fv), so long as they exhibit the desired biological activity.

The monoclonal antibodies herein specifically include chimeric antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

As used herein, the term “epitope tagged” refers to a CD47 antibody fused to an “epitope tag”. The epitope tag polypeptide has enough residues to provide an epitope against which an antibody can be made, yet is short enough such that it does not interfere with activity of the CD47 antibody. The epitope tag preferably is sufficiently unique so that the antibody specific for the epitope does not substantially cross-react with other epitopes. Suitable tag polypeptides generally have at least 6 amino acid residues and usually between about 8-50 amino acid residues (preferably between about 9-30 residues). Examples include the c-myc tag and the 8F9, 3C7, 6E10, G4, B7 and 9E10 antibodies thereto (see, e.g., Evan et al., Mol. Cell. Biol., 5(12):3610-3616 (1985)); and the Herpes Simplex virus glycoprotein D (gD) tag and its antibody (see, e.g., Paborsky et al., Protein Engineering, 3(6):547-553 (1990)).

As used herein, the term “label” refers to a detectable compound or composition which is conjugated directly or indirectly to the antibody. The label may itself be detectable by itself (e.g., radioisotope labels or fluorescent labels) or, in the case of an enzymatic label, may catalyze chemical alteration of a substrate compound or composition which is detectable.

As used herein, the term “treatment” refers to clinical intervention designed to alter the natural course of the individual or cell being treated during the course of clinical pathology. Desirable effects of treatment include decreasing the rate of disease progression, ameliorating or palliating the disease state, and remission or improved prognosis. For example, an individual is successfully “treated” if one or more symptoms associated with cancer are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of individuals.

As used herein, “delaying progression of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

Exemplary cancers include, but are not limited to, ovarian cancer, colon cancer, breast cancer, lung cancer, head and neck cancer, bladder cancer, colorectal cancer, pancreatic cancer, non-Hodgkin's lymphoma, acute lymphocytic leukemia, chronic lymphocytic leukemia, acute myeloid leukemia, chronic myelogenous leukemia, multiple myeloma, melanoma, leiomyoma, leiomyosarcoma, glioma, glioblastoma, myelomas, monocytic leukemias, B-cell derived leukemias, T-cell derived leukemias, B-cell derived lymphomas, T-cell derived lymphomas, and solid tumors. The fibrotic disease can be, e.g., myocardial infarction, angina, osteoarthritis, pulmonary fibrosis, asthma, cystic fibrosis, bronchitis, or asthma.

As used herein, the terms “prevent,” “preventing,” and “prevention” are meant to include a method of delaying and/or precluding the onset of a condition, disorder, or disease, and/or its attendant symptoms; barring a subject from acquiring a condition, disorder, or disease; or reducing a subject's risk of acquiring a condition, disorder, or disease.

As used herein, the term “subject” for purposes of treatment refers to any animal classified as a mammal, including humans, domestic and farm animals, and zoo, sports, or pet animals, such as dogs, horses, cats, cows, etc. Preferably, the mammal is human.

All references cited herein, including patent applications and publications, are hereby incorporated by reference in their entirety.

### Overview

Provided herein are novel anti-CD47 antibodies that prevent human CD47 (hCD47) from interacting with SIRPα (e.g., human SIRPα or “hSIRPα”). In some embodiments the anti-CD47 antibody promotes macrophage-mediated phagocytosis of a CD47-expressing cell (e.g., an hCD47-expressing cell, such as a cancer cell). One of the challenges in the development of therapeutic anti-CD47 antibodies has been on-target, off-tissue toxicity. Red blood cells also express CD47 to prevent their destruction by the immune system, and CD47 therapies have run into dose-limiting hematological toxicities.

The anti-CD47 antibodies described herein are highly differentiated from many other known anti-CD47 antibodies, in that the anti-CD47 antibodies described herein bind a unique epitope on CD47 that is shielded by glycosylation on red blood cells, resulting in increased binding to CD47 expressed on the surface of, e.g., tumor cells. Advantageously, the anti-CD47 antibodies provided herein do not cause (e.g., do not cause a significant or noticeable level) of hemagglutination or depletion of red blood cells following administration to a subject (e.g., a human or a non-human primate).

Moreover, Applicant unexpectedly found that higher antibody titers were obtained from host cells expressing nucleic acids encoding the CD47 antibodies described herein, than from host cells cultured under the same conditions, but expressing nucleic acids encoding a highly similar anti-CD47 antibody. Specifically, the anti-CD47 antibodies provided herein comprise a VH domain that comprises a glutamic acid (E) at its N-terminus and a serine (S) at its C-terminus. Such antibodies can be produced by a mammalian host cell (e.g., a CHO cell, such as a CHO-K1 cell) in higher yields than an anti-CD47 antibody comprising a VH domain that comprises the same CDRs, but with an amino acid other than a glutamic acid (E) at its N-terminus and an amino acid other than a serine (S) at its C-terminus.

### Anti-CD47 Antibodies

An anti-CD47 antibody (or an immunologically active fragment thereof) is an antibody that binds to CD47 (e.g., human CD47 or “hCD47”) with sufficient affinity and specificity. As used herein, an “immunologically active fragment” of an antibody refers to an antigen-binding fragment of said antibody. The terms “immunologically active fragment” and “antigen-binding fragment” are used interchangeably herein. For example, an anti-CD47 antibody provided herein (or an immunologically active fragment thereof) may be used as a therapeutic agent in targeting and interfering with diseases or conditions associated with aberrant/abnormal CD47 expression and/or activity. In some embodiments, the anti-CD47 antibody is a chimeric (such as humanized) monoclonal antibody. In some embodiments, the anti-CD47 antibody comprises a heavy chain variable domain (VH), and/or a light chain variable domain (VL) of described herein below.

In some embodiments, the anti-CD47 antibody (or immunologically active fragment thereof) comprises a (a) VH domain that comprises (1) a glutamic acid residue (E) at its N-terminus; (2) a CDR-H1 comprising RAWMN (SEQ ID NO: 5); (3) a CDR-H2 comprising RIKRKTDGETTDYAAPVKG (SEQ ID NO: 6); (4) a CDR-H3 comprising SNRAFDI (SEQ ID NO: 7); and (5) a serine (S) at its C-terminus and (b) a VL domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10). In some embodiments, the CDR sequences are defined according to Kabat (see, e.g., (Kabat et al., Sequences of Proteins of Immunological Interest, 5th Ed. Public Health Service, National Institutes of Health, Bethesda, Md. (1991)). In some embodiments, the anti-CD47 antibody (or immunologically active fragment thereof) comprises a (a) VH domain that comprises (1) a glutamic acid residue (E) at its N-terminus; (2) a CDR-H1 comprising GLTFERA (SEQ ID NO: 21); (3) a CDR-H2 comprising KRKTDGET (SEQ ID NO: 22); (4) a CDR-H3 comprising SNRAFDI (SEQ ID NO: 7); and (5) a serine (S) at its C-terminus and (b) a VL domain that comprises (1) a CDR-L1 comprising KSSQSVLYAGNNRNYLA (SEQ ID NO: 24); (2) a CDR-L2 comprising QASTRAS (SEQ ID NO: 25); and (3) a CDR-L3 comprising QQYYTPPLA (SEQ ID NO: 26). In some embodiments, the CDR sequences are defined according to the Chothia numbering scheme (see, e.g., Chothia and Lesk (1986) EMBO J. 5(4):823-6 and Al-Lazikani et al., (1997) JMB 273: 927-948). In some embodiments, the anti-CD47 antibody (or immunologically active fragment thereof) comprises a (a) VH domain that comprises (1) a glutamic acid residue (E) at its N-terminus; (2) a CDR-H1 comprising GLTFERAW (SEQ ID NO: 27); (3) a CDR-H2 comprising IKRKTDGETT (SEQ ID NO: 28); (4) a CDR-H3 comprising AGSNRAFDI (SEQ ID NO: 29); and (5) a serine (S) at its C-terminus and (b) a VL domain that comprises (1) a CDR-L1 comprising QSVLYAGNNRNY (SEQ ID NO: 30); (2) a CDR-L2 comprising QA (SEQ ID NO: 31); and (3) a CDR-L3 comprising QQYYTPPLA (SEQ ID NO: 32). In some embodiments, the CDR sequences are defined according to the IMGT numbering scheme (see, e.g., Lefranc MP. (2013) IMGT Unique Numbering. In: Dubitzky W., Wolkenhauer O., Cho K H., Yokota H. (eds) Encyclopedia of Systems Biology. Springer, New York, NY; https://doi.org/10.1007/978-1-4419-9863-7_127). In some embodiments, the anti-CD47 antibody (or immunologically active fragment thereof) comprises a (a) VH domain that comprises (1) a glutamic acid residue (E) at its N-terminus; (2) a CDR-H1 comprising GLTFERAWMN (SEQ ID NO: 33); (3) a CDR-H2 comprising RIKRKTDGETTD (SEQ ID NO: 34); (4) a CDR-H3 comprising SNRAFDI (SEQ ID NO: 35); and (5) a serine (S) at its C-terminus and (b) a VL domain that comprises (1) a CDR-L1 comprising KSSQSVLYAGNNRNYLA (SEQ ID NO: 36); (2) a CDR-L2 comprising QASTRAS (SEQ ID NO: 37); and (3) a CDR-L3 comprising QQYYTPPLA (SEQ ID NO: 38). In some embodiments, the CDR sequences are defined according to the AbM numbering scheme (see, e.g., Abhinandan R. K., Martin A. C. Analysis and improvements to Kabat and structurally correct numbering of antibody variable domains. Mol. Immunol. 2008; 45:3832-3839. doi: 10.1016/j.molimm.2008.05 022).

In some embodiments, the anti-CD47 antibody (or immunologically active fragment thereof) comprises a (a) VH domain that comprises (1) a glutamic acid residue (E) at its N-terminus; (2) a CDR-H1 comprising ERAWMN (SEQ ID NO: 39); (3) a CDR-H2 comprising WVGRIKRKTDGETTD (SEQ ID NO: 40); (4) a CDR-H3 comprising AGSNRAFD (SEQ ID NO: 41); and (5) a serine (S) at its C-terminus and (b) a VL domain that comprises (1) a CDR-L1 comprising LYAGNNRNYLAWY (SEQ ID NO: 42); (2) a CDR-L2 comprising LLINQASTRA (SEQ ID NO: 43); and (3) a CDR-L3 comprising QQYYTPPL (SEQ ID NO: 44). In some embodiments, the CDR sequences are defined according to the Contact numbering scheme (see, e.g., McCallum et al. (1996) J Mol Biol. 262(5):732-45; doi: 10.1006/jmbi.1996.0548).

For ease of reference, the amino acid sequences of SEQ ID NOs: 5-10 and are provided in Table A below.

TABLE A

| | | |
|---|---|---|
| RAWMN | RIKRKTDGETTDYAAPVKG | SNRAFDI |
| (SEQ ID NO: 5) | (SEQ ID NO: 6) | (SEQ ID NO: 7) |
| KSSQSVLYAGNNRNYLA | QASTRAS | QQYYTPPLA |
| (SEQ ID NO: 8) | (SEQ ID NO: 9) | (SEQ ID NO: 10) |
| GLTFERA | KRKTDGET | SNRAFDI |
| (SEQ ID NO: 21) | (SEQ ID NO: 22) | (SEQ ID NO: 23) |
| KSSQSVLYAGNNRNYLA | QASTRAS | QQYYTPPLA |
| (SEQ ID NO: 24) | (SEQ ID NO: 25) | (SEQ ID NO: 26) |
| GLTFERAW | IKRKTDGETT | AGSNRAFDI |
| (SEQ ID NO: 27) | (SEQ ID NO: 28) | (SEQ ID NO: 29) |
| QSVLYAGNNRNY | QA | QQYYTPPLA |
| (SEQ ID NO: 30) | (SEQ ID NO: 31) | (SEQ ID NO: 32) |
| GLTFERAWMN | RIKRKTDGETTD | SNRAFDI |
| (SEQ ID NO: 33) | (SEQ ID NO: 34) | (SEQ ID NO: 35) |
| KSSQSVLYAGNNRNYLA | QASTRAS | QQYYTPPLA |
| (SEQ ID NO: 36) | (SEQ ID NO: 37) | (SEQ ID NO: 38) |
| ERAWMN | WVGRIKRKTDGETTD | AGSNRAFD |
| (SEQ ID NO: 39) | (SEQ ID NO: 40) | (SEQ ID NO: 41) |
| LYAGNNRNYLAWY | LLINQASTRA | QQYYTPPL |
| (SEQ ID NO: 42) | (SEQ ID NO: 43) | (SEQ ID NO: 44) |

In some embodiments, the N-terminal amino acid of the V_{H} domain of the anti-CD47 antibody (or immunologically active fragment thereof) corresponds to position H1 according to the Kabat numbering system, and the C-terminal amino acid of the V_{H} domain of the anti-CD47 antibody (or immunologically active fragment thereof) corresponds to position H113 according to the Kabat numbering system. In some embodiments, the N-terminal amino acid of the V_{H} domain of the anti-CD47 antibody (or immunologically active fragment thereof) corresponds to position H1 according to the Chothia numbering system, and the C-terminal amino acid of the V_{H} domain of the anti-CD47 antibody (or immunologically active fragment thereof) corresponds to position H113 according to the Chothia numbering system. In some embodiments, the N-terminal amino acid of the V_{H} domain of the anti-CD47 antibody (or immunologically active fragment thereof) corresponds to position H1 according to the IMGT numbering system, and the C-terminal amino acid of the V_{H} domain of the anti-CD47 antibody (or immunologically active fragment thereof) corresponds to position H128 according to the IMGT numbering system. In some embodiments, the N-terminal amino acid of the V_{H} domain of the anti-CD47 antibody (or immunologically active fragment thereof) corresponds to amino acid 1 of SEQ ID NO: 1, and the C-terminal amino acid of the V_{H} domain the anti-CD47 antibody (or immunologically active fragment thereof) corresponds to amino acid 118 of SEQ ID NO: 1.

In some embodiments, the anti-CD47 antibody (or immunologically active fragment thereof) comprises a heavy chain variable domain (VH) comprising an amino acid sequence that has at least about 95%, 96%, 97%, 98%, 99%, or 100% sequence identity to an amino acid sequence set forth in SEQ ID NO: 1, provided that the N-terminal amino acid of the VH domain is an E and the C-terminal amino acid of the VH domain is an S, and optionally a light chain variable domain (VL) comprising an amino acid sequence that has at least about 95%, 96%, 97%, 98%, 99%, or 100% sequence identity to an amino acid sequence set forth in SEQ ID NO: 2. The amino acid sequences of SEQ ID NOs: 1 and 2 are provided below:

(SEQ ID NO: 1)
EVQLVESGGG LVKPGGSLRL SCAASGLTFE RAWMNWVRQA
PGKGLEWVGR IKRKTDGETT DYAAPVKGRF SISRDDSKNT
LYLQMNSLKT EDTAVYYCAG SNRAFDIWGQ GTMVTVSS
(SEQ ID NO: 2)
DIVMTQSPDS LAVSLGERAT INCKSSQSVL YAGNNRNYLA
WYQQKPGQPP KLLINQASTR ASGVPDRESG SGSGTEFTLI
ISSLQAEDVA IYYCQQYYTP PLAFGGGTKL EIK

In some embodiments, the anti-CD47 antibody (or immunologically active fragment thereof) comprises 3 CDRs of a VH domain comprising SEQ ID NO: 1, provided that the N-terminal amino acid of the VH domain is an E and the C-terminal amino acid of the VH domain is an S. Additionally or alternatively, in some embodiments, the anti-CD47 antibody (or immunologically active fragment thereof) comprises 3 CDRs of a VL domain comprising SEQ ID NO: 2. In some embodiments, the 3 CDRs of the VH domain are CDRs according to Kabat, Chothia, AbM or Contact numbering scheme. Additionally or alternatively, in some embodiments, the 3 CDRs of the VL domain are CDRs according to Kabat, Chothia, AbM or Contact numbering scheme. In some embodiments, the VH domain of the anti-CD47 antibody comprises an amino acid sequence that is at least about 95%, 96%, 97%, 98%, 99%, or 100% identity to the amino acid sequence set forth in SEQ ID NO:1, provided that the N-terminal amino acid of the VH domain is an E and the C-terminal amino acid of the VH domain is an S. Additionally or alternatively, in some embodiments, the VL domain of the anti-CD47 antibody comprises an amino acid sequence that is at least about 95%, 96%, 97%, 98%, 99%, or 100% identity to the amino acid sequence set forth in SEQ ID NO:2. In some embodiments, the anti-CD47 antibody comprises a VH comprising SEQ ID NO: 1 and a VL comprising SEQ ID NO: 2. In some embodiments, the anti-CD47 antibody is a full length antibody that comprises a heavy chain comprising the amino acid SEQ ID NO: 3 and a light chain comprising the amino acid sequence of SEQ ID NO: 4. In some embodiments, the anti-CD47 antibody is a full length antibody that comprises a heavy chain comprising the amino acid SEQ ID NO: 55 and a light chain comprising the amino acid sequence of SEQ ID NO: 4.

(SEQ ID NO: 3)
EVQLVESGGG LVKPGGSLRL SCAASGLTFE RAWMNWVRQA PGKGLEWVGR IKRKTDGETT
DYAAPVKGRF SISRDDSKNT LYLQMNSLKT EDTAVYYCAG SNRAFDIWGQ GTMVTVSSAS
TKGPSVFPLA PCSRSTSEST AALGCLVKDY FPEPVTVSWN SGALTSGVHT FPAVLQSSGL
YSLSSVVTVP SSSLGTKTYT CNVDHKPSNT KVDKRVESKY GPPCPPCPAP EFLGGPSVEL
FPPKPKDTLM ISRTPEVTCV VVDVSQEDPE VQFNWYVDGV EVHNAKTKPR EEQFNSTYRV
VSVLTVLHQD WINGKEYKCK VSNKGLPSSI EKTISKAKGQ PREPQVYTLP PSQEEMTKNQ
VSLTCLVKGF YPSDIAVEWE SNGQPENNYK TTPPVLDSDG SFFLYSRLTV DKSRWQEGNV
FSCSVMHEAL HNHYTQKSLS LSLGK
(SEQ ID NO: 55)
EVQLVESGGG LVKPGGSLRL SCAASGLTFE RAWMNWVRQA PGKGLEWVGR IKRKTDGETT
DYAAPVKGRF SISRDDSKNT LYLQMNSLKT EDTAVYYCAG SNRAFDIWGQ GTMVTVSSAS
TKGPSVFPLA PCSRSTSEST AALGCLVKDY FPEPVTVSWN SGALTSGVHT FPAVLQSSGL
YSLSSVVTVP SSSLGTKTYT CNVDHKPSNT KVDKRVESKY GPPCPPCPAP EFLGGPSVEL
FPPKPKDTLM ISRTPEVTCV VVDVSQEDPE VQFNWYVDGV EVHNAKTKPR EEQFNSTYRV
VSVLTVLHQD WINGKEYKCK VSNKGLPSSI EKTISKAKGQ PREPQVYTLP PSQEEMTKNQ
VSLTCLVKGF YPSDIAVEWE SNGQPENNYK TTPPVLDSDG SFFLYSRLTV DKSRWQEGNV
FSCSVMHEAL HNHYTQKSLS LSLG
(SEQ ID NO: 4)
DIVMTQSPDS LAVSLGERAT INCKSSQSVL YAGNNRNYLA WYQQKPGQPP KLLINQASTR
ASGVPDRESG SGSGTEFTLI ISSLQAEDVA IYYCQQYYTP PLAFGGGTKL EIKRTVAAPS
VFIFPPSDEQ LKSGTASVVC LLNNFYPREA KVQWKVDNAL QSGNSQESVT EQDSKDSTYS
LSSTLTLSKA DYEKHKVYAC EVTHQGLSSP VTKSENRGEC

In some embodiments, the anti-CD47 antibody (or immunologically active fragment thereof) binds to (e.g., cross-reacts with) CD47 from at least two different species. In some embodiments, for example, the anti-CD47 antibody (or antibody variant) binds to a hCD47 protein (or the extracellular domain thereof) and a CD47 (or the extracellular domain thereof) from a non-human primate (such as a cynomolgus or rhesus monkey). In some embodiments, the anti-CD47 antibody may be completely specific for human CD47 and may not exhibit species or other types of non-human cross-reactivity.

The anti-CD47 antibody that binds specifically to hCD47 can be of any of the various types of antibodies as defined above, but is, in certain embodiments, a human, humanized, or chimeric antibody. In some embodiments, the anti-CD47 antibody is a human antibody. In some embodiments, the anti-CD47 is a humanized antibody or comprises a human antibody constant domain (e.g., a human Fc domain, such as a human IgG Fc domain, e.g., a human IgG1, a human IgG2, a human IgG3, or a human IgG4 Fc domain). In some embodiments, an antibody of the present disclosure is a chimeric antibody. See, e.g., U.S. Pat. No. 4,816,567 and Morrison et al., Proc. Natl. Acad. Sci. USA, 81:6851-6855 (1984). In some embodiments, a chimeric antibody comprises a non-human variable region (e.g., a variable region derived from a chicken, mouse, rat, hamster, rabbit, or non-human primate, such as a monkey) and a human constant region. In some embodiments, a chimeric antibody is a “class switched” antibody in which the class or subclass has been changed from that of the parent antibody. Chimeric antibodies include antigen-binding fragments thereof.

In some embodiments, a chimeric antibody is a humanized antibody. A non-human antibody can be humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody (e.g., a chicken antibody), and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR or CDR residues are derived), e.g., to restore or improve antibody specificity or affinity. Humanized antibodies and methods of making them are reviewed, e.g., in Almagro and Fransson, Front. Biosci. 13:1619-1633 (2008).

Human framework regions useful for humanization include but are not limited to: framework regions selected using the “best-fit” method; framework regions derived from the consensus sequence of human antibodies of a particular subgroup of light or heavy chain variable regions; human somatically mutated framework regions or human germline framework regions; and framework regions derived from screening FR libraries. See, e.g., Sims et al. J. Immunol. 151:2296 (1993); Carter et al. Proc. Natl. Acad. Sci. USA, 89:4285 (1992); Presta et al. J. Immunol., 151:2623 (1993); Almagro and Fransson, Front. Biosci. 13:1619-1633 (2008); and Baca et al., J. Biol. Chem. 272:10678-10684 (1997).

In some embodiments, an anti-CD47 antibody of the present disclosure is a human antibody. Human antibodies can be produced using various techniques known in the art. In some embodiments, the human antibody is produced by a non-human animal, such as the genetically engineered chickens (see, e.g., U.S. Pat. Nos. 8,592,644; and 9,380,769) and/or mice described herein. Human antibodies are described generally in Lonberg, Curr. Opin. Immunol. 20:450-459 (2008).

In some embodiments, an anti-CD47 antibody of the present disclosure is an antibody fragment, including without limitation a Fab, F(ab′)₂, Fab′-SH, Fv, or scFv fragment, or a single domain, single heavy chain, or single light chain antibody. Antibody fragments can be generated, e.g., by enzymatic digestion or by recombinant techniques. In some embodiments, Proteolytic digestion of an intact antibody is used to generate an antibody fragment, e.g., as described in Morimoto et al., Journal of Biochemical and Biophysical Methods 24:107-117 (1992) and Brennan et al., Science, 229:81 (1985). In some embodiments, an antibody fragment is produced by a recombinant host cell. For example, Fab, Fv and ScFv antibody fragments are expressed by and secreted from E. coli. Antibody fragments can alternatively be isolated from an antibody phage library.

Fab′-SH fragments can be directly recovered from E. coli and chemically coupled to form F(ab′)₂ fragments. See Carter et al., Bio/Technology 10:163-167 (1992). F(ab′)₂ fragments can also be isolated directly from a recombinant host cell culture. Fab and F(ab′)₂ fragment with increased in vivo half-life comprising salvage receptor binding epitope residues are described in U.S. Pat. No. 5,869,046.

In some embodiments, an antibody is a single chain Fv fragment (scFv). See WO 93/16185 and U.S. Pat. Nos. 5,571,894 and 5,587,458. scFv fusion proteins can be constructed to produce a fusion of an effector protein at either the amino or the carboxy terminus of an scFv. The antibody fragment may also be a “linear antibody”, e.g., as described in U.S. Pat. No. 5,641,870, for example. Such linear antibodies may be monospecific or bispecific.

In some embodiments, the anti-CD47 antibody (or immunologically active fragment thereof) specifically recognizes (such as binds) to hCD47 expressed on the surface of a cell. In some embodiments, the anti-CD47 antibody specifically recognizes hCD47 expressed on the surface of a cancer cell. In some embodiments, the anti-CD47 antibody specifically recognizes hCD47 expressed on the cell surface of a cancer cell line including, but not limited to, e.g. SK-OV-3, Toledo, K562, HCC827, Jurkat, U937, TF-1, Raji, SU-DHL-4, MDA-MB-231, A375, and SK-MES-1 cell lines. In some embodiments, the anti-CD47 antibody specifically recognizes hCD47 expressed on the cell surface of cancer cells in a subject (e.g., lung cancer cells, an ovarian cancer cells, colorectal cancer cells, pancreatic cancer cells, sarcoma cancer cells, head and neck cancer cells, gastric cancer cells, renal cancer cells, skin cancer cells, and non-Hodgkin lymphoma cells). In some embodiments, the binding of an anti-CD47 antibody (or immunologically active fragment thereof) described herein to hCD47 (e.g., hCD47 expressed on the surface of a cell) prevents the interaction of hCD47 with signal regulatory protein alpha (SIRPα), such as human SIRPα (“hSIRPα”). In some embodiments, the binding of an anti-CD47 antibody (or immunologically active fragment thereof) described herein to hCD47 expressed on the surface of a cancer cell promotes macrophage mediated phagocytosis of the cancer cell. In some embodiments, the anti-CD47 antibody or immunologically active fragment thereof does not bind to hCD47 expressed on the surface of a blood cell. In some embodiments, the administration of an anti-CD47 antibody (or immunologically active fragment thereof) described herein to a subject (e.g., a human or non-human primate) does not induce or cause significant hematological toxicity (e.g., anemia, cytopenia, or hemagglutination) in the subject or significant depletion of the subject's red blood cells. In some embodiments, the administration of an anti-CD47 antibody (or immunologically active fragment thereof) described herein to a subject (e.g., a human or non-human primate) does not induce or cause hematological toxicity (e.g., anemia, cytopenia, or hemagglutination) in the subject or depletion of the subject's red blood cells.

Nucleic Acids Encoding Anti-CD47 Antibodies

Nucleic acid molecules encoding the anti-CD47 antibodies (or immunologically active fragments thereof) described herein are also contemplated. In some embodiments, provided is a nucleic acid (or a set of nucleic acids) encoding an anti-CD47 antibody, including any of the anti-CD47 antibodies described herein. In some embodiments, the nucleic acid sequence(s) (such as a DNA sequence(s)) encoding an anti-CD47 antibody (or immunologically active fragment thereof) have been optimized (such as further optimized) to maximize translation and stability of an RNA transcribed from the nucleic acid(s), e.g., for the purpose of increasing production yield of the anti-CD47 antibody during the manufacturing process. Exemplary optimizations include, but are not limited to, e.g., removing repeated sequences, removing killer motifs and splice sites, reducing GC content (guanine-cytosine content), removing/replacing sequences that may form mRNA secondary structures or unstable motifs, and/or optimizing of codon usage in a given host cell (e.g., a CHO cell, such as a CHO-K1 cell). Codon optimization is a process used to improve gene expression and increase the translational efficiency of a nucleic acid of interest by accommodating codon bias and tRNA frequency of the host organism. In some embodiments, the nucleic acid(s) encoding an anti-CD47 antibody described herein has been codon-optimized, e.g., codon-optimized for expression in a CHO cell (such as a CHO-K1 cell).

In some embodiments, a nucleic acid encoding the V_{H} domain of an anti-CD47 antibody provided herein comprises a polynucleotide that has at least about 95%, 96%, 97%, 98%, 99%, or 100% sequence identity to SEQ ID NO: 45, provided that the amino acid sequence of the VH domain encoded by the nucleic acid comprises an N-terminal amino E and a C-terminal S. In some embodiments, a nucleic acid encoding the V_{L} domain of an anti-CD47 antibody provided herein comprises a polynucleotide that has at least about 95%, 96%, 97%, 98%, 99%, or 100% sequence identity to SEQ ID NO: 46. SEQ ID NOs: 45 and 46 are provided below:

(SEQ ID NO: 45)
GAGGTGCAGCTGGTGGAGAGCGGAGGCGGACTCGTGAAGCCTGGAGGAA
GCCTGAGGCTGTCCTGTGCCGCTTCCGGCCTCACCTTCGAGCGGGCTTG
GATGAACTGGGTGAGGCAGGCCCCTGGAAAGGGCCTGGAATGGGTGGGC
CGGATCAAGAGGAAAACAGATGGCGAGACCACCGATTACGCCGCTCCCG
TGAAGGGCCGGTTTAGCATCTCCAGGGACGACTCCAAGAACACCCTGTA
TCTGCAGATGAACAGCCTGAAGACCGAGGACACCGCTGTGTACTACTGC
GCTGGCAGCAACAGGGCCTTTGATATCTGGGGCCAGGGCACCATGGTGA
CAGTGTCCTCC
(SEQ ID NO: 46)
GACATCGTGATGACCCAGTCCCCTGATTCCCTGGCCGTGAGCCTGGGCG
AAAGGGCTACCATCAACTGCAAGTCCTCCCAGAGCGTGCTGTACGCCGG
CAACAACCGGAACTATCTGGCTTGGTACCAGCAGAAGCCCGGCCAGCCT
CCCAAGCTGCTGATCAACCAGGCTAGCACCAGGGCTTCCGGCGTGCCTG
ATAGGTTCAGCGGCTCCGGCTCCGGCACCGAGTTTACCCTGATCATCTC
CTCCCTGCAGGCCGAGGATGTGGCCATCTACTACTGCCAGCAGTACTAC
ACCCCTCCTCTGGCCTTTGGCGGCGGCACCAAGCTGGAGATCAAG

In some embodiments, the nucleic acid (or set of nucleic acids) encoding an anti-CD47 antibody described herein may further comprises a nucleic acid sequence encoding a peptide tag (such as protein purification tag, e.g., His-tag, HA tag). In some embodiments, the nucleic acid (or set of nucleic acids) encoding an anti-CD47 antibody (or an immunologically active fragment thereof) comprises a leader sequence. In some embodiments, provided are nucleic acids comprising nucleotide sequences that hybridize to the nucleic acid sequences encoding an anti-CD47 antibody described herein under at least moderately stringent hybridization conditions.

Also provided are vectors in which a nucleic acid described herein is inserted.

In brief summary, the expression of an anti-CD47 antibody (or antigen binding fragment thereof) by a natural or synthetic nucleic acid encoding the anti-CD47 antibody (or antigen binding fragment thereof) can be achieved by inserting the nucleic acid into an appropriate expression vector, such that the nucleic acid is operably linked to 5′ and 3′ regulatory elements, including for example a promoter (e.g., a constitutive, regulatable, tissue-specific promoter) and a 3′ untranslated region (UTR). The vectors can be suitable for replication and integration in eukaryotic host cells. Typical cloning and expression vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers (see, e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity.

Additional promoter elements, e.g., enhancers, regulate the frequency of transcriptional initiation. Typically, these are located in the region 30-110 base pairs (bp) upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1a (EF-1a). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the expression of the nucleic acid(s) encoding the anti-CD47 antibody (or immunologically active fragment thereof) is inducible. In some embodiments, the nucleic acid(s) encoding the anti-CD47 antibody (or immunologically active fragment thereof) is operably linked to an inducible promoter, including any inducible promoter known in the art. In some embodiments, the nucleic acid(s) encoding the an anti-CD47 antibody described herein has been engineered to encode an epitope tag, e.g., to facilitate purification or detection of the antibody. Exemplary epitope tags include, but are not limited to, e.g., 6× His (also known as His-tag or hexahistidine tag), FLAG, HA, Myc, V5, GFP (green fluorescent protein, e.g., enhanced green fluorescent protein or EGFP), GST (glutathione-S-transferase), β-GAL (β-galactosidase), Luciferase, MBP (Maltose Binding Protein), RFP (Red Fluorescence Protein), and VSV-G (Vesicular Stomatitis Virus Glycoprotein).

Methods of Antibody Production

An anti-CD47 antibody (or immunologically active fragment thereof) of the present disclosure may be produced by any means known in the art. Exemplary techniques for antibody production are described below; however these exemplary techniques are provided for illustrative purposes only and are not intended to be limiting. In addition, exemplary antibody properties contemplated for use with the antibodies described herein are further described.

To prepare an antigen, the antigen may be purified or otherwise obtained from a natural source, or it may be expressed using recombinant techniques. In some embodiments, the antigen may be used as a soluble protein. In some embodiments, the antigen may be conjugate to another polypeptide or other moiety, e.g., to increase its immunogenicity. For example, an antigen described herein may be coupled with an Fc region. In some embodiments, a cell expressing the antigen on its cell surface may be used as the antigen.

Polyclonal antibodies can be raised in an animal by multiple subcutaneous (sc) or intraperitoneal (ip) injections of the antigen and an adjuvant. For example, descriptions of chicken immunization are described herein. In some embodiments, the antigen is conjugated with an immunogenic protein, e.g., keyhole limpet hemocyanin, serum albumin, bovine thyroglobulin, or soybean trypsin inhibitor using a bifunctional or derivatizing agent. Exemplary methods for immunization of chickens are provided herein. Relevant methods suitable for a variety of other organisms, such as mammals, are well known in the art.

As described supra, monoclonal antibodies may be produced by a variety of methods. In some embodiments, a monoclonal antibody of the present disclosure is made using the hybridoma method first described by Kohler et al., Nature, 256:495 (1975), and further described in Hongo et al., Hybridoma, 14 (3): 253-260 (1995); Harlow et al., Antibodies: A Laboratory Manual, (Cold Spring Harbor Laboratory Press, 2nd ed. 1988); and Hammerling et al., in: Monoclonal Antibodies and T-Cell Hybridomas 563-681 (Elsevier, N.Y., 1981). Human hybridoma technology (Trioma technology) is described in Vollmers and Brandlein, Histology and Histopathology, 20(3):927-937 (2005) and Vollmers and Brandlein, Methods and Findings in Experimental and Clinical Pharmacology, 27(3):185-91 (2005). A culture medium in which hybridoma cells are grown may be screened for the presence of an antibody of interest, e.g., by in vitro binding assay, immunoprecipitation, ELISA, RIA, etc.; and the binding affinity may be determined, e.g., by Scatchard analysis. A hybridoma that produces an antibody with desired binding properties can be subcloned and grown using known culture techniques, grown in vivo as ascites tumors in an animal, and the like.

In some embodiments, a monoclonal antibody is made using a library method, such as a phage display library. See, e.g., Hoogenboom et al. in Methods in Molecular Biology 178:1-37 (O'Brien et al., ed., Human Press, Totowa, N J, 2001). In some embodiments, repertoires of VH and VL genes are cloned by polymerase chain reaction (PCR) and recombined randomly in phage libraries, which are then screened for antigen-binding phage, e.g., as described in Winter et al., Ann. Rev. Immunol., 12: 433-455 (1994). Phage typically display antibody fragments, either as single-chain Fv (scFv) fragments or as Fab fragments. Alternatively, the naive repertoire can be cloned (e.g., from human) to provide a single source of antibodies to a wide range of non-self and also self-antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992).

Antibodies can be produced using recombinant methods. For recombinant production of an anti-antigen antibody, nucleic acid encoding the antibody is isolated and inserted into a replicable vector for further cloning (amplification of the DNA) or for expression. DNA encoding the antibody may be readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

An antibody of the present disclosure can be produced recombinantly as a fusion polypeptide with a heterologous polypeptide, e.g., a signal sequence or other polypeptide having a specific cleavage site at the N-terminus of the mature protein or polypeptide. The heterologous signal sequence selected can be one that is recognized and processed (e.g., cleaved by a signal peptidase) by the host cell. For prokaryotic host cells that do not recognize and process a native antibody signal sequence, the signal sequence is substituted by a prokaryotic signal sequence selected, for example, from alkaline phosphatase, penicillinase, 1pp, or heat-stable enterotoxin II leaders. For yeast secretion the native signal sequence may be substituted by, e.g., the yeast invertase leader, a factor leader (including Saccharomyces and Kluyveromyces α-factor leaders), or acid phosphatase leader, the C. albicans glucoamylase leader, etc. In mammalian cell expression, mammalian signal sequences as well as viral secretory leaders, for example, the herpes simplex gD signal, are available.

Both expression and cloning vectors contain a nucleic acid sequence that enables the vector to replicate in one or more selected host cells, e.g., to allow the vector to replicate independently of the host chromosomal DNA. This sequence can include origins of replication or autonomously replicating sequences. Such sequences are well known for a variety of bacteria, yeast, and viruses. Generally, the origin of replication component is not needed for mammalian expression vectors (the SV40 origin may be used because it contains the early promoter).

Expression and cloning vectors can contain a selection gene or selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media. Examples of dominant selection use the drugs neomycin, mycophenolic acid and hygromycin. Another example of suitable selectable markers for mammalian cells are those that enable the identification of cells competent to take up antibody-encoding nucleic acid, such as DHFR, glutamine synthetase (GS), thymidine kinase, metallothionein-I and -II, preferably primate metallothionein genes, adenosine deaminase, ornithine decarboxylase, and the like. For example, a Chinese hamster ovary (CHO) cell line deficient in endogenous DHFR activity transformed with the DHFR gene is identified by culturing the transformants in a culture medium containing methotrexate (Mtx), a competitive antagonist of DHFR.

Alternatively, host cells (particularly wild-type hosts that contain endogenous DHFR) transformed or co-transformed with DNA sequences encoding an antibody of interest, wild-type DHFR gene, and another selectable marker such as aminoglycoside 3′-phosphotransferase (APH) can be selected by cell growth in medium containing a selection agent for the selectable marker such as an aminoglycosidic antibiotic, e.g., kanamycin, neomycin, or G418.

Expression and cloning vectors generally contain a promoter that is recognized by the host organism and is operably linked to nucleic acid encoding an antibody. Promoters suitable for use with prokaryotic hosts include thephoA promoter, β-lactamase and lactose promoter systems, alkaline phosphatase promoter, a tryptophan (trp) promoter system, and hybrid promoters such as the tac promoter. However, other known bacterial promoters are suitable. Promoter sequences are known for eukaryotes. Yeast promoters are well known in the art and can include inducible promoters/enhancers regulated by growth conditions. Virtually all eukaryotic genes have an AT-rich region located approximately 25 to 30 bases upstream from the site where transcription is initiated. Examples include without limitation the promoters for 3-phosphoglycerate kinase or other glycolytic enzymes, such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase. Antibody transcription from vectors in mammalian host cells can be controlled, for example, by promoters obtained from the genomes of viruses. The early and late promoters of the SV40 virus are conveniently obtained as an SV40 restriction fragment that also contains the SV40 viral origin of replication. The immediate early promoter of the human cytomegalovirus is conveniently obtained as a HindIII E restriction fragment. Alternatively, the Rous Sarcoma Virus long terminal repeat can be used as the promoter.

Transcription of a DNA encoding an antibody of this invention by higher eukaryotes is often increased by inserting an enhancer sequence into the vector. Many enhancer sequences are now known from mammalian genes (globin, elastase, albumin, α-fetoprotein, and insulin). Typically, however, one will use an enhancer from a eukaryotic cell virus.

Expression vectors used in eukaryotic host cells (yeast, fungi, insect, plant, animal, human, or nucleated cells from other multicellular organisms) will also contain sequences necessary for the termination of transcription and for stabilizing the mRNA.

Suitable host cells for cloning or expressing the DNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, etc. In addition to prokaryotes, eukaryotic microbes such as filamentous fungi or yeast are suitable cloning or expression hosts for antibody-encoding vectors. Saccharomyces cerevisiae, or common baker's yeast, is the most commonly used among lower eukaryotic host microorganisms. Certain fungi and yeast strains may be selected in which glycosylation pathways have been “humanized,” resulting in the production of an antibody with a partially or fully human glycosylation pattern. See, e.g., Li et al., Nat. Biotech. 24:210-215 (2006).

Plant cell cultures of cotton, corn, potato, soybean, petunia, tomato, duckweed (Leninaceae), alfalfa (M. truncatula), and tobacco can also be utilized as hosts.

Suitable host cells for the expression of glycosylated antibody are also derived from multicellular organisms (invertebrates and vertebrates). Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified.

Vertebrate cells may be used as hosts, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as NS0 and Sp2/0. For a review of certain mammalian host cell lines suitable for antibody production, see, e.g., Yazaki and Wu, Methods in Molecular Biology, Vol. 248 (B. K C. Lo, ed., Humana Press, Totowa, N.J., 2003), pp. 255-268. In some embodiments, the host cell is a CHO-K1 cell. The CHO-K1 cell line is a subclone of CHO cell line (see, e.g., www(dot)phe-culturecollections(dot)org(dot)uk/media/128263/chok1-cell-line-profile(dot)pdf and web(dot)expasy(dot)org/cellosaurus/CVCL_0214.

The host cells of the present disclosure (e.g., a CHO-K1 cell) may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to one of skill in the art.

When using recombinant techniques, the antibody can be produced intracellularly, in the periplasmic space, or directly secreted into the medium. If the antibody is produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli.

The antibody composition prepared from the cells can be purified using, for example, hydroxylapatite chromatography, hydrophobic interaction chromatography, gel electrophoresis, dialysis, and affinity chromatography, with affinity chromatography being among one of the typically preferred purification steps. In some embodiments, an anti-CD47 antibody described herein comprises an epitope tag (e.g., a tag attached to the antibody via a cleavable linker) to facilitate purification. Exemplary epitope tags include, but are not limited to, e.g., e.g., 6× His (also known as His-tag or hexahistidine tag), FLAG, HA, Myc, V5, GFP (green fluorescent protein, e.g., enhanced green fluorescent protein or EGFP), GST (glutathione-S-transferase), β-GAL ((3-galactosidase), Luciferase, MBP (Maltose Binding Protein), RFP (Red Fluorescence Protein), and VSV-G (Vesicular Stomatitis Virus Glycoprotein.

Thus, in some embodiments, provided is a method of making an anti-CD47 antibody (or an immunologically active fragment thereof) that comprises (a) a heavy chain variable (V_{H}) domain that comprises (1) a glutamic acid residue (E) at its N-terminus; (2) a CDR-H1 comprising RAWMN (SEQ ID NO: 5); (3) a CDR-H2 comprising RIKRKTDGETTDYAAPVKG (SEQ ID NO: 6); (4) a CDR-H3 comprising SNRAFDI (SEQ ID NO: 7); and (5) a serine (S) at its C-terminus; and (b) a light chain variable (V_{L}) domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10), the method comprising: a) culturing a host cell (such as a CHO cell) that comprises a nucleic acid that encodes the anti-CD47 antibody (or immunologically active fragment thereof) under conditions effective to cause expression of the antibody (or fragment); and b) recovering the anti-CD47 antibody (or fragment thereof) expressed by the host cell. In some embodiments, the method further comprises the step of c) purifying the antibody. In some embodiments, purifying the anti-CD47 antibody comprises at least one chromatography step, such as a Protein A or Protein L chromatography step. In some embodiments the host cell is a mammalian cell. In some embodiments, the host cell is a CHO cell, e.g., a CHO-K1 cell. In some embodiments the host cell comprises one or more vectors that encode the anti-CD47 antibody. In some embodiments, the host cell has been transfected (e.g., transiently transfected or stably transfected) with nucleic acid(s) that encode the anti-CD47 antibody. In some embodiments, the method of making an anti-CD47 antibody is a manufacturing-scale production process (such as a fermentation process). In some embodiments, a “manufacturing-scale” production process (e.g., fermentation process) of making an anti-CD47 antibody entails culturing and growing the host cell in a culture volume ranging between about 400L to about 80,000 L (such as between about 400 L to about 25,000 L, e.g., about any one of 4,000 L, 6,000 L, 8,000 L, 10,000 L, 12,000 L, 14,000 L, or 16,000 L).

Glycosylation Variants

In some embodiments, an anti-CD47 antibody (or immunologically active fragment thereof) provided herein is altered to increase or decrease the extent to which the anti-CD47 antibody (or immunologically active fragment thereof) is glycosylated. Addition or deletion of glycosylation sites to an anti-CD47 antibody (or immunologically active fragment thereof) may be conveniently accomplished by altering the amino acid sequence of the anti-CD47 antibody (or immunologically active fragment thereof) or polypeptide portion thereof such that one or more glycosylation sites is created or removed.

Where the anti-CD47 antibody (or immunologically active fragment thereof) comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al., TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an anti-CD47 antibody (or immunologically active fragment thereof) herein may be made in order to create anti-CD47 antibody variants (or immunologically active fragments thereof comprising an Fc region) with certain improved properties.

The N-glycans attached to the CH2 domain of Fc is heterogeneous. Antibodies or Fc fusion proteins generated in CHO cells are fucosylated by fucosyltransferase activity. See Shoji-Hosaka et al., J. Biochem. 2006, 140:777-83. Normally, a small percentage of naturally occurring afucosylated IgGs may be detected in human serum. N-glycosylation of the Fc is important for binding to FcγR; and afucosylation of the N-glycan increases Fc's binding capacity to FcγRIIIa. Increased FcγRIIIa binding can enhance ADCC, which can be advantageous in certain antibody therapeutic applications in which cytotoxicity is desirable.

In some embodiments, an enhanced effector function can be detrimental when Fc-mediated cytotoxicity is undesirable. In some embodiments, the Fc fragment or CH2 domain is not glycosylated. In some embodiments, the N-glycosylation site in the CH2 domain is mutated to prevent from glycosylation.

In some embodiments, anti-CD47 antibody variants (or immunologically active fragments thereof) are provided comprising an Fc region wherein a carbohydrate structure attached to the Fc region has reduced fucose or lacks fucose, which may improve ADCC function.

Immunoconjugates and Covalent Modifications

The invention also pertains to immunoconjugates comprising an antibody conjugated to second moiety. In some embodiments, the second moiety is a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, an ¹⁸⁶Re. Exemplary chemotherapeutic agents useful in the generation of such immunoconjugates are described elsewhere herein.

In certain embodiments, a humanized anti-CD47 antibody provided herein (or an antigen-binding fragment thereof) is conjugated to maytansine, a maytansinoid, or calicheamicin. In certain embodiments, a humanized anti-CD47 antibody provided herein (or an antigen-binding fragment thereof) is conjugated to the maytansinoid DM1.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bisdiazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See, WO94/11026.

In another embodiment, the antibody can be conjugated to a “receptor” (such as streptavidin) for utilization in tumor pre-targeting wherein the antibody-receptor conjugate is administered to the patient, followed by removal of unbound conjugate from the circulation using a clearing agent and then administration of a “ligand” (e.g., avidin) that is conjugated to a cytotoxic agent (e.g., a radionucleotide).

Also provided are heteroconjugate antibodies comprising a humanized anti-CD47 antibody described herein covalently joined to at least one other antibody. Heteroconjugate antibodies have, for example, been proposed to target immune-system cells to unwanted cells (U.S. Pat. No. 4,676,980), and for treatment of HIV infection. Heteroconjugate antibodies comprising a humanized anti-CD47 antibody described herein can be prepared in vitro using known methods in synthetic protein chemistry, including those involving crosslinking agents. For example, immunotoxins can be constructed using a disulfide-exchange reaction or by forming a thioether bond. Examples of suitable reagents for this purpose include iminothiolate and methyl-4-mercaptobutyrimidate and those disclosed, for example, in U.S. Pat. No. 4,676,980.

Also provided is a humanized anti-CD47 antibody comprising at least one covalent modification. One type of covalent modification includes reacting targeted amino acid residues of a humanized anti-CD47 with an organic derivatizing agent that is capable of reacting with selected side chains or the N- or C-terminal residues of the antibody. Commonly used crosslinking agents include, but are not limited to, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidyl-propionate), bifunctional maleimides such as bis-N-maleimido-1,8-octane and agents such as methyl-3-[(p-azidophenyl)-dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification comprises linking a humanized anti-CD47 antibody provided herein (or an antigen-binding fragment thereof) to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

Cysteine Engineered Variants

In some embodiments, it may be desirable to create cysteine engineered anti-CD47 antibodies in which one or more amino acid residues are substituted with cysteine residues. In some embodiments, the substituted residues occur at accessible sites of the anti-CD47 antibody. By substituting those residues with cysteine, reactive thiol groups are thereby positioned at accessible sites of the anti-CD47 antibody and may be used to conjugate the anti-CD47 antibody to other moieties, such as drug moieties or linker-drug moieties, to create an anti-CD47 immunoconjugate, as described further herein. Cysteine engineered anti-CD47 antibodies may be generated as described, e.g., in U.S. Pat. No. 7,521,541.

Effector Function Engineering

It may be desirable to modify an anti-CD47 antibody provided herein with respect to effector function, so as to enhance, e.g., the effectiveness of the antibody in treating cancer. For example, cysteine residue(s) can be introduced into the Fc region, thereby allowing inter-chain disulfide bond formation in this region. The homodimeric antibody thus generated can have improved internalization capability and/or increased complement-mediated cell killing and antibody-dependent cellular cytotoxicity (ADCC). See, Caron et al., J Exp. Med., 176: 1191-1195 (1992) and Shapes, J Immunol., 148: 2918-2922 (1992). Homodimeric antibodies with enhanced anti-tumor activity can also be prepared using heterobifunctional cross-linkers as described in Wolff et al., Cancer Research, 53: 2560-2565 (1993). Alternatively, an antibody can be engineered to comprise usual Fc regions and can thereby have enhanced complement lysis and ADCC capabilities. See, Stevenson et al., Anti-Cancer Drug Design3: 219-230 (1989).

Mutations or alterations in the Fc region sequences can be made to improve FcR binding (e.g., binding to FcγR, FcRn). In some embodiments, an anti-CD47 antibody provided herein comprises at least one altered effector function, e.g., altered ADCC, CDC, and/or FcRn binding compared to a native IgG or a parent antibody. In some embodiments, the effector function of the antibody comprising the mutation or alteration is increased relative to the parent antibody. In some embodiments, the effector function of the antibody comprising the mutation or alteration is decreased relative to the parent antibody. Examples of several useful specific mutations are described in, e.g., Shields, R L et al. (2001) JBC 276(6)6591-6604; Presta, L. G., (2002) Biochemical Society Transactions 30(4):487-490; and WO 00/42072.

In some embodiments, an anti-CD47 antibody provided herein comprises an Fc receptor mutation, e.g., a substitution mutation at least one position of the Fc region. Such substitution mutation(s) may be made to amino acid positions in the Fc domain that include, but are not limited to, e.g., 238, 239, 246, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 332, 333, 334, 335, 337, 338, 340, 360, 373, 376, 378, 382, 388, 389, 398, 414, 416, 419, 430, 434, 435, 437, 438 or 439, wherein the numbering of the residues in the Fc region is according to the EU numbering system. In some embodiments, the anti-CD47 antibody comprises a human IgG4 Fc region that comprises two human IgG4 Fc domain monomers, wherein each monomer comprises an S228P substitution (wherein the numbering of the residue is according to the EU numbering system). Additional suitable mutations are well known in the art. Exemplary mutations are set forth in, e.g., U.S. Pat. No. 7,332,581.

Pharmaceutical Formulations and Administration Thereof

The anti-CD47 antibodies (or immunologically active fragments thereof) provided herein can be formulated with pharmaceutically acceptable carriers or excipients so that they are suitable for administration to a subject in need thereof (e.g., a mammal such as a human). Suitable formulations of the antibodies are obtained by mixing an antibody (or an immunologically active fragment thereof) having the desired degree of purity with optional pharmaceutically acceptable carriers, buffers, excipients or stabilizers (Remington's Pharmaceutical Sciences 16th edition, Osol, A. Ed. (1980)), in the form of lyophilized formulations or aqueous solutions. Pharmaceutically acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propylparaben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

The anti-CD47 antibodies (or immunologically active fragments thereof) provided herein can also be formulated as immunoliposomes. Liposomes containing the antibody are prepared by methods known in the art, such as described in Epstein et al., PNAS USA, 82: 3688 (1985); Hwang et al., PNAS USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martinet al., J. Biol. Chem., 257: 286-288 (1982) via a disulfide-interchange reaction. An anti-neoplastic agent, a growth inhibitory agent, or a chemotherapeutic agent (such as doxorubicin) is optionally also contained within the liposome. See, Gabizon et al., J. National Cancer Inst., 81(19): 1484 (1989).

The active ingredients containing CD47 antibodies may also be entrapped in microcapsule prepared, e.g., by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

A pharmaceutical formulation comprising an anti-CD47 antibody (or immunologically active fragment thereof) provided herein may also contain more than one active compound as necessary for the particular indication being treated, preferably those with complementary activities that do not adversely affect each other. For example, it may be desirable to provide an anti-neoplastic agent, a growth inhibitory agent, a cytotoxic agent, or a chemotherapeutic agent in addition to an anti-CD47 antibody (or immunologically active fragment thereof) provided herein. Such molecules are suitably present in combination in amounts that are effective for the purpose intended. The effective amount of such other agents depends on the amount of antibody present in the formulation, the type of disease or disorder or treatment, and other factors discussed above. The effective amount of such other agents depends on the amount of antibody present in the formulation, the type of disease or disorder or treatment, and other factors discussed above. These are generally used in the same dosages and with administration routes as described herein or about from 1 to 99% of the heretofore employed dosages.

In some embodiments, an antibody of the present disclosure is lyophilized. Such lyophilized formulations may be reconstituted with a suitable diluent to a high protein concentration, and the reconstituted formulation may be administered to a mammal (such as a human).

In certain embodiments, the pharmaceutical formulations to be used for in vivo administration are sterile. This is readily accomplished by, e.g., filtering a solution comprising an anti-CD47 antibody (or immunologically active fragment thereof) provided herein through sterile filtration membranes.

The therapeutic dose of an anti-CD47 antibody described herein may be formulated as a dose of at least about 0.01 μg/kg body weight, at least about 0.05 μg/kg body weight; at least about 0.1 μg/kg body weight, at least about 0.5 μg/kg body weight, at least about 1 μg/kg body weight, at least about 2.5 μg/kg body weight, at least about 5 μg/kg body weight, and not more than about 100 Kg/kg body weight. It will be understood by one of skill in the art that such guidelines will be adjusted for the molecular weight of the active agent, e.g. in the use of antibody fragments, or in the use of antibody conjugates. The dosage may also be varied for localized administration, e.g. intranasal, inhalation, etc., or for systemic administration, e.g., intraperitoneal (I.P.), intravenous (I.V.), intradermal (I.D.), intramuscular (I.M.), and the like.

A CD47 antibody or pharmaceutical composition of this invention can be administered by any suitable means, including parenteral, subcutaneous, intraperitoneal, intrapulmonary, and intranasal. Parenteral infusions include intramuscular, intravenous, intraarterial, intraperitoneal, or subcutaneous administration. In addition, the anti-CD47 antibody is suitably administered by pulse infusion, particularly with declining doses of the antibody.

For the prevention or treatment of disease, the appropriate dosage of antibody will depend on the type of disease to be treated, as defined above, the severity and course of the disease, whether the antibody is administered for preventive purposes, previous therapy, the patient's clinical history and response to the antibody, and the discretion of the attending physician. The antibody is suitably administered to the patient at one time or over a series of treatments.

Methods of Detection and/or Diagnosis

The anti-CD47 antibodies (or immunologically active fragments thereof) provided herein can be used in methods of detection and diagnosis. The presence and/or amount of amount of CD47 (e.g., hCD47) protein in a sample (e.g., biological sample, such as a tissue sample) from a subject can be determined qualitatively and/or quantitatively using an antibody described herein. In certain embodiments, a method of detecting presence and/or amount of amount of CD47 protein comprises contacting the biological sample with an anti-CD47 antibody described herein under conditions permissive for binding of the antibody to CD47, and detecting whether a complex is formed between the antibody and CD47. Such method may be an in vitro or in vivo method. In one embodiment, the method is used to select subjects eligible for therapy with an anti-CD47 antibody. In dome embodiments, the sample is obtained from the subject prior to the subject's being treated with an anti-CD47 antibody. In some embodiments, the tissue sample is formalin fixed and paraffin embedded, archival, fresh or frozen. In some embodiments, the presence and/or amount of CD47 in a first sample is increased or elevated as compared to presence and/or amount of CD47 in a second sample. In certain embodiments, the presence and/or amount of CD47 in a first sample is decreased or reduced as compared to the presence and/or amount of CD47 in a second sample. In certain embodiments, the second sample is a reference sample, reference cell, reference tissue, control sample, control cell, or control tissue. The presence and/or amount of CD47 in a sample can be analyzed by a number of methodologies, many of which are known in the art and understood by the skilled artisan, including, but not limited to, immunohistochemistry (IHC), Western blot analysis, immunoprecipitation, molecular binding assays, ELISA, ELIFA, fluorescence activated cell sorting (FACS), MassARRAY, proteomics, biochemical enzymatic activity assays. Multiplexed immunoassays such as those available from Rules Based Medicine or Meso Scale Discovery (“MSD”) may also be used. Detecting the presence and/or amount of CD47 (e.g., hCD47) protein in a sample (e.g., biological sample, such as a tissue sample) from a subject can be performed in combination with additional techniques such as morphological staining and/or fluorescence in-situ hybridization.

In some embodiments, CD47 expression is evaluated on a tumor or in tumor sample, e.g., relative to a sample of non-cancerous tissue. As used herein, a tumor or tumor sample may encompass part or all of the tumor area occupied by tumor cells. In some embodiments, a tumor or tumor sample may further encompass tumor area occupied by tumor associated intratumoral cells and/or tumor associated stroma (e.g., contiguous peri-tumoral desmoplastic stroma). In some embodiments, CD47 expression is evaluated on tumor cells. In some embodiments, the sample is a clinical sample. In some embodiments, the sample is used in a diagnostic assay. In some embodiments, the sample is obtained from a primary or metastatic tumor. Tissue biopsy is often used to obtain a representative piece of tumor tissue. Alternatively, tumor cells can be obtained indirectly in the form of tissues or fluids that are known or thought to contain the tumor cells of interest. For instance, samples for used in the detection methods described herein may be obtained, without limitation, by resection, bronchoscopy, fine needle aspiration, bronchial brushings, or from sputum, pleural fluid, cerebrospinal fluid, blood, serum, and urine. The same techniques discussed above for detection of target genes or gene products in cancerous samples can be applied to other body samples. Cancer cells may be sloughed off from cancer lesions and appear in such body samples. In some embodiments, an early cancer diagnosis can be achieved by screening such body samples for the presence and/or amount of CD47 protein. In some embodiments, the progress of therapy (e.g., therapy with an anti-CD47 antibody) can be monitored more easily by testing such body samples for the presence and/or amount of CD47 protein.

### Methods of Treatment

Provided herein is a method of treating a disease or disorder associated with aberrant CD47 expression (e.g., CD47 overexpression), the method comprising administering an effective amount of an anti-CD47 antibody described herein to a subject in need thereof. In some embodiments, the disease or disorder is cancer.

Treatment of Solid Tumor

In some embodiments, provided is a method of treating solid tumor in a subject that comprises administering to the subject an effective amount of an anti-CD47 antibody to the subject, wherein the anti-CD47 antibody comprises (a) a heavy chain variable (V_{H}) domain that comprises (1) a CDR-H1 comprising RAWMN (SEQ ID NO: 5); (2) a CDR-H2 comprising RIKRKTDGETTDYAAPVKG (SEQ ID NO: 6); and (3) a CDR-H3 comprising SNRAFDI (SEQ ID NO: 7); (b) a light chain variable (V_{L}) domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10). In some embodiments, the VH further comprises a glutamic acid residue (E) at its N-terminus and a serine (S) at its C-terminus. In some embodiments, the anti-CD47 further comprises a human IgG4 Fc region.

In some embodiments, the solid tumor is relapsed solid tumor (e.g., relapsed during or following a prior treatment for solid tumor) and/or refractory solid tumor (e.g., refractory or not responsive to a prior treatment for solid tumor). In some embodiments, “prior treatment” refers to a therapeutic regimen that comprises administration of one or more therapeutic agents. That is, a “prior treatment” for solid tumor may have comprised treatment with a single therapeutic agent or treatment with a combination of therapeutic agents. In some embodiments, the solid tumor is a lung tumor, an ovarian tumor, a colorectal tumor, a pancreatic tumor, a sarcoma tumor, a head and neck tumor, a gastric tumor, a renal tumor, or a skin tumor (e.g., melanoma). In some embodiments, the solid tumor is a metastatic solid tumor.

In some embodiments, the anti-CD47 antibody is administered to the subject at a dose of about 10 mg/kg to about 30 mg/mg, including any of about 10 mg/kg, 20 mg/kg, and 30 mg/kg. In some embodiments, the anti-CD47 antibody is administered to the subject once a week (i.e., qw or q1w). In some embodiments, the anti-CD47 antibody is administered via intravenous (IV) infusion. In some embodiments, the subject does not experience any treatment-related adverse effects (TRAEs) due to treatment with the anti-CD47 antibody. In some embodiments, the subject does not experience any TRAEs greater than Grade 1 or Grade 2. In some embodiments, TRAEs are graded according to the criteria outlined in Common Terminology Criteria for Adverse Events (CTCAE) v See, e.g., ctep(dot)cancer(dot)gov/protocoldevelopment/electronic applications/docs/CTCAE v5 Quick Refe rence_5×7(dot)pdf.

In some embodiments, the subject does not experience significant hematological toxicity due to treatment with the anti-CD47 antibody. In some embodiments, the subject does not experience any hematological toxicity due to treatment with the anti-CD47 antibody. In some embodiments, the hematological toxicity comprises anemia, cytopenia, and/or hemagglutination. In some embodiments, the subject does not require treatment for hematological toxicity during treatment with the anti-CD47 antibody.

In some embodiments, the V_{H} of the anti-CD47 antibody comprises SEQ ID NO: 1, and the VL of the anti-CD47 antibody comprises SEQ ID NO: 2. In some embodiments, the heavy chain of the anti-CD47 antibody comprises SEQ ID NO: 3 and the light chain of the anti-CD47 antibody comprises SEQ ID NO: 4. In some embodiments, the heavy chain of the anti-CD47 antibody comprises SEQ ID NO: 55 and the light chain of the anti-CD47 antibody comprises SEQ ID NO: 4.

Treatment of Non-Hodgkin Lymphoma (NHL)

In some embodiments, provided is a method of treating non-Hodgkin lymphoma (NHL) in a subject, comprising administering to the subject an effective amount of an anti-CD47 antibody and optionally an effective amount of rituximab, wherein the anti-CD47 antibody comprises (a) a heavy chain variable (V_{H}) domain that comprises (1) a CDR-H1 comprising RAWMN (SEQ ID NO: (2) a CDR-H2 comprising RIKRKTDGETTDYAAPVKG (SEQ ID NO: 6); and (3) a CDR-H3 comprising SNRAFDI (SEQ ID NO: 7); (b) a light chain variable (V_{L}) domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10). In some embodiments, the VH further comprises a glutamic acid residue (E) at its N-terminus and a serine (S) at its C-terminus.

In some embodiments, the anti-CD47 further comprises a human IgG4 Fc region. In some embodiments, the NHL is follicular lymphoma (FL), diffuse large B-cell lymphoma (DLBCL), or mantle cell lymphoma (MCL). In some embodiments, the NHL is relapsed NHL (e.g., relapsed during or following a prior treatment for NHL) and/or refractory NHL (e.g., refractory or non-responsive to a prior treatment for NHL). In some embodiments, the subject has undergone at least one prior treatment (e.g., between 2 and 10 prior treatments) for NHL. In some embodiments, “prior treatment” refers to a therapeutic regimen that comprises administration of one or more therapeutic agents. That is, a “prior treatment” for NHL may have comprised treatment with a single therapeutic agent or treatment with a combination of therapeutic agents. In some embodiments, the subject has undergone prior treatment for NHL that comprised an anti-CD20 agent. In some embodiments, the anti-CD20 agent was an anti-CD20 antibody (e.g., without limitation, rituximab, obinutuzumab, and/or ofatumumab). In some embodiments, the subject progressed (e.g., demonstrated NHL disease progression) during or after treatment with the anti-CD20 agent (e.g., as a single agent or in combination with one or more therapeutic agents).

In some embodiments, the anti-CD47 antibody is administered to the subject once a week (i.e., qw or q1w). In some embodiments, the anti-CD47 antibody is administered to the subject once every 7 days. In some embodiments, the anti-CD47 antibody is administered via intravenous (IV) infusion.

In some embodiments, the rituximab is administered to the subject via IV infusion at a dose of 375 mg/m² once a week (qw or q1w) for five weeks, and at a dose of 375 mg/m² once every 4 weeks (e.g., q4w, q28d, or monthly) following the five weeks. In some embodiments, the rituximab is administered according to the directions of the prescribing label (see, e.g., FDA prescribing label at www(dot)accessdata(dot)fda(dot)gov/drugsatfda docs/label/2018/103705s54501b1(dot)pdf and EMA prescribing label at www(dot)ema(dot)europa(dot)eu/en/documents/overview/mabthera-epar-medicine-overview_en.pdf).

In some embodiments, the subject does not experience any treatment-related adverse effects (TRAEs) due to treatment with the anti-CD47 antibody. In some embodiments, the subject does not experience any TRAEs greater than Grade 1 or Grade 2. In some embodiments, TRAEs are graded according to the criteria outlined in Common Terminology Criteria for Adverse Events (CTCAE) v 5.0. See, e.g., ctep(dot)cancer(dot)gov/protocoldevelopment/electronic applications/docs/CTCAE v5 Quick Refe rence_5×7(dot)pdf.

In some embodiments, the subject does not experience significant hematological toxicity due to treatment with the anti-CD47 antibody. In some embodiments, the subject does not experience any hematological toxicity due to treatment with the anti-CD47 antibody. In some embodiments, the hematological toxicity comprises anemia, cytopenia, and/or hemagglutination. In some embodiments, the subject does not require treatment for hematological toxicity during treatment with the anti-CD47 antibody.

In some embodiments, the V_{H} of the anti-CD47 antibody comprises SEQ ID NO: 1, and the V_{L} of the anti-CD47 antibody comprises SEQ ID NO: 2. In some embodiments, the heavy chain of the anti-CD47 antibody comprises SEQ ID NO: 3 and the light chain of the anti-CD47 antibody comprises SEQ ID NO: 4. In some embodiments, the heavy chain of the anti-CD47 antibody comprises SEQ ID NO: 55 and the light chain of the anti-CD47 antibody comprises SEQ ID NO: 4. In some embodiments, the anti-CD47 antibody is lemzoparlimab.

### Articles of Manufacture and Kits

Provided is an article of manufacture comprising materials useful for the treatment of CD47-associated disease, e.g., a CD47-expressing (such as CD47-overexpressing) cancer, e.g., solid tumor cancer (such as lung cancer, ovarian cancer, colorectal cancer, pancreatic cancer, sarcoma cancer, head and neck cancer, gastric cancer, renal cancer, or skin cancer, etc.) or hematological cancer, e.g., Non-Hodgkin lymphoma (such as diffuse large B-cell lymphoma (DLBCL), follicular lymphoma (FL), mantle cell lymphoma (MCL), etc.). In certain embodiments, the article of manufacture or kit comprises a container containing one or more of the anti-CD47 antibodies (or immunologically active fragments thereof) or the compositions described herein. In certain embodiments, the article of manufacture or kit comprises a container containing nucleic acid(s) encoding one (or more) of the anti-CD47 antibodies (or immunologically active fragments thereof) or the compositions described herein. In some embodiments, the kit includes a cell of cell line that produces an anti-CD47 antibody (or immunologically active fragment thereof) as described herein. In some embodiments, the kit includes one or more positive controls, for example CD47 (or fragments thereof) or CD47⁺ cells. In some embodiments, the kit includes negative controls, for example a surface or solution that is substantially free of CD47, or a cell that does not express CD47.

In certain embodiments, the article of manufacture or kit comprises a container and a label or package insert on or associated with the container. Suitable containers include, for example, bottles, vials, syringes, IV solution bags, test tubes, etc. The containers may be formed from a variety of materials such as glass or plastic. The container holds a composition which is by itself or combined with another composition effective for treating, preventing and/or diagnosing CD47-associated disease or disorder, e.g., cancer, such as solid tumor cancer (e.g., lung cancer, ovarian cancer, colorectal cancer, pancreatic cancer, sarcoma cancer, head and neck cancer, gastric cancer, renal cancer, or skin cancer, etc.) or a hematological cancer (e.g., Non-Hodgkin lymphoma (NHL) such as diffuse large B-cell lymphoma (DLBCL), follicular lymphoma (FL), mantle cell lymphoma (MCL), etc.). The container may have a sterile access port (for example the container may be an intravenous solution bag or a vial having a stopper pierceable by a hypodermic injection needle). At least one agent in the composition is an anti-CD47 antibody (or immunologically active fragment thereof) described herein. In some embodiments, the label or package insert indicates that the composition is used for treating a CD47-associated disease or disorder (e.g., cancer, such as solid tumor cancer (e.g., lung cancer, ovarian cancer, colorectal cancer, pancreatic cancer, sarcoma cancer, head and neck cancer, gastric cancer, renal cancer, or skin cancer, etc.) or a hematological cancer (e.g., Non-Hodgkin lymphoma (NHL) such as diffuse large B-cell lymphoma (DLBCL), follicular lymphoma (FL), mantle cell lymphoma (MCL), etc.). In some embodiments, the label or package insert indicates that the composition is for use in treating solid tumor, such as relapsed and/or refractory solid tumor (e.g., lung, ovarian, colorectal, pancreatic, sarcoma, head and neck, gastric, renal or skin tumor). In some embodiments, the label or package insert indicates that the composition is for use in combination with rituximab for the treatment of non-Hodgkin lymphoma (NHL), such as relapsed and/or refractory NHL in a subject, e.g., a subject who has undergone at least one prior treatment for NHL, such as treatment with an anti-CD20 agent.

Moreover, the article of manufacture or kit may comprise (a) a first container with a composition contained therein, wherein the composition comprises an anti-CD47 antibody (or immunologically active fragment thereof) described herein; and (b) a second container with a composition contained therein, wherein the composition comprises a further cytotoxic or otherwise therapeutic agent (e.g., rituximab, wherein the kit is for treatment of NHL). Additionally, the article of manufacture may further comprise an additional container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits are also provided that are useful for various purposes, e.g., for isolation or detection of CD47, e.g., in a tissue sample obtained from a subject, optionally in combination with the articles of manufacture. For isolation and purification of CD47, the kit can contain an anti-CD47 antibody (or fragment thereof) provided herein coupled to beads (e.g., sepharose beads). Kits can be provided which contain the antibodies (or fragments thereof) for detection and quantitation of CD47 in vitro, e.g., in an ELISA or a Western blot. As with the article of manufacture, the kit comprises a container and a label or package insert on or associated with the container. For example, the container holds a composition comprising at least one anti-CD47 antibody provided herein. Additional containers may be included that contain, e.g., diluents and buffers, control antibodies. Where the antibody is labeled with an enzyme, the kit will include substrates and cofactors required by the enzyme (e.g., a substrate precursor which provides the detectable chromophore or fluorophore). The relative amounts of the various reagents may be varied widely to provide for concentrations in solution of the reagents which substantially optimize the sensitivity of the assay. Particularly, the reagents may be provided as dry powders, usually lyophilized, including excipients which on dissolution will provide a reagent solution having the appropriate concentration. The label or package insert may provide a description of the composition as well as instructions for the intended in vitro or diagnostic use (e.g., detecting CD47, diagnosing a CD47-related disease or disorder), or monitoring the progress of treatment of a CD47-related disease or disorder).

All publications and patent applications cited in this specification are herein incorporated by reference as if each individual publication or patent application were specifically and individually indicated to be incorporated by reference.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Materials and Methods

### Establishment of Phage Library

CD47 is a 50 kDa membrane receptor that has extracellular N-terminal IgV domain, five transmembrane domains, and a short C-terminal intracellular tail. The human CD47-IgV domain conjugated with human Fc or a biotinylated human CD47-IgV domain (ACROBiosystems) was used as antigen for phage library panning.

The phage library was constructed using phagemid vectors which consisted of the antibody gene fragments that were amplified from spleens or bone marrows of >50 healthy human subjects. The antibody format is single chain variable fragment (scFv, VH+linker+VL). The library size was 1.1×10¹⁰ and the sequence diversity was analyzed as follows. For the 62 clones picked up from the library and further sequenced, 16 sequences were truncated, had a frameshift mutation, or an amber codon; 46 sequences had full length scFv, of which all the HCDR3 sequences were unique. In the 46 full length scFv, 13 sequences had lambda light chains, and 33 sequences had kappa light chain.

### Phage Panning and Clone Selection

To obtain phage clones that specifically bind to the human CD47-IgV domain, two methods for phage panning were used.

1. Phage Library Immunotube Panning Against Human CD47-IgV

In this method, the phage libraries developed as described above were first incubated in casein-coated immunotubes for 2 hours. The human CD47-IgV-Fc fusion protein was used for the first round of panning. Unbound phages were removed by washing with PBST 5-20 times. The bound phages were eluted with freshly prepared 100 mM Triethylamine solution and neutralized by addition a Tris-HCl buffer, to become the first output phage pools. This first output phage pool was rescued through infection of E. Coli TG-1 cells for amplification, followed by the second round of panning using biotinylated human CD47-IgV as antigen. The bound phages were eluted in the same process and became the second output phage pool which was then rescued and then again followed by the third round of panning using human CD47-IgV-Fc fusion protein as antigen. The bound phages then became the third output phage pool and underwent the fourth round of panning using biotinylated human CD47-IgV.

2. Phage Library Solution Panning Against Human CD47-IgV

In this second method, the phage libraries were first incubated in casein-blocked 100 μL streptavdin-magnetic beads to deplete streptavdin bead binders. The streptavidin-magnetic beads and A00084-huIgGl/k were used for negative depletion. The depleted library was rescued, which was followed by the second round of panning using biotinylated human CD47-IgV as antigens and further underwent negative depletion with casein blocked streptavdin-magnetic beads. The unbound phages were removed by washing with PBST 5-20 times. The bound phages were eluted with a freshly prepared 100 mM Triethylamine solution, neutralized by addition of a Tris-HCl buffer, and then rescued, which was followed by the third round of panning using human CD47-IgV-Fc fusion protein and depleted with AG0084-hulgGl/k. The bound phages then become the third output phage pool and underwent the fourth round of panning using biotinylated human CD47-IgV and negative depletion with casein blocked streptavdin-magnetic beads.

After this process, multiple phage clones that specifically bound to the human CD47-IgV domain were obtained and enriched. The phage clones were then diluted and plated to grow at 37° C. for 8 hours and captured by anti-kappa antibody-coated filter overnight. Biotinylated human CD47-IgV (50 nM) and NeutrAvidin-AP conjugate (1:1000 dilution) were applied to the filter to detect the positively bound phage clones. Positive phage plaques were picked and eluted into 100 μL of phage elution buffer. About 10-15 μL, eluted phages were used to infect 1 mL XL1 blue cells to make high titer phage (HT) for Phage single point ELISA (SPE). The positive single clones picked from the filer lift were subjected to the binding of human CD47-IgV-Fc fusion protein and biotinylated human CD47-IgV domain protein. These positive single clones were also sequenced for their VH and VL genes. All the positive hits with unique VH and VL genes were cloned into expression vectors pFUSE2ss-CLIg-hk (light chain, InvivoGen, Cat No. pfuse2ss-hclk) and pFUSEss-CHIg-hG1 (heavy chain, InvivoGen, Cat No. pfusess-hchg1). The antibodies were expressed in HEK293 cells and purified by Protein A Plus Agarose.

### Affinity Maturation of Anti-CD47 Antibodies

The binding affinity of the CD47 antibodies obtained as described above can be improved by in vitro affinity maturation, e.g., by site-specific randomized mutation, which resulted in mutated sequences that are also within the scope of this invention.

For example, based on BiaCore analysis, analysis of the CDR sequence of heavy chain and light chain of a CD47 antibody may identify several residues in HCDR1 and LCDR1 regions that could be randomized/mutated. Therefore, the random mutagenesis libraries can be constructed and introduced into the specific residues to generate a variety of new sequences. The CDR mutagenesis libraries are panned using biotinylated soluble CD47 ECD in solution phase under the equilibrium condition. After multiple rounds of panning with reduced antigen concentration, enriched output binders are selected for the binding ELISA test and subsequent converted into full IgGs which are subjected to the BiaCore analysis to specifically select for the off-rate improved sequence. Through this screening process, additional antibody molecules of this invention can be constructed for overall best properties for clinical applications.

### Example 1. ELISA Screening of Phage Clones Binding to Recombinant CD47-ECD

### (Extracellular Domain)

Recombinant human CD47 IgV-Fc fusion protein (Acrobiosystems) was coated at 2 μg/mL in phosphate buffer saline (PBS) onto microtiter plates for 2 hours at the room temperature (RT). After coating of antigen, the wells were blocked with PBS/0.05% Tween (PBST) with 1% BSA for 1 hour at RT. After washing of the wells with PBST, purified phages from single clones were added to the wells and incubated for 1 hour at RT. For detection of the binding phage clones, horseradish peroxidase (HRP) conjugated secondary antibodies against M13 (Jackson Immuno Research) were added, followed by the addition of fluorogenic substrates (Roche). Between all incubation steps, the wells of the plate were washed with PBST three times. Fluorescence was measured in a TECAN Spectrafluor plate reader. The positive phage clones were selected for sequencing of the heavy chain and light chain genes.

The CD47 antibodies obtained as described above showed good binding activities for recombinant human CD47 IgV-Fc fusion protein.

### Example 2. ELISA Analysis of Antibodies Blocking the Interaction of CD47 and SIRPα

Recombinant human CD47 IgV/mouse Fc fusion protein or biotinylated CD47 IgV protein (Acrobiosystems) was coated at 1 μg/mL in PBS onto microtiter plates for 2 hours at RT. After coating with antigen, the wells were blocked with PBS/0.05% Tween (PBST) with 1% BSA for 1 hour at RT. After washing of the wells with PBST, the antibodies diluted in PBS were added to the wells (5 μg/mL) and incubated for 1 hour at RT. For detection of the binding by antibodies, the HRP conjugated secondary antibodies against human Fc (Jackson Immuno Research) were added, followed by the addition of fluorogenic substrates (Roche). Between all incubation steps, the wells of the plate were washed with PBST three times. Fluorescence was measured in a TECAN Spectrafluor plate reader.

The CD47 antibodies A1A and B2B) showed good binding activities for recombinant human CD47-Fc fusion protein and biotinylated CD47 protein. Example 3. ELISA Analysis of Antibodies Blocking the Interaction of CD47 and SIRPα

Recombinant hCD47 IgV-Fc fusion protein (Acrobiosystems) was coated at 1 μg/mL in PBS onto microtiter plates for 16 hours at 4° C. After blocking for 1 hour with 1% BSA in PBST at RT, 1 μg/ml of SIRPα-His protein was added either in the absence or presence of anti-CD47 antibodies (10 μg/mL) at RT for 1 hour. Plates were subsequently washed three times and incubated with an HRP-conjugated anti-His secondary antibody for 1 hour at RT. After washing, the TMB solution was added to each well for 30 minutes and the reaction was stopped with 2.0 M H₂SO₄, and OD was measured at 490 nm.

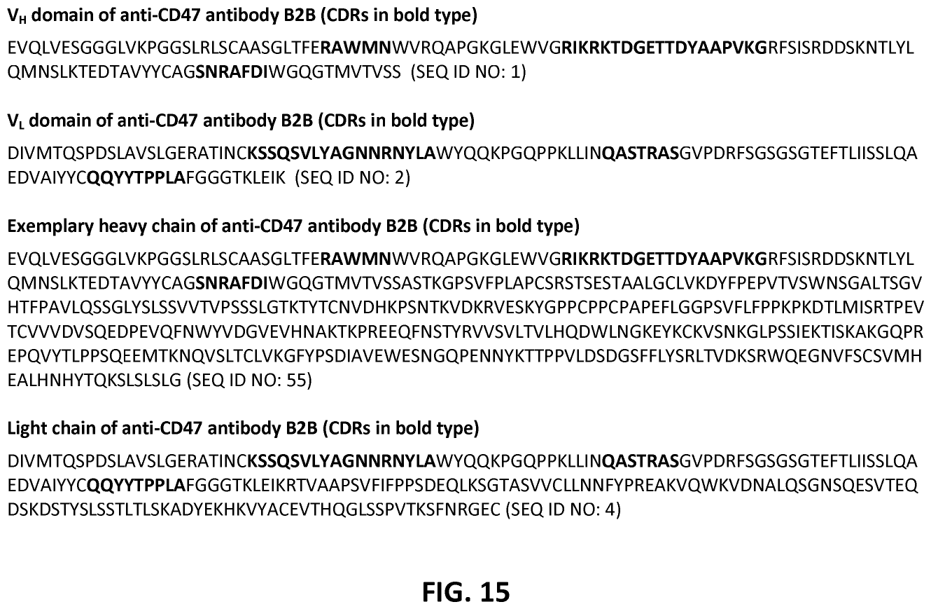

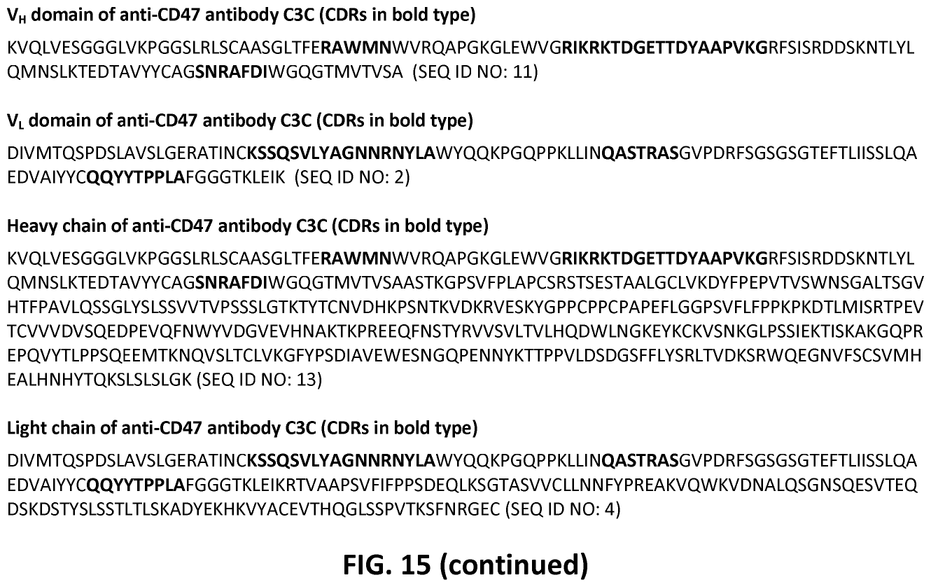

CD47 antibodies A1A and B2B effectively blocked binding of CD47 to SIRPα. The amino acid sequences of the V_{H} domain, VL domain, heavy chain, and light chain of B2B and A1A are shown in FIG. 15.

### Example 4. Dose-Dependent Response of Anti-CD47 Antibodies Binding to Monomeric CD47-ECD

After direct binding and competition screening, the anti-CD47 antibody B2B was selected for this test, in comparison with two known reference anti-CD47 antibodies, i.e., F59 and 2A1. Biotinylated CD47 protein (Acrobiosystems) was coated at 1 μg/mL in PBS onto microtiter plates for 2 hours at RT. After coating of antigen, the wells were blocked with PBS/0.05% Tween (PBST) with 1% BSA for 1 hour at RT. After washing of the wells with PBST, different concentrations of anti-CD47 antibodies were added to the wells and incubated for 1 hour at RT. For detection of the binding of the antibodies to CD47, the HRP conjugated secondary antibodies against human Fc (Jackson Immuno Research) were added followed by the addition of fluorogenic substrates (Roche). Between all incubation steps, the wells of the plate were washed with PBST three times. Fluorescence was measured in a TECAN Spectrafluor plate reader.

Reference antibodies 5F9 and 2A1 was produced according to the sequence of Hu5F9 and CC-90002 as disclosed by researchers at Stanford University, Inhibrx LLC, and Celgene Corp. (see, e.g., U.S. Pat. No. 9,017,675 B2, U.S. Pat. Nos. 9,382,320, 9,221,908, US Pat. Application Pub. No. 2014/0140989 and WO 2016/109415) and used for the same study.

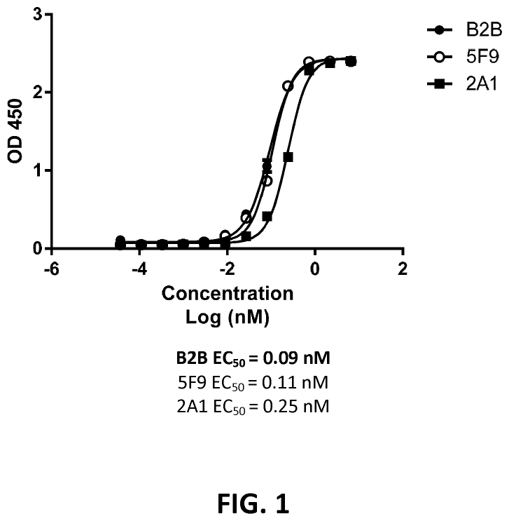

As shown in FIG. 1, anti-CD47 antibody B2B showed binding activities to monomeric CD47-ECD superior to those of 5F9 and 2A1. B2B's EC50 of 0.09 nm was lower than the EC50s of 5F9 (0.11 nM) and 2A1 (0.25 nM).

### Example 5. Dose-Dependent Response of Anti-CD47 Antibodies Binding to Dimeric CD47-ECD

The two anti-CD47 antibodies identified in Example 4 (i.e., A1A and B2B) were also used in this study.

CD47 IgV/mouse Fc fusion protein (Acrobiosystems) was coated at 1 μg/ml in PBS onto microtiter plates for 2 hours at RT. After coating of antigen the wells were blocked with PBS/0.05% Tween (PBST) with 1% BSA for 1 hour at RT. After washing of the wells with PBST, different concentrations of anti-CD47 antibodies were added to the well and incubated for 1 at RT. For detection of the binding antibodies, the HRP conjugated secondary antibodies against human Fc (Jackson Immuno Research) were added followed by the addition of fluorogenic substrates (Roche). Between all incubation steps, the wells of the plate were washed with PBST three times. Fluorescence was measured in a TECAN Spectrafluor plate reader.

Anti-CD47 antibody B2B showed binding activities to dimeric CD47-ECD in a dose-dependent manner.

### Example 6. Dose-Dependent Response of Anti-CD47 Antibodies Blocking the Binding of CD47 to SIRPα

Recombinant CD47-Fc fusion protein (Acrobiosystems) was coated at 1 μg/ml in PBS onto microtiter plates for 16 hours at 4° C. After blocking for 1 hour with 1% BSA in PBST at RT, 1 μg/mL of SIRPα-His protein was added either in the absence or presence of different concentrations of anti-CD47 antibodies at RT for 1 hour. Plates were subsequently washed three times and incubated with an HRP-conjugated anti-His secondary antibody for 1 hour at RT. After washing, the TMB solution was added to each well for 30 min and the reaction was stopped with 2M H₂SO₄, and OD was measured at 490 nm.

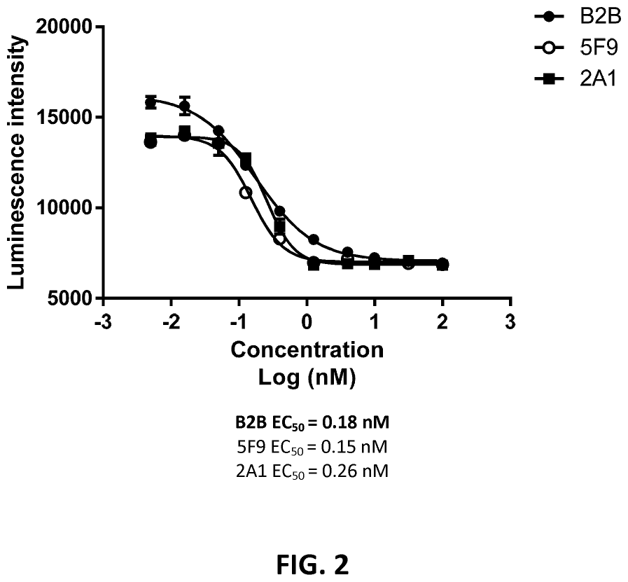

As shown in FIG. 2, anti-CD47 antibody B2B showed activities in blocking the binding of CD47 to SIRPα in a dose-dependent manner, with an EC 50 of 0.18 nM.

### Example 7A. Dose-dependent Response of anti-CD47 Antibodies Binding to CD47⁺ Raji Cells

Raji cells, which endogenously express human CD47 on the surface, were stained with different concentrations of anti-CD47 antibodies at 4° C. for 30 minutes. Then, the cells were washed with PBS three times, followed by incubation with APC-labeled anti-human Fc specific antibody (Invitrogen) at 4° C. for 30 minutes. Binding was measured using a FACSCanto (Becton-Dickinson).

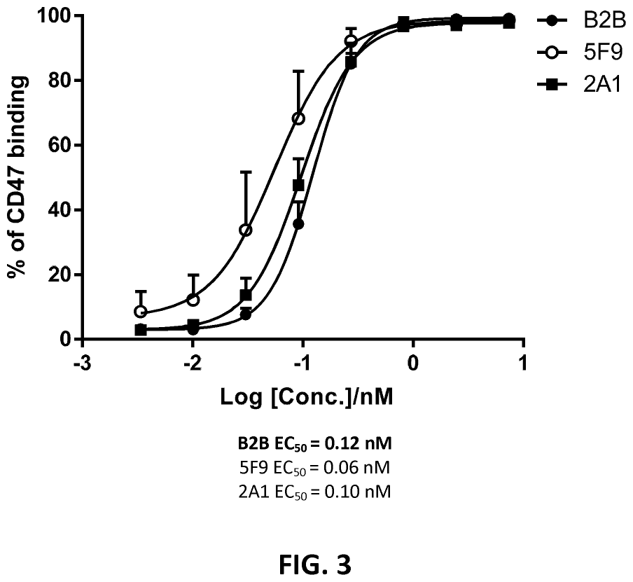

As shown in FIG. 3, the anti-CD47 antibody B2B showed activities in binding to CD47⁺ Raji cells, following the same dose-dependent pattern, with an EC 50 of 0.12 nM.

### Example 7B: Dose-Dependent Response of Anti-CD47 Antibodies Binding to Tumor Cells

Similar studies were conducted with a panel of 12 tumor cell lines across different tumor lineages including both leukemic and solid tumor lineages for evaluating the binding intensity of the antibodies of the present invention.

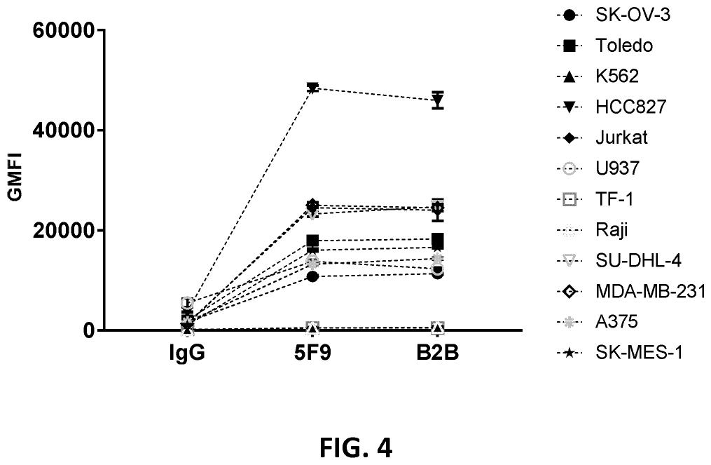

As shown in FIG. 4, the anti-CD47 antibody B2B showed a comparable pattern of binding intensity with 5F9 on the 12 cell lines tested (i.e., SK-OV-3, Toledo, K562, HCC827, Jurkat, U937, TF-1, Raji, SU-DHL-4, MDA-MB-231, A375, and SK-MES-1), which was closely corrected with the phagocytosis pattern of B2B and 5F9 in the same tumor cell lines as discussed below.

### Example 8A. Study of Phagocytosis of Tumor Cells by Human Macrophages

Peripheral blood mononuclear cells (PBMCs) were isolated from human blood, and the monocytes were differentiated into macrophages for 6 days. The monocyte derived macrophages (MDMs) were scraped and re-plated in 24-well dishes and allowed to adhere for 24 hours. Raji cells, which endogenously expresses CD47, were chosen as the target cells and labeled with 1 μM carboxyfluorescein succinimidyl ester (CFSE) for 10 minutes, then added to monocyte derived macrophages (MDMs) at a ratio of 5:1 tumor cells per phagocyte. Anti-CD47 antibodies were then added at various doses. After incubation for 3 hours, non-phagocytosed target cells were washed away with PBS and the remaining phagocytes were scraped off, stained with macrophage marker CD14 antibody, and analyzed by flow cytometry. Phagocytosis was measured by gating on CD14⁺ cells and then assessing the percent of CFSE cells.

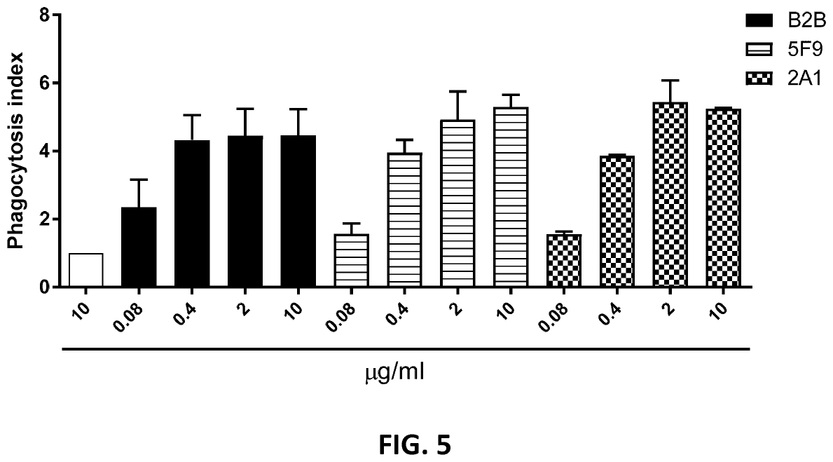

As shown in FIG. 5, the anti-CD47 antibody B2B showed similar activities in promoting phagocytosis of tumor cells by human macrophage as those of anti-CD47 antibodies 5F9 and 2A1.

### Example 8B. Further Study of Phagocytosis of Tumor Cells

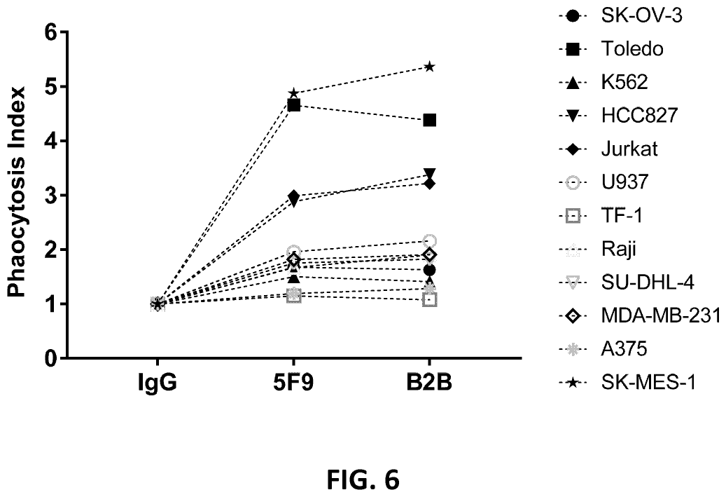

Similar studies were conducted with a panel of 12 tumor cell lines across different tumor lineages including both leukemic and solid tumor lineages for evaluating the phagocytosis intensity of the antibodies of the present invention. As shown in FIG. 6, the anti-CD47 antibody B2B showed comparable activities in promoting phagocytosis of SK-OV-3, Toledo, K562, HCC827, Jurkat, U937, TF-1, Raji, SU-DHL-4, MDA-MB-231, A375, and SK-MES-1 cells as those of anti-CD47 antibody 5F9.

### Example 9. RBC-Sparing Property in Red Blood Cell (RBC) Agglutination Assay

Human RBCs were diluted to 10% in PBS and incubated at 37° C. for 2 hours with a titration of anti-CD47 antibodies in the wells of a round bottom 96-well plate. Evidence of hemagglutination is demonstrated by the presence of non-settled RBCs, appearing as a haze compared to a punctuate red dot of non-hemagglutinated RBCs.

The anti-CD47 antibody B2B resulted in no RBC agglutination at the tested concentrations up to 30 μg/μL or even up to 150 μg/mL.

### Example 10. RBC Binding Assay

Binding of CD47 antibodies against human RBCs was examined by flow cytometry. Human RBCs were incubated with CD47 antibodies (10 μg/mL) at 4° C. for 1 hour, followed by the addition of Allophycocyanin (APC) conjugated secondary antibody at 4° C. for 30 minutes.

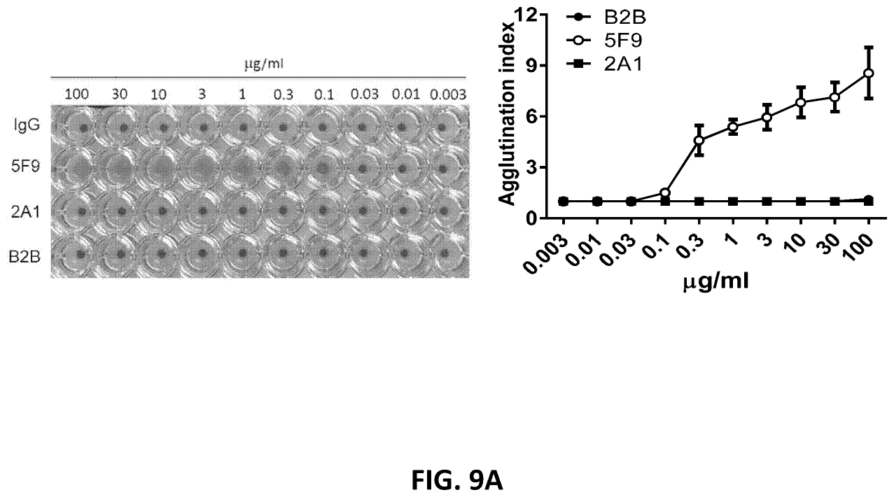

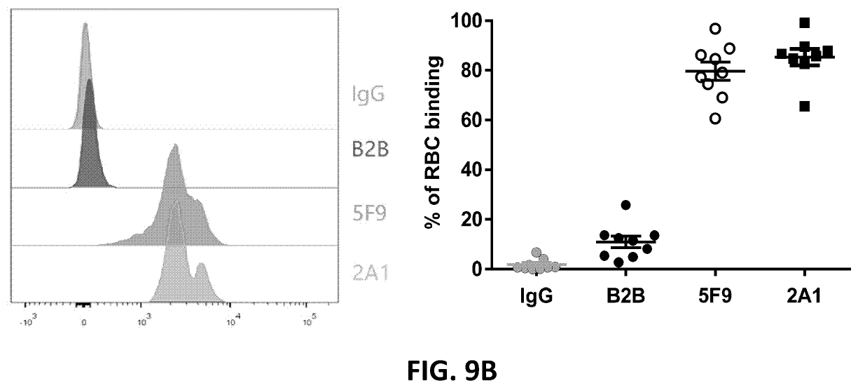

As shown in FIG. 9A, the anti-CD47 antibody B2B resulted in only very low RBC binding (usually below 15%) at the tested concentrations, whereas the reference anti-CD47 antibody showed much higher RBC binding (usually between 70-90%) at the same concentrations.

### Example 11. RBC Agglutination Assay

RBCs were collected from six male and six female healthy individuals for the analysis of RBC agglutination by the addition of CD47 antibodies.

As shown in FIG. 9B, anti-CD47 antibody B2B showed no RBC agglutination, but the reference anti-CD47 antibody 5F9 and 2A1, caused significant agglutination.

### Example 12. Platelet Binding Assay

Binding of CD47 antibodies of this invention against human platelets was examined by flow cytometry. Human peripheral whole blood was incubated with test CD47 antibodies described herein (at 10 μg/mL) or SIRPα-Ig fusion, and CD61 was stained as a cell surface marker for platelets. The binding of anti-CD47 antibodies or SIRPα-Ig fusion was measured by gating on the CD61 positive population (platelet) and further examining the percentages of CD47 or SIRPα-Ig fusion binding.

The anti-CD47 antibody B2B did not appreciably bind to human platelets whereas the SIRPα-Ig fusion protein did.

### Example 13. Phagocytosis of Primary Human Acute Myeloid Leukemia Cells Induced by CD47 Antibodies

Primary peripheral blood mononuclear cells (PBMCs) from a human acute myeloid leukemia (AML) patient were labeled with 1 μM carboxyfluorescein succinimidyl ester (CFSE) for 10 minutes, then added to monocyte derived macrophages (MDMs) at a ratio of 5:1 tumor cells per phagocyte and the indicated CD47 antibodies was added at various concentrations. After a 3 hour incubation, non-phagocytosed target cells were washed away with PBS and the remaining phagocytes were scraped off, stained with a CD14 antibody, and analyzed by flow cytometry. Phagocytosis was measured by gating on CD14⁺ cells and then assessing the percentage of CFSE cells. Phagocytosis was measured as previously described.

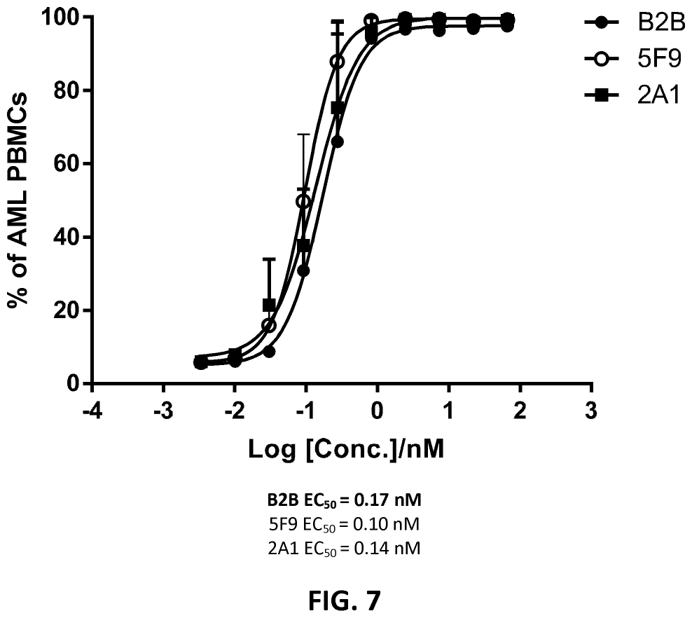

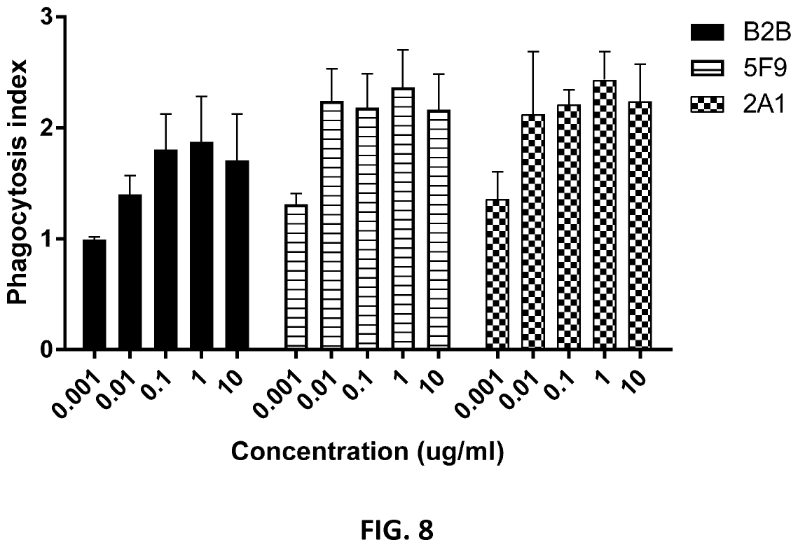

As shown in FIG. 7 and FIG. 8, respectively, the anti-CD47 antibody B2B showed significant AML binding capabilities (greater than 95%) and phagocytosis capabilities (at least 36%), comparable to the activities of reference anti-CD47 antibody 5F9.

### Example 14. In Vivo Efficacy of Anti-CD47 Antibody B2B in a Luciferase-Raji Cell-Line Derived Xenograft (CDX) Model

NOD scid gamma (NSG) mice were engrafted with Raji Luc-EGFP (enhanced green fluorescent protein) at a concentration of 1 million cells/mouse via tail vein injection. The mice were imaged in vivo to determine the level of engraftment five days post engraftment. Treatment using an anti-CD47 antibody B2B, started from the same day at different doses. A control group was given vehicle. All mice were injected every other day via intraperitoneal injection. Mice were imaged in vivo via IVIS Lumina III imaging system on days 0, 4, 7, 11, 14, 18, and 21 after antibody treatment. The tumor growth in the mice was measured by the analysis of bioluminescent radiance through in vivo live imaging system.

In the end of Raji-xenograft study, all the mice were euthanized. The splenocytes from the B2B-treated mice and vehicle-treated mice were isolated and analyzed for the percentage of M1 macrophages (% of CD80 positive in F4/80 positive macrophages) and M2 macrophages (% of CD206 positive in F4/80 positive macrophages) by flow cytometry analysis.

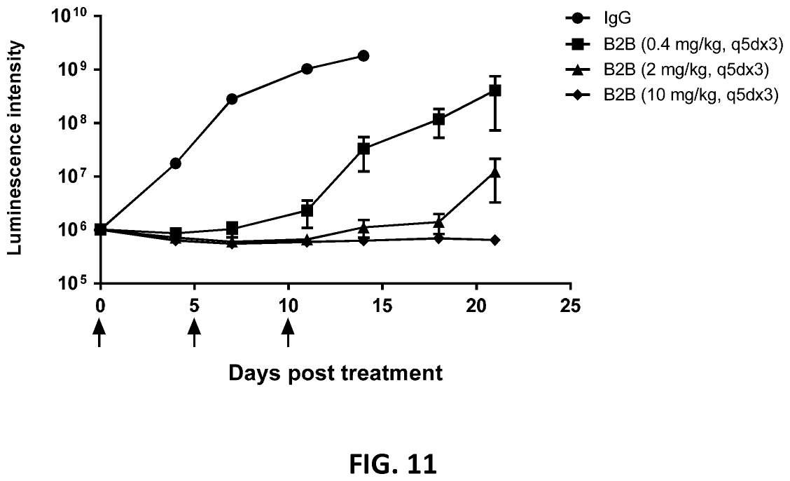

FIG. 11 shows that the luminescence intensity of the mice treated with the anti-CD47 antibody B2B continued to decrease after a treatment of 10 mg/kg but only increase slightly following treatments of lower concentrations. This demonstrated that B2B effectively induced polarization of macrophage in tumor-bearing mice.

### Example 15. Pharmacological Safety Study in Cynomolgus Monkeys

Pilot—Single Dose: Naïve cynomolgus monkeys were intravenously infused with vehicle (n=2), anti-CD47 antibody B2B (n=3, dose=15 mg/kg), or anti-CD47 antibody 5F9 (n=3, dose=15 mg/kg). Hematology (complete blood count or “CBC”) was analyzed within 24 hours after blood collection, twice before anti-CD47 antibody administration and at 3, 6, 10, 14 and 21 days following antibody administration. CBC parameters were examined including erythrocyte count (also known as red blood cell or “RBC”), hemoglobin (or “HGB”), absolute reticulocyte count, and platelet count.

Pilot—Repeat Dose: Similarly, naïve cynomolgus monkeys (n=2) were intravenously injected with the anti-CD47 antibody B2B at a dose of 20 mg/kg. Blood samples from each monkey were collected by venipuncture into tubes with no anticoagulant at different time points. Hematology (CBC) parameters were examined at the indicated time points following the antibody administration. The hematological parameters included erythrocyte count (RBC), Hemoglobin (HGB), platelet counts, and lymphocyte counts. at the indicated time points following the antibody administration.

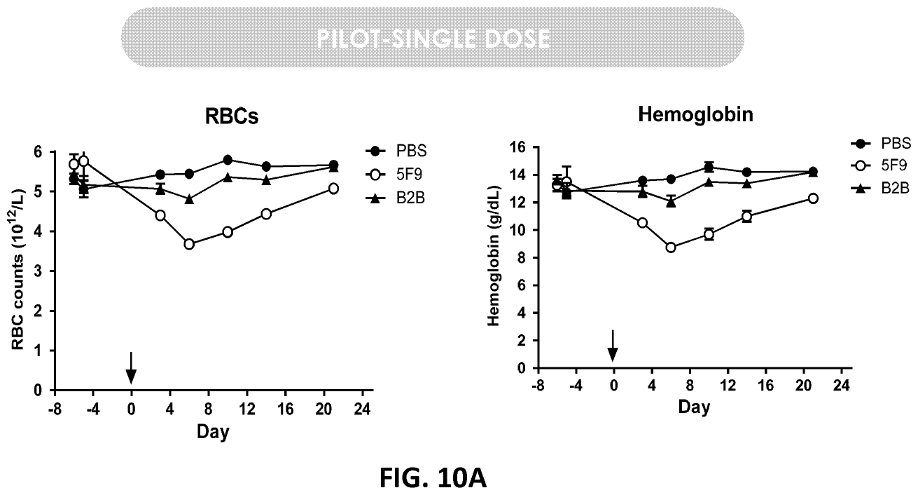

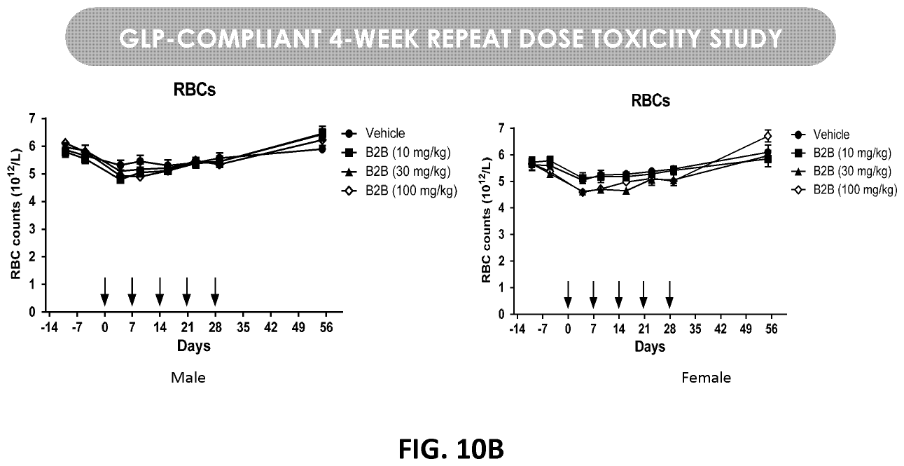

FIG. 10A and FIG. 10B show that the anti-CD47 antibody B2B did not induce significant hematologic changes in cynomolgus monkeys following administration.

A Good Laboratory Practice (GLP)-compliant 4 week repeat-dose intravenous (IV) toxicity study in cynomolgus monkeys was performed as follows. Naïve cynomolgus monkeys were intravenously infused with repeat doses (weekly dosing) of the anti-CD47 antibody B2B at 10 mg/kg, mg/kg, or 100 mg/kg. Hematology (CBC) parameters were examined including erythrocyte count (RBC), Hemoglobin (HGB), platelet counts and lymphocyte counts at the indicated time points. FIG. shows that single dose treatment of B2B had a minimal influence on the level of RBCs and hemoglobin as compared to the treatment of 5F9. FIG. 10B shows that repeated treatments with B2B at different dosage did not significantly affect the RBCs in either male and female cynomolgus monkeys, as compared to vehicle control.

### Example 16. Clinical Study in Patients with Relapsed/Refractory Solid Tumors and Lymphoma

A two-part clinical study was conducted with anti-CD47 antibody B2B in patients with relapsed/refractory malignancy. Part 1 of the clinical study consisted of B2B dose escalation, and Part 2 is a dose expansion study. During the dose escalation (Part 1), patients with relapsed/refractory solid tumors were administered with an intravenous weekly dose (1 mg/kg to 30 mg/kg) of B2B to determine tolerability, safety, pharmacokinetics (PK), pharmacodynamics (PD) and anti-tumor activity based on Response Evaluation Criteria in Solid Tumors (RECIST v1.1) and iRECIST. (See, e.g., Eisenhauer et al. (2009) European J. Cancer. 45:228-247 and Seymour et al. (2017) Lancet Oncol. 18(3): e143-e152.

More specifically, twenty patients with relapsed/refractory solid tumors were assigned to one of five B2B dose escalation cohorts (1, 3, 10, 20 and 30 mg/kg). B2B toxicity was manageable up to 30 mg/kg without any dose-limiting toxicity (DLT) observed. The most common treatment-related adverse events (TRAEs) were anemia (30.0%, n=6), fatigue (25.0%, n=5), infusion-related reactions (20.0%, n=4), and diarrhea (15.0%, n=3). All TRAEs were Grade 1 or 2. A transient, non-dose-dependent average reduction of 1.5 mg/dL (range: 0.4-2.6 mg/dL) in hemoglobin during the first cycle was observed across all cohorts, consistent with the results of pre-clinical good laboratory practice toxicity studies. Laboratory or clinical evidence of hemolysis was not observed in any cohort. Preliminary results indicate the pharmacokinetics of B2B appeared to be linear at mid- to high-dose levels following a single dose. CD47 receptor occupancy showed complete saturation on peripheral T cells at peak concentrations of 20 mg/kg and above.

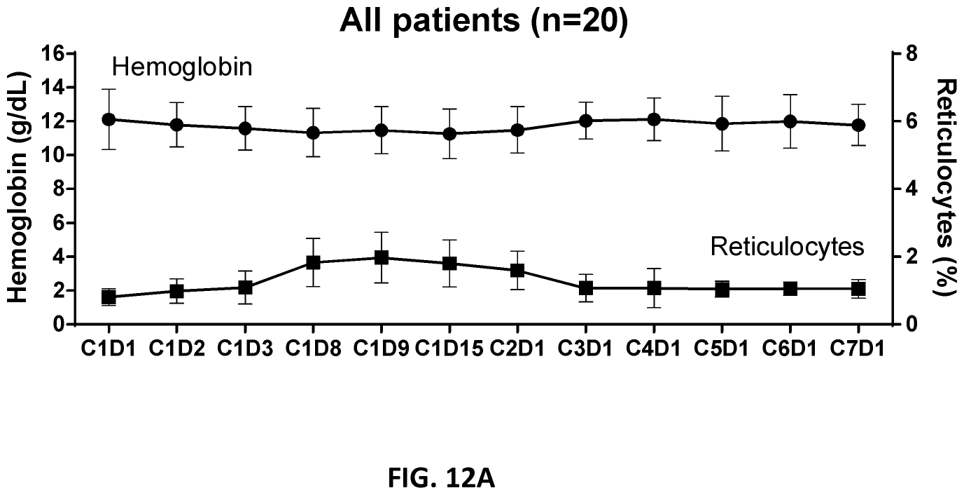

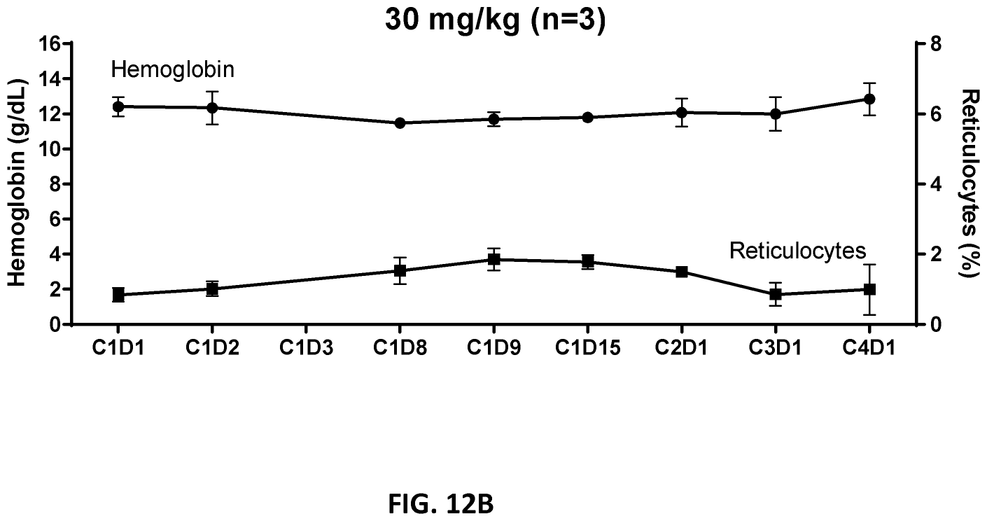

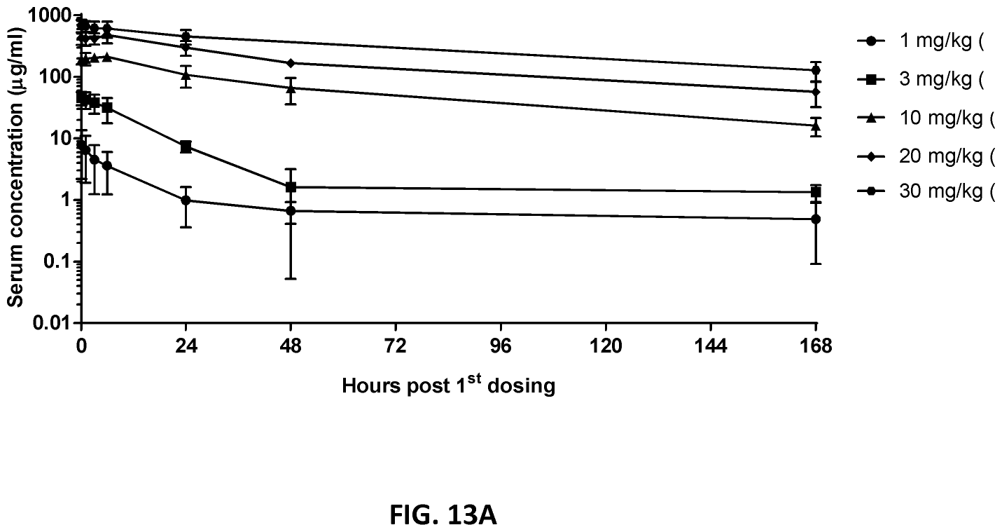

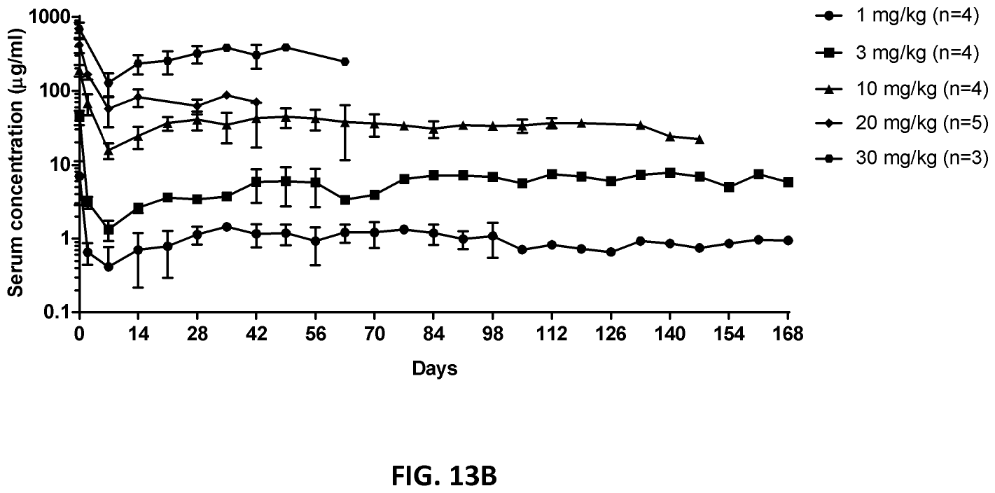

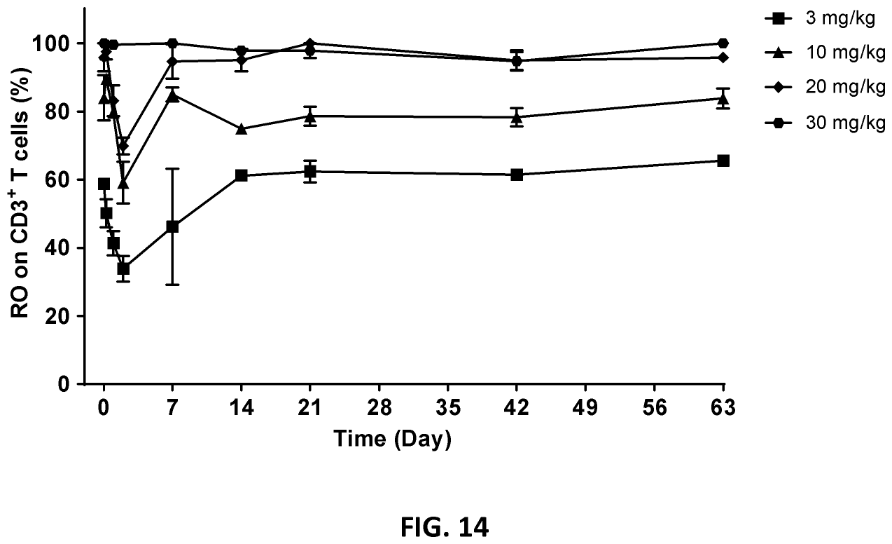

As shown in FIGS. 12-14, the anti-CD47 antibody of this invention (i.e., B2B) appeared to be safe up to 30 mg/kg with favorable pharmacokinetic (PK) and pharmacodynamic (PD) characteristics in patients with relapsed/refractory solid tumors. TRAEs greater than Grade 2 had been observed.

### Example 17. Anti-CD47 Antibody Production

cDNAs the encoding heavy chain (SEQ ID NO. 3) and the light chain (SEQ ID NO. 4) of antibody B2B were synthesized and cloned into in house vector PIM4.0, respectively. The vectors comprising said cDNAs were then stably co-transfected into CHO-K1 host cells for antibody production. A reference cell line, which was stably transfected with vectors comprising nucleic acids encoding the antibody C3C heavy chain (SEQ ID NO. 7) and light chain (SEQ ID NO. 8) was developed in parallel.

Following mini pool selection and a series of expansions, CHO-K1 cells expressing B2B or C3C were respectively inoculated into ActiPro medium at a density of 5×10⁵ cells/mL in 50 mL spin tubes. Three batches, each with working volumes of 20 mL, were prepared for each antibody. Cell Boost7a (CB7a) and Cell Boost 7b (CB7b) (10:1) were used as feed medium. Briefly, 0%˜5.0% CB7a and 0%˜0.5% CB7a were added on day 3, day 6, day 8, day 10 and day 12. The feeding percentage and feed day were adjusted based on the growth and metabolic profiles. Glucose was also added into the cultures. The fed-batch cultures were incubated in the Kuhner shaker (36.5° C., 75% humidity, 6% CO2, 225 RPM). Culture temperature was shifted to 31° C. either (a) when viable cell density (VCD) reached about 16×10⁶ cells/mL or (b) on day 7, whichever came first.

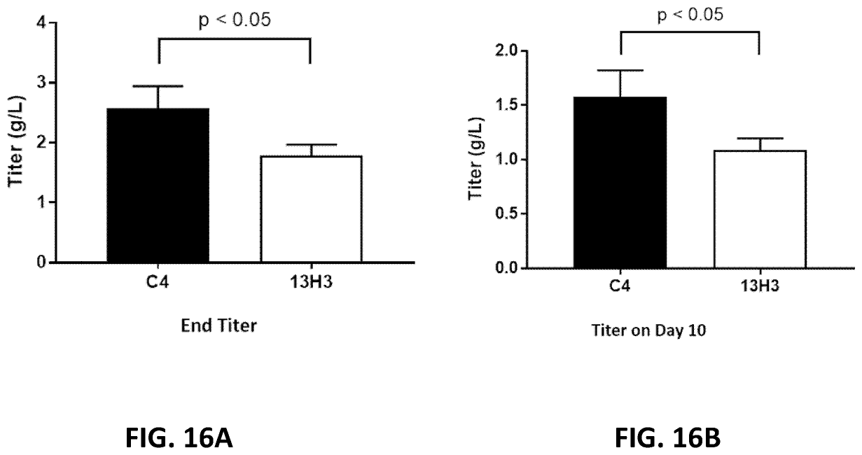

Fed-batch cultures from each batch were harvested on day 10 and day 14. Supernatants from harvested cultures were measured for titer by Protein A-HPLC. The titer of each batch is as shown in Table 1 and FIGS. 16A and 16B.

TABLE 1
Production Titer of Fed-Batch Cultures

| | End Titer | Mean End | Titer on | Mean Run Titer |
|---|---|---|---|---|
| | on Day | Run Titer | Day 10 | on Day |
| Pool ID | 14 (g/L) | (g/L) | (g/L) | 10 (g/L) |
| B2B-batch 1 | 2.90 | 2.58 ± 0.37 | 1.74 | 1.58 ± 0.24 |
| B2B-batch 2 | 2.65 | | 1.70 | |
| B2B-batch 3 | 2.18 | | 1.30 | |
| C3C-batch 1 | 1.99 | 1.79 ± 0.18 | 1.18 | 1.09 ± 0.11 |
| C3C-batch 2 | 1.74 | | 0.97 | |
| C3C-batch 3 | 1.65 | | 1.12 | |

Supernatants of fed-batch cultures of the top two pools for each antibody were further purified by one-step Protein A purification. The Protein A purified antibodies were then subject to quality analysis by size exclusion chromatography (SEC) as shown in Table 2, and capillary electrophoresis (CE) as shown in Table 3.

TABLE 2
SEC Profiles of Top Stable Pools

| Pool ID | Main Peak (%) | HMW Peak (%) | LMW Peak (%) |
|---|---|---|---|
| B2B-batch 3 | 97.5 | 2.4 | 0.1 |
| B2B-batch 2 | 97.7 | 2.2 | 0.1 |
| C3C-batch 1 | 97.8 | 1.6 | 0.6 |
| C3C-batch 2 | 98.2 | 1.3 | 0.5 |

TABLE 3
CE Profiles of Top Stable Pools

| Pool ID | Main Peak (%) | LC + HC (%) | LC (%) | HC (%) |
|---|---|---|---|---|
| B2B-batch 3 | 95.9 | 97.7 | 33.3 | 64.4 |
| B2B-batch 2 | 95.4 | 98.2 | 33.4 | 64.8 |
| C3C-batch 1 | 93.2 | 97.7 | 33.0 | 64.7 |
| C3C-batch 2 | 93.3 | 98.2 | 33.6 | 64.6 |

The results above demonstrate that CHO-K1 cells expressing antibody B2B yielded significantly higher mean titer of antibody on both day 10 and the end day (e.g., day 14), as compared to CHO-K1 cells expressing antibody C3C. Table 1 shows superior production of anti B2B antibody, from CHO-K1 cells, as compared to antibody C3C. Tables 2 and 3 show that the product quality of antibody B2B expressed by CHO-K1 cells is comparable that of antibody C2C expressed by CHO-K1 cells.

### Example 18: Initial Monotherapy Results from a First-In-Patient Study of Lemzoparlimab, a Differentiated Anti-CD47 Antibody, in Subjects with Relapsed/Refractory Malignancies

### Background

CD47 is expressed on most cancers. Blockade of the interaction between CD47 and SIRPα results in the inhibition of the “do not eat me” signal and leads to phagocytosis of tumor cells expressing CD47. Anti-CD47 antibodies as a drug class have emerged as a promising therapy for cancers, which is supported by the initial clinical data in patients with lymphoma (see, e.g., Example 22) and leukemia. However, CD47 is also naturally expressed on red blood cells (RBC). Initial clinical and pre-clinical studies have shown that various therapeutic anti-CD47 antibodies can cause hematologic toxicities, namely severe anemia or thrombocytopenia.

Lemzoparlimab (also known as TJ011133, TJC4, or B2B) is a novel fully human CD47 antibody of the IgG4 isotype. It is uniquely selected, by design, for minimal interaction with RBC and is highly differentiated from other CD47 antibodies of the same class. Lemzoparlimab induces only minimal and transient reduction in RBC levels in cynomolgus monkeys (see, e.g., FIG. 9B). The RBC-sparing property of lemzoparlimab is attributable mechanistically to its recognition of a unique glyco-epitope of CD47 that is shielded by glycosylation on RBC. Lemzoparlimab retains strong activities for (a) binding to various tumor cell types, (b) tumor phagocytosis in vitro and (c) tumor eradication in mouse xenograft models.

### Methods/Study Design

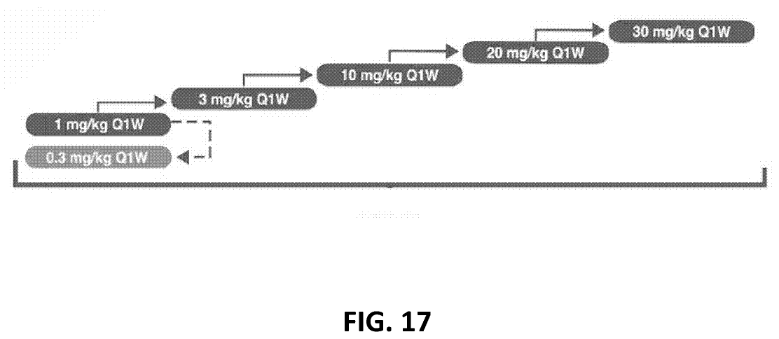

This Example provides preliminary results from a Phase 1 study designed to evaluate the safety, tolerability, maximal tolerable dose (MTD) or maximum administered dose (MAD), pharmacokinetics (PK) and pharmacodynamics (PD), and recommended phase 2 dose (RP2D) of lemzoparlimab in subjects with advanced relapsed or refractory solid tumors and lymphoma. In Part 1 of the study, a single agent dose escalation in a standard 3+3 design was used. See FIG. 17. Lemzoparlimab was administered as weekly IV infusions to patients with advanced relapsed or refractory solid tumors in successive dose cohorts (1, 3, 10, 20 and 30 mg/kg) without any priming dose (e.g. low (−1 mg/kg) weekly dose(s)) commonly used when administering therapeutic anti-CD47 antibodies).

### Results

Baseline Characteristics

Twenty patients with advanced relapsed or refractory solid tumors (i.e., lung, ovarian, colorectal, pancreatic, sarcoma, head and neck, gastric, renal and skin) were enrolled into the monotherapy dose escalation study. Patients' baseline characteristics and the number of patients given 1, 3, 10, 20, or 30 mg/kg lemzoparlimab qw are shown in Table 4:

TABLE 4
Baseline Characteristics

| | | 1 mg/kg | 3 mg/kg | 10 mg/kg | 20 mg/kg | 30 mg/kg | Total | |
|---|---|---|---|---|---|---|---|---|
| | | (N = 4) | (N = 4) | (N = 5) | (N = 6) | (N = 3) | (N = 20) | |
| Age | Median | 69 (63, 76) | 59 (35, 68) | 61 (54, 63) | 59 (53, 75) | 59 (58, 74) | 62 | (35, 76) |
| | (Range) | | | | | | | |
| Sex | Female | 3 | 0 | 3 | 2 | 0 | 8 | (40%) |
| | Male | 1 | 4 | 1 | 3 | 3 | 12 | (60%) |
| Race | African | 0 | 0 | 0 | 0 | 1 | 1 | (5%) |
| | American | | | | | | | |
| | Asian | 0 | 0 | 0 | 1 | 0 | 1 | (5%) |
| | White | 4 | 4 | 3 | 3 | 2 | 18 | (90%) |
| ECOG PS | 0 | 0 | 0 | 1 | 2 | 1 | 4 | (25%) |
| | 1 | 4 | 4 | 3 | 3 | 2 | 16 | (75%) |

Safety

No dose limiting toxicities (DLT) or drug-related serious adverse effects (SAEs) were reported throughout the study. All treatment-associated adverse effects (TAAEs) were either Grade 1 or Grade 2 except one Grade 3 lipase increase. See Table 5 (GR=grade). All toxicities were graded using Common Terminology Criteria for Adverse Events (CTCAE) v 5.0. See, e.g., ctep(dot)cancer(dot)gov/protocoldevelopment/electronic applications/docs/CTCAE v5 Quick Refe rence_5×7(dot)pdf.

TABLE 5
Treatment-Related Adverse Events (TRAE) by Cohort

| | 1 mg/kg | | 3 mg/kg | | 10 mg/kg | | 20 mg/kg | | 30 mg/kg | | Total | |
|---|---|---|---|---|---|---|---|---|---|---|---|---|
| | (N = 4) | | (N = 4) | | (N = 4) | | (N = 4) | | (N = 3) | | (N = 20) | |
| Adverse | GR | | GR | | GR | | GR | | GR | | GR | |
| Event | ANY | GR 3 | ANY | GR 3 | ANY | GR 3 | ANY | GR 3 | ANY | GR 3 | ANY | |
| Anemia | 0 | 0 | 2 | 0 | 2 | 0 | 1 | 0 | 1 | 0 | 6 | (30%) |
| Neutropenia | 0 | 0 | 0 | 0 | 0 | 0 | 0 | 0 | 1 | 0 | 1 | (5%) |
| Lymphocyte | 0 | 0 | 0 | 0 | 1 | 0 | 0 | 0 | 0 | 0 | 1 | (5%) |
| count | | | | | | | | | | | | |
| decreased | | | | | | | | | | | | |
| Plate count | 0 | 0 | 0 | 0 | 1 | 0 | 0 | 0 | 0 | 0 | 1 | (5%) |
| decreased | | | | | | | | | | | | |
| Blood | 0 | 0 | 0 | 0 | 1 | 0 | 0 | 0 | 0 | 0 | 1 | (5%) |
| bilirubin | | | | | | | | | | | | |
| increased | | | | | | | | | | | | |
| Blood LDH | 0 | 0 | 0 | 0 | 0 | 0 | 0 | 0 | 1 | 0 | 1 | (5%) |
| decreased | | | | | | | | | | | | |
| Lipase | 0 | 0 | 0 | 0 | 0 | 0 | 0 | 0 | 1 | 1 | 1 | (5%) |
| increased | | | | | | | | | | | | |
| Fatigue | 0 | 0 | 2 | 0 | 2 | 0 | 1 | 0 | 2 | 0 | 7 | (35%) |
| Chills | 0 | 0 | 1 | 0 | 0 | 0 | 0 | 0 | 0 | 0 | 1 | (5%) |
| Infusion | 0 | 0 | 0 | 0 | 2 | 0 | 2 | 0 | 1 | 0 | 5 | (25%) |
| related | | | | | | | | | | | | |
| reaction | | | | | | | | | | | | |
| Constipation | 0 | 0 | 0 | | 0 | 0 | 1 | 0 | 0 | 0 | 1 | (5%) |
| Diarrhea | 1 | 0 | 1 | 0 | 1 | 0 | 0 | 0 | 0 | 0 | 3 | (15%) |
| Nausea | 0 | 0 | 0 | 0 | 0 | 0 | 1 | 0 | 0 | 0 | 1 | (5%) |
| Dyspnea | 0 | 0 | 0 | 0 | 0 | 0 | 0 | 0 | 1 | 0 | 1 | (5%) |
| Hypotension | 0 | 0 | 0 | 0 | 0 | 0 | 0 | 0 | 1 | 0 | 1 | (5%) |

Effects on Hemoglobin and Reticulocyte Levels

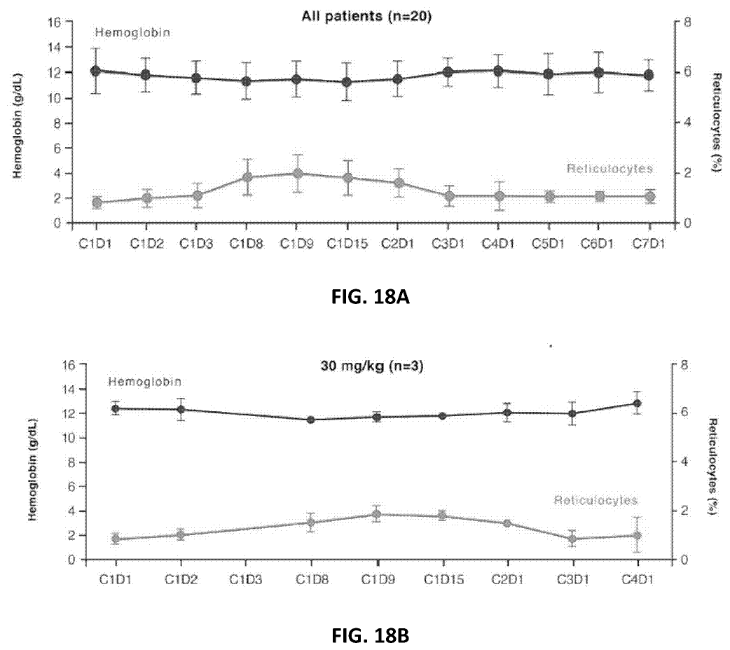

A transient reduction in the hemoglobin levels during the first cycle (i.e., 21 days) was observed across all cohorts. FIG. 18A shows a time course of hemoglobin and reticulocyte levels of all 20 patients, and FIG. 18B shows a time course of hemoglobin and reticulocyte levels in patients receiving the highest dose (30 mg/kg) of lemzoparlimab. The average drop was ˜10% and was not dose dependent. This finding is consistent with the results of pre-clinical Good Laboratory Practice (GLP) toxicity studies. None of the drug-related anemia reported was considered to be severe or hemolytic in nature.

Pharmacokinetics(PK)

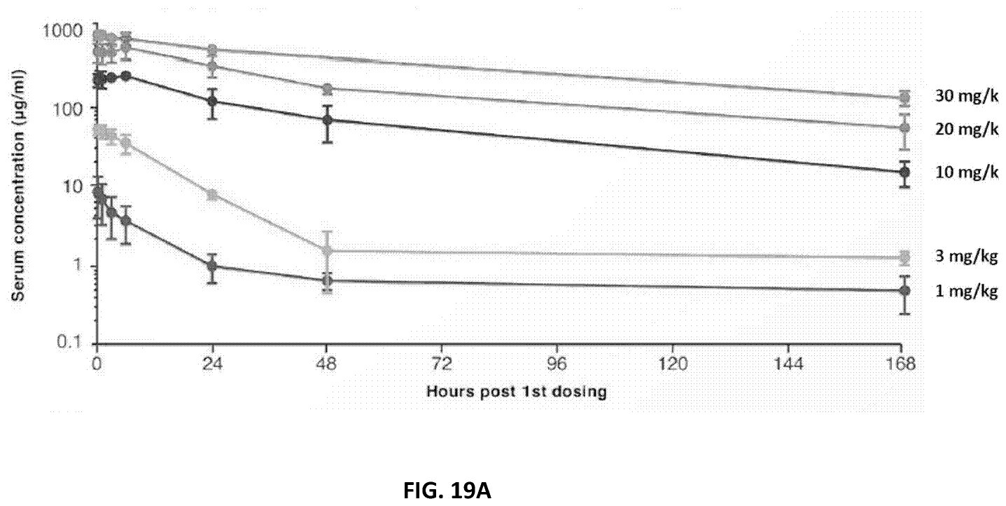

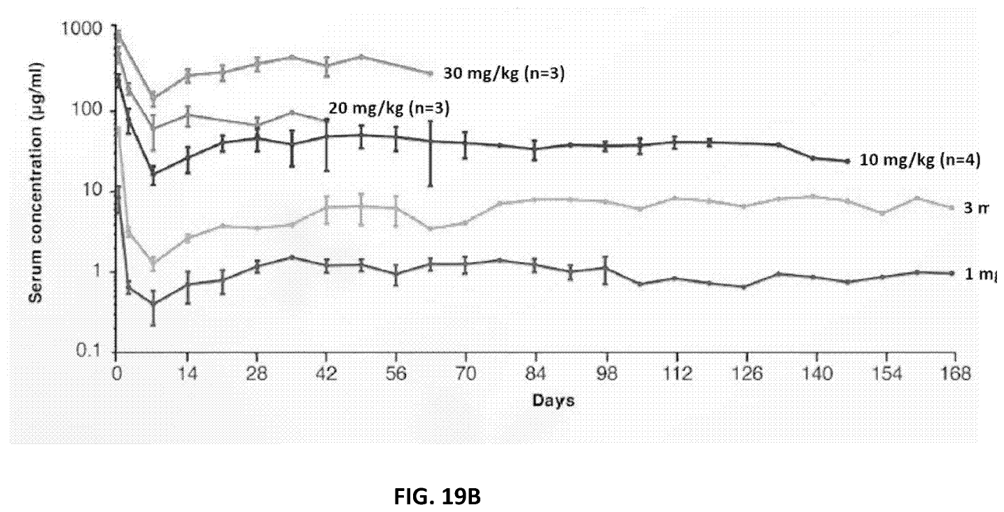

The PK profile of lemzoparlimab appeared linear at the doses higher than 10 mg/kg following a single dose, white its exposure was greater than dose proportional over the dose range of 1 to 10 mg/kg, suggesting that at higher doses, lemzoparlimab can overcome the CD47 sink effect. FIG. 19A shows serum PK of lemzoparlimab in patients following a single dose, and FIG. 19B shows serum PK of lemzoparlimab qw in patients following multiple doses. Five subjects were confirmed positive for anti-drug antibodies (ADA) following the first treatment: 3 were from 1 mg/kg, 1 from 3 mg/kg and 1 from 1 O mg/kg. No impact of ADA was seen on safety or PK.

Pharmacodynamics (PD)

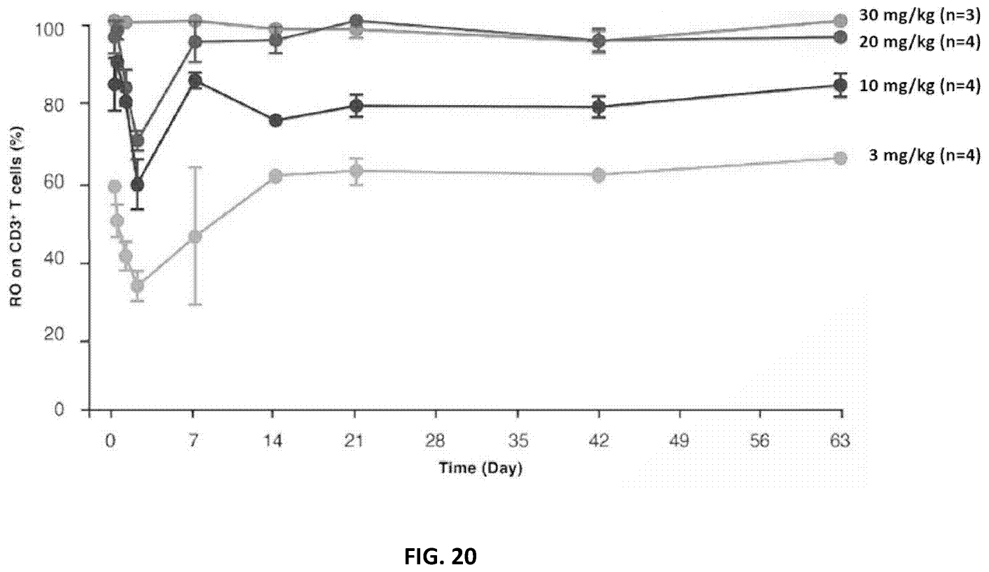

Maximal saturation of CD47 (receptor occupancy RO) on peripheral T cells was achieved at 20 and 30 mg/kg following weekly administration of lemzoparlimab. See FIG. 20.

Preliminary Efficacy

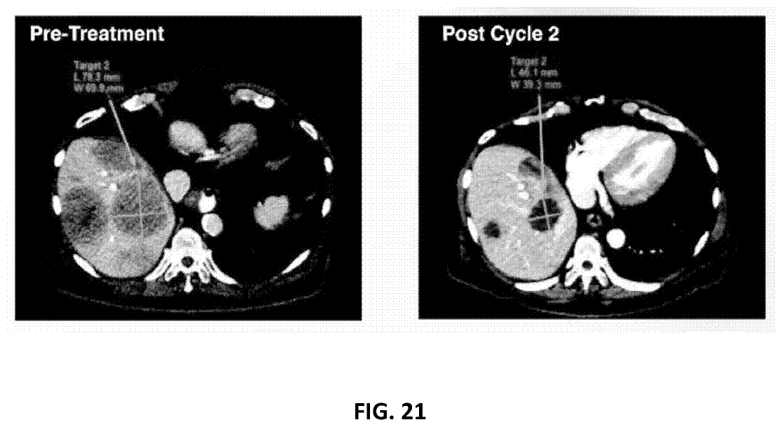

One confirmed Partial Response (PR) was observed (⅓) in the 30 mg/kg monotherapy cohort. 30 mg/kg qw monotherapy ongoing with 5 cycles completed. The patient had metastatic melanoma and had received prior systemic treatment with nivolumab (anti-PD1 antibody) and with ipilimumab (anti-CTLA antibody). See FIG. 21, which shows responding hepatic metastases in the melanoma patient.

Conclusions

Lemzoparlimab appears safe and well-tolerated up to 30 mg/kg on a weekly basis without priming dosing strategy. No dose limiting toxicity was observed and maximum tolerated dose was not reached. The most frequent adverse events included fatigue and transient anemia. No treatment related serious adverse events were noted. Lemzoparlimab PK appears to be linear at mid to high dose levels following a single dose with no significant sink effect. Monotherapy clinical activity (partial response) was observed in one patient (at 30 mg/kg) who had failed prior treatments with checkpoint inhibitors.

### REFERENCES

Willingham et al. (2012) PNAS USA. 109(17): 6662-6667
Liu et al. (2015) PLOS One. 10(9): e0137345
Sikic et al. (2019)J Clin Oncol. 37:946-953.

### Example 22: Initial Clinical Results of Lemzoparlimab, a Differentiated Anti-CD47 Antibody, in Combination with Rituximab in Relapsed and Refractory Non-Hodgkin's Lymphoma

### Introduction

Lemzoparlimab (also known as TJ011133, TJC4, and B2B) is a differentiated CD47 IgG4 antibody targeting a distinct CD47 epitope that confers a unique red blood cell sparing property, while retaining strong anti-tumor activity as demonstrated in patients with solid tumors. (See Example 21.) Lemzoparlimab does not induce significant hematologic toxicity and can be administered without the need of priming dose(s) (e.g., low weekly dose(s) of ˜1 mg/kg) required for other CD47 antibodies. Lemzoparlimab exhibits an enhanced treatment effect when combined with rituximab in lymphoma animal models.

### Methods

This is a Phase 1b study that enrolled relapsed and refractory (R/R) patients with CD20 positive Non-Hodgkin's Lymphoma (NHL) who had at least two prior lines of therapy. The patients were administered with lemzoparlimab in a 3+3 dose escalation design followed by a dose expansion. Lemzoparlimab was administered intravenously at doses of 20 mg/kg weekly or 30 mg/kg weekly in combination with rituximab (375 mg/m² weekly for 5 doses followed by once monthly (q4w or every 28 days) for 3 doses. Safety, tolerability, pharmacokinetics (PK), pharmacodynamics (PD) and anti-tumor activity based on Lugano criteria (see Cheson et al. (2014) Journal of Clinical Oncology. 32:27, 3059-3067 and Van Heertum et al. (2017) Drug Des Devel Ther. 11: 1719-1728) were assessed.

### Results

Eight heavily pre-treated patients with relapsed/refractory non-Hodgkin Lymphoma (R/R NHL) who had progressed on prior CD20 targeted therapies were enrolled to the dose cohorts of 20 mg/kg (n=6) and 30 mg/kg (n=2) of lemzoparlimab in combination with rituximab. The diagnoses included diffuse large B-cell lymphoma (DLBCL) [n=2], mantle cell lymphoma (MCL) [n=1], and follicular lymphoma (FL) [n=5]. Patients had a median age of 63 years (range: 43-83) and a median of 4 prior therapies (range: 2-10). Safety and tolerability: The most common treatment-related adverse events (TRAEs) were infusion-related reactions (n=4), pruritus (n=3), fatigue (n=3), rash (n=2), constipation (n=2), and dyspnea (n=2). All TRAEs were Grade 1 or 2, with one exception who reported Grade 3 TRAEs including pleural effusion, tachycardia, cough, pruritis, fatigue, rash and dyspnea, at 20 mg/kg dose level. Mild hematologic adverse events (AEs) were observed as one isolated episode of anemia and thrombocytopenia, respectively, and no treatment was required. PK and PD: Co-administration of rituximab did not affect the PK or immunogenicity of lemzoparlimab. On average, 80% and 90% CD47 receptor occupancy was detected in biopsied lymph nodes from the patients dosed at 20 and 30 mg/kg, respectively, indicating significant tumor target engagement. Anti-tumor activity: Among 7 efficacy-evaluable patients, 3 complete responses (CR) [1 transformed FL-DLBCL+2 FL] and 1 partial response (PR) of FL were observed (ORR=57%), together with 3 stable disease (SD duration between 3-6 months). The overall disease control rate (DCR) was 100%. Tumor shrinkage was observed in all evaluable patients. One patient was not evaluable for treatment efficacy due to clinical disease progression after withdrawal from the study at the first cycle. The median time to an initial response to the treatment was 2 months and all responders remained in clinical response at time of data cutoff. During continued treatment, two patients developed improved responses. One patient with transformed FL-DLBCL improved from PR at 2^{nd} month to CR at 8^{th} month and another patient with FL improved from SD at 2^{nd} month to PR at 4^{th} month.

### CONCLUSION

Consistent with the monotherapy results (see Example 21), lemzoparlimab given at 20-30 mg/kg in combination with ritthximab is safe and well-tolerated in patients with R/R NHL, without the need for a priming dose commonly used with other therapeutic anti-CD47 antibodies. A high level of intra-tumoral target engagement was reached at both dose levels. The combination therapy exhibited evidence of clinical activity in heavily pre-treated R/R NHL patients who had progressed on prior CD20 targeted therapies.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

AMINO ACID AND NUCLEIC ACID SEQUENCES

| SEQ | | |
|---|---|---|
| ID NO. | Sequence | Description |
| 1 | EVQLVESGGGLVKPGGSLRLSCAASGLTFERAWMNWVRQAPGK | VH of B2B |
| | GLEWVGRIKRKTDGETTDYAAPVKGRFSISRDDSKNTLYLQMNSL | |
| | KTEDTAVYYCAGSNRAFDIWGQGTMVTVSS | |
| 2 | DIVMTQSPDSLAVSLGERATINCKSSQSVLYAGNNRNYLAWYQQ | VL of B2B |
| | KPGQPPKLLINQASTRASGVPDRFSGSGSGTEFTLIISSLQAEDVAIY | |
| | YCQQYYTPPLAFGGGTKLEIK | |
| 3 | EVQLVESGGGLVKPGGSLRLSCAASGLTFERAWMNWVRQAPGK | HC of B2B |
| | GLEWVGRIKRKTDGETTDYAAPVKGRFSISRDDSKNTLYLQMNSL | |
| | KTEDTAVYYCAGSNRAFDIWGQGTMVTVSSASTKGPSVFPLAPCS | |
| | RSTSESTAALGCLVKDYFPEPVTVSWNSGALTSGVHTFPAVLQSSG | |
| | LYSLSSVVTVPSSSLGTKTYTCNVDHKPSNTKVDKRVESKYGPPCP | |
| | PCPAPEFLGGPSVFLFPPKPKDTLMISRTPEVTCVVVDVSQEDPEVQ | |
| | FNWYVDGVEVHNAKTKPREEQFNSTYRVVSVLTVLHQDWLNGK | |
| | EYKCKVSNKGLPSSIEKTISKAKGQPREPQVYTLPPSQEEMTKNQV | |
| | SLTCLVKGFYPSDIAVEWESNGQPENNYKTTPPVLDSDGSFFLYSR | |
| | LTVDKSRWQEGNVFSCSVMHEALHNHYTQKSLSLSLGK | |
| 4 | DIVMTQSPDSLAVSLGERATINCKSSQSVLYAGNNRNYLAWYQQ | LC of B2B |
| | KPGQPPKLLINQASTRASGVPDRFSGSGSGTEFTLIISSLQAEDVAIY | |
| | YCQQYYTPPLAFGGGTKLEIKRTVAAPSVFIFPPSDEQLKSGTASV | |
| | VCLLNNFYPREAKVQWKVDNALQSGNSQESVTEQDSKDSTYSLSS | |
| | TLTLSKADYEKHKVYACEVTHQGLSSPVTKSFNRGEC | |
| 5 | RAWMN | HCDR1 of B2B |
| | | (Kabat) |
| 6 | RIKRKTDGETTDY AAPVKG | HCDR2 of B2B |
| | | (Kabat) |
| 7 | SNRAFDI | HCDR3 of B2B |
| | | (Kabat) |
| 8 | KSSQSVLYAGNNRNYLA | LCDR1 of B2B |
| | | (Kabat) |
| 9 | QASTRAS | LCDR2 of B2B |
| | | (Kabat) |
| 10 | QQYYTPPLA | LCDR3 of B2B |
| | | (Kabat) |
| 11 | KVQLVESGGGLVKPGGSLRLSCAASGLTFERAWMNWVRQAPGK | VH of C3C |
| | GLEWVGRIKRKTDGETTDYAAPVKGRFSISRDDSKNTLYLQMNSL | |
| | KTEDTAVYYCAGSNRAFDIWGQGTMVTVSA | |
| 12 | DIVMTQSPDSLAVSLGERATINCKSSQSVLYAGNNRNYLAWYQQ | VL of C3C |
| | KPGQPPKLLINQASTRASGVPDRFSGSGSGTEFTLIISSLQAEDVAIY | |
| | YCQQYYTPPLAFGGGTKLEIK | |
| 13 | KVQLVESGGGLVKPGGSLRLSCAASGLTFERAWMNWVRQAPGK | HC of C3C |
| | GLEWVGRIKRKTDGETTDYAAPVKGRFSISRDDSKNTLYLQMNSL | |
| | KTEDTAVYYCAGSNRAFDIWGQGTMVTVSAASTKGPSVFPLAPCS | |
| | RSTSESTAALGCLVKDYFPEPVTVSWNSGALTSGVHTFPAVLQSSG | |
| | LYSLSSVVTVPSSSLGTKTYTCNVDHKPSNTKVDKRVESKYGPPCP | |
| | PCPAPEFLGGPSVFLFPPKPKDTLMISRTPEVTCVVVDVSQEDPEVQ | |
| | FNWYVDGVEVHNAKTKPREEQFNSTYRVVSVLTVLHQDWLNGK | |
| | EYKCKVSNKGLPSSIEKTISKAKGQPREPQVYTLPPSQEEMTKNQV | |
| | SLTCLVKGFYPSDIAVEWESNGQPENNYKTTPPVLDSDGSFFLYSR | |
| | LTVDKSRWQEGNVFSCSVMHEALHNHYTQKSLSLSLGK | |
| 14 | DIVMTQSPDSLAVSLGERATINCKSSQSVLYAGNNRNYLAWYQQ | LC of C3C |
| | KPGQPPKLLINQASTRASGVPDRFSGSGSGTEFTLIISSLQAEDVAIY | |
| | YCQQYYTPPLAFGGGTKLEIKRTVAAPSVFIFPPSDEQLKSGTASV | |
| | VCLLNNFYPREAKVQWKVDNALQSGNSQESVTEQDSKDSTYSLSS | |
| | TLTLSKADYEKHKVYACEVTHQGLSSPVTKSFNRGEC | |
| 15 | RAWMN | CDR-H1 of C3C |
| | | (Kabat) |
| 16 | RIKRKTDGETTDYAAPVKG | CDR-H2 of C3C |
| | | (Kabat) |
| 17 | SNRAFDI | CDR-H3 of C3C |
| | | (Kabat) |
| 18 | KSSQSVLYAGNNRNYLA | CDR-LI of C3C |
| | | (Kabat) |
| 19 | QASTRAS | CDR-L2 of C3C |
| | | (Kabat) |
| 20 | QQYYTPPLA | CDR-L3 of C3C |
| | | (Kabat) |
| 21 | GLTFERA | CDR-H1of B2B |
| | | (Chothia) |
| 22 | KRKTDGET | CDR-H2 of B2B |
| | | (Chothia) |
| 23 | SNRAFDI | CDR-H3 of B2B |
| | | (Chothia) |
| 24 | KSSQSVLYAGNNRNYLA | CDR-L1 of B2B |
| | | (Chothia) |
| 25 | QASTRAS | CDR-L2 of B2B |
| | | (Chothia) |
| 26 | QQYYTPPLA | CDR-L3 of B2B |
| | | (Chothia) |
| 27 | GLTFERAW | CDR-H1 of B2B |
| | | (IMGT) |
| 28 | IKRKTDGETT | CDR-H2 of B2B |
| | | (IMGT) |
| 29 | AGSNRAFDI | CDR-H3 of B2B |
| | | (IMGT) |
| 30 | QSVLYAGNNRNY | CDR-L1 of B2B |
| | | (IMGT) |
| 31 | QA | CDR-L2 of B2B |
| | | (IMGT) |
| 32 | QQYYTPPLA | CDR-L3 of B2B |
| | | (IMGT) |
| 33 | GLTFERAWMN | CDR-H1 of B2B |
| | | (AbM) |
| 34 | RIKRKTDGETTD | CDR-H2 of B2B |
| | | (AbM) |
| 35 | SNRAFDI | CDR-H3 of B2B |
| | | (AbM) |
| 36 | KSSQSVLYAGNNRNYLA | CDR-L1 of B2B |
| | | (AbM) |
| 37 | QASTRAS | CDR-L2 of B2B |
| | | (AbM) |
| 38 | QQYYTPPLA | CDR-L3 of B2B |
| | | (AbM) |
| 39 | ERAWMN | CDR-H1 of B2B |
| | | (Contact) |
| 40 | WVGRIKRKTDGETTD | CDR-H2 of B2B |
| | | (Contact) |
| 41 | AGSNRAFD | CDR-H3 of B2B |
| | | (Contact) |
| 42 | LYAGNNRNYLAWY | CDR-L1 of B2B |
| | | (Contact) |
| 43 | LLINQASTRA | CDR-L2 of B2B |
| | | (Contact) |
| 44 | QQYYTPPL | CDR-L3 of B2B |
| | | (Contact) |
| 45 | GAGGTGCAGCTGGTGGAGAGCGGAGGCGGACTCGTGAAGCCTG | VH of B2B |
| | GAGGAAGCCTGAGGCTGTCCTGTGCCGCTTCCGGCCTCACCTTC | |
| | GAGCGGGCTTGGATGAACTGGGTGAGGCAGGCCCCTGGAAAGG | |
| | GCCTGGAATGGGTGGGCCGGATCAAGAGGAAAACAGATGGCG | |
| | AGACCACCGATTACGCCGCTCCCGTGAAGGGCCGGTTTAGCAT | |
| | CTCCAGGGACGACTCCAAGAACACCCTGTATCTGCAGATGAAC | |
| | AGCCTGAAGACCGAGGACACCGCTGTGTACTACTGCGCTGGCA | |
| | GCAACAGGGCCTTTGATATCTGGGGCCAGGGCACCATGGTGAC | |
| | AGTGTCCTCC | |
| 46 | GACATCGTGATGACCCAGTCCCCTGATTCCCTGGCCGTGAGCCT | VL of B2B |
| | GGGCGAAAGGGCTACCATCAACTGCAAGTCCTCCCAGAGCGTG | |
| | CTGTACGCCGGCAACAACCGGAACTATCTGGCTTGGTACCAGC | |
| | AGAAGCCCGGCCAGCCTCCCAAGCTGCTGATCAACCAGGCTAG | |
| | CACCAGGGCTTCCGGCGTGCCTGATAGGTTCAGCGGCTCCGGCT | |
| | CCGGCACCGAGTTTACCCTGATCATCTCCTCCCTGCAGGCCGAG | |
| | GATGTGGCCATCTACTACTGCCAGCAGTACTACACCCCTCCTCT | |
| | GGCCTTTGGCGGCGGCACCAAGCTGGAGATCAAG | |
| 47 | GAGGTGCAGCTGGTGGAGAGCGGAGGCGGACTCGTGAAGCCTG | HC of B2B |
| | GAGGAAGCCTGAGGCTGTCCTGTGCCGCTTCCGGCCTCACCTTC | |
| | GAGCGGGCTTGGATGAACTGGGTGAGGCAGGCCCCTGGAAAGG | |
| | GCCTGGAATGGGTGGGCCGGATCAAGAGGAAAACAGATGGCG | |
| | AGACCACCGATTACGCCGCTCCCGTGAAGGGCCGGTTTAGCAT | |
| | CTCCAGGGACGACTCCAAGAACACCCTGTATCTGCAGATGAAC | |
| | AGCCTGAAGACCGAGGACACCGCTGTGTACTACTGCGCTGGCA | |
| | GCAACAGGGCCTTTGATATCTGGGGCCAGGGCACCATGGTGAC | |
| | AGTGTCCTCCGCCTCCACAAAGGGACCTTCCGTGTTCCCTCTGG | |
| | CCCCTTGTTCCCGGTCCACCTCCGAAAGCACCGCTGCTCTGGGC | |
| | TGCCTCGTCAAGGACTACTTCCCTGAGCCCGTGACCGTGAGCTG | |
| | GAACTCCGGCGCTCTGACAAGCGGCGTGCATACCTTCCCTGCCG | |
| | TGCTGCAAAGCAGCGGCCTGTATAGCCTGAGCAGCGTGGTGAC | |
| | CGTGCCTAGCTCCTCCCTGGGCACCAAAACCTACACCTGCAATG | |
| | TGGACCACAAGCCTTCCAACACCAAGGTGGACAAGCGGGTCGA | |
| | GTCCAAGTACGGCCCTCCTTGCCCTCCCTGCCCCGCTCCCGAGT | |
| | TTCTGGGAGGACCCAGCGTGTTCCTCTTCCCCCCTAAGCCCAAG | |
| | GACACCCTGATGATCAGCCGGACACCTGAGGTCACCTGCGTGG | |
| | TGGTGGATGTGAGCCAAGAGGATCCTGAGGTCCAGTTCAACTG | |
| | GTACGTGGACGGAGTGGAGGTGCATAACGCCAAGACCAAGCCT | |
| | CGGGAGGAGCAGTTCAACTCCACCTATAGGGTGGTGAGCGTGC | |
| | TCACAGTGCTCCACCAGGACTGGCTGAACGGCAAGGAGTACAA | |
| | ATGCAAGGTGTCCAACAAGGGACTCCCCAGCAGCATCGAAAAG | |
| | ACCATCAGCAAGGCCAAAGGCCAGCCCAGGGAACCCCAGGTGT | |
| | ACACACTGCCCCCCTCCCAAGAGGAAATGACCAAGAATCAGGT | |
| | GTCCCTGACCTGCCTGGTGAAAGGCTTTTACCCCAGCGACATCG | |
| | CTGTCGAGTGGGAGAGCAACGGCCAGCCTGAGAATAACTATAA | |
| | GACCACCCCCCCCGTGCTGGATAGCGACGGATCCTTCTTCCTCT | |
| | ACTCCCGGCTGACCGTGGATAAGTCCCGGTGGCAGGAGGGCAA | |
| | CGTGTTCAGCTGCTCCGTCATGCACGAGGCCCTGCATAACCACT | |
| | ACACCCAGAAGTCCCTGAGCCTGTCCCTGGGCAAGTGA | |
| 48 | GACATCGTGATGACCCAGTCCCCTGATTCCCTGGCCGTGAGCCT | LC of B2B |
| | GGGCGAAAGGGCTACCATCAACTGCAAGTCCTCCCAGAGCGTG | |
| | CTGTACGCCGGCAACAACCGGAACTATCTGGCTTGGTACCAGC | |
| | AGAAGCCCGGCCAGCCTCCCAAGCTGCTGATCAACCAGGCTAG | |
| | CACCAGGGCTTCCGGCGTGCCTGATAGGTTCAGCGGCTCCGGCT | |
| | CCGGCACCGAGTTTACCCTGATCATCTCCTCCCTGCAGGCCGAG | |
| | GATGTGGCCATCTACTACTGCCAGCAGTACTACACCCCTCCTCT | |
| | GGCCTTTGGCGGCGGCACCAAGCTGGAGATCAAGAGGACAGTG | |
| | GCCGCCCCCTCCGTGTTCATTTTCCCTCCCTCCGACGAGCAGCT | |
| | GAAGTCCGGCACCGCCTCCGTGGTGTGCCTGCTGAACAACTTCT | |
| | ACCCCAGGGAGGCCAAGGTGCAGTGGAAGGTGGACAATGCCCT | |
| | GCAGAGCGGCAACAGCCAGGAGAGCGTCACCGAGCAGGACTC | |
| | CAAAGACAGCACATACAGCCTGTCCAGCACCCTGACCCTGTCC | |
| | AAGGCTGACTATGAGAAGCACAAGGTGTACGCCTGCGAGGTGA | |
| | CCCACCAGGGACTGAGCTCCCCTGTGACCAAGTCCTTCAACCG | |
| | GGGAGAGTGCTGA | |
| 49 | CGGGCTTGGATGAAC | HCDR1 of B2B |
| | | (Kabat) |
| 50 | CGGATCAAGAGGAAAACAGATGGCGAGACCACCGATTACGCC | HCDR2 of B2B |
| | GCTCCCGTGAAGGGC | (Kabat) |
| 51 | AGCAACAGGGCCTTTGATATC | HCDR3 of B2B |
| | | (Kabat) |
| 52 | AAGTCCTCCCAGAGCGTGCTGTACGCCGGCAACAACCGGAACT | LCDR1 of B2B |
| | ATCTGGCT | (Kabat) |
| 53 | CAGGCTAGCACCAGGGCTTCC | LCDR2 of B2B |
| | | (Kabat) |
| 54 | CAGCAGTACTACACCCCTCCTCTGGCC | LCDR3 of B2B |
| | | (Kabat) |
| 55 | EVQLVESGGGLVKPGGSLRLSCAASGLTFERAWMNWVRQAPGK | HC of B2B |
| | GLEWVGRIKRKTDGETTDYAAPVKGRFSISRDDSKNTLYLQMNSL | |
| | KTEDTAVYYCAGSNRAFDIWGQGTMVTVSSASTKGPSVFPLAPCS | |
| | RSTSESTAALGCLVKDYFPEPVTVSWNSGALTSGVHTFPAVLQSSG | |
| | LYSLSSVVTVPSSSLGTKTYTCNVDHKPSNTKVDKRVESKYGPPCP | |
| | PCPAPEFLGGPSVFLFPPKPKDTLMISRTPEVTCVVVDVSQEDPEVQ | |
| | FNWYVDGVEVHNAKTKPREEQFNSTYRVVSVLTVLHQDWLNGK | |
| | EYKCKVSNKGLPSSIEKTISKAKGQPREPQVYTLPPSQEEMTKNQV | |
| | SLTCLVKGFYPSDIAVEWESNGQPENNYKTTPPVLDSDGSFFLYSR | |
| | LTVDKSRWQEGNVFSCSVMHEALHNHYTQKSLSLSLG | |

1. An antibody or immunologically active fragment thereof that specifically binds to human CD47 (hCD47), comprising:
(a) a heavy chain variable (V_{H}) domain that comprises (1) a glutamic acid residue (E) at its N-terminus; (2) a CDR-H1 comprising RAWMN (SEQ ID NO: 5); (3) a CDR-H2 comprising RIKRKTDGETTDYAAPVKG (SEQ ID NO: 6); (4) a CDR-H3 comprising SNRAFDI (SEQ ID NO: 7); and (5) a serine (S) at its C-terminus; and
(b) a light chain variable (V_{L}) domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10).

2. The anti-CD47 antibody or immunologically active fragment thereof of claim 1, wherein the N-terminal amino acid of the V_{H} domain corresponds to position H1 according to the Kabat numbering system, and the C-terminal amino acid of the V_{H} domain corresponds to position H113 according to the Kabat numbering system.

3. The anti-CD47 antibody or immunologically active fragment thereof of claim 1, wherein the N-terminal amino acid of the V_{H} domain corresponds to position H1 according to the Chothia numbering system, and the C-terminal amino acid of the V_{H} domain corresponds to position H113 according to the Chothia numbering system.

4. The anti-CD47 antibody or immunologically active fragment thereof of claim 1, wherein the N-terminal amino acid of the V_{H} domain corresponds to position H1 according to the IMGT numbering system, and the C-terminal amino acid of the V_{H} domain corresponds to position H128 according to the IMGT numbering system.

5. The anti-CD47 antibody or immunologically active fragment thereof of any one of claims 1-4, wherein the N-terminal amino acid of the V_{H} domain corresponds to amino acid 1 of SEQ ID NO: 1, and the C-terminal amino acid of the V_{H} domain corresponds to amino acid 118 of SEQ ID NO: 1.

6. The anti-CD47 antibody or immunologically active fragment thereof of any one of claims 1-5, wherein the V_{H} comprises an amino acid sequence that has at least 95% identity to SEQ ID NO: 1, and the V_{L} comprises an amino acid sequence that has at least 95% identity to SEQ ID NO: 2.

7. The anti-CD47 antibody or immunologically active fragment thereof of any one of claims 1-6, wherein the V_{H} comprises SEQ ID NO: 1, and the V_{L} comprises SEQ ID NO: 2.

8. The anti-CD47 antibody or immunologically active fragment thereof of any one of claims 1-7, comprising an Fc domain.

9. The anti-CD47 antibody or immunologically active fragment thereof of claim 8, comprising a human IgG Fc domain.

10. The anti-CD47 antibody or immunologically active fragment thereof of claim 9, wherein the human IgG Fc domain is an IgG1, IgG2, IgG3, or IgG4 Fc domain.

11. The anti-CD47 antibody of any one of claims 1-10, wherein the antibody is a full length antibody.

12. The anti-CD47 antibody of any one of claims 1-11, comprising a heavy chain that comprises SEQ ID NO: 3 or SEQ ID NO: 55 and a light chain that comprises SEQ ID NO: 4.

13. The immunologically active fragment of the anti-CD47 antibody of any one of claims 1-10, wherein the fragment is a Fab, a Fab′, a F(ab)′2, a Fab′-SH, a single-chain Fv (scFv), an Fv fragment, or a linear antibody.

14. The anti-CD47 antibody or immunologically active fragment thereof of any one of claims 1-13, wherein the antibody or fragment thereof is a monoclonal antibody or fragment thereof.

15. The anti-CD47 antibody or immunologically active fragment thereof of any one of claims 1-14, wherein the antibody or fragment thereof is chimeric or humanized.

16. The anti-CD47 antibody or immunologically active fragment thereof of any one of claims 1-15, wherein the antibody or fragment binds to hCD47 expressed on the surface of a cancer cell.

17. The anti-CD47 antibody or immunologically active fragment thereof of claim 16, wherein the cancer cell is a SK-OV-3 cell, a Toledo cell, a K562 cell, a HCC827 cell, a Jurkat cell, a U937 cell, a TF-1 cell, a Raji cell, a SU-DHL-4 cell, a MDA-MB-231 cell, an A375 cell, or a SK-MES-1 cell.

18. The anti-CD47 antibody or immunologically active fragment thereof of claim 16, wherein the cancer cell is a solid tumor cancer.

19. The anti-CD47 antibody or immunologically active fragment thereof of claim 18, wherein the solid tumor cancer is lung cancer, ovarian cancer, colorectal cancer, pancreatic cancer, sarcoma cancer, head and neck cancer, gastric cancer, renal cancer, or skin cancer.

20. The anti-CD47 antibody or immunologically active fragment thereof of claim 16, wherein the cancer cell is a hematological cancer.

21. The anti-CD47 antibody or immunologically active fragment thereof of claim 20, wherein the hematological cancer is non-Hodgkin lymphoma.

22. The anti-CD47 antibody or immunologically active fragment thereof of any one of claims 1-21, wherein the antibody or fragment does not bind to hCD47 expressed on the surface of a blood cell.

23. The anti-CD47 antibody or immunologically active fragment thereof of claim 22, wherein the blood cell is an erythrocyte.

24. The anti-CD47 antibody or immunologically active fragment thereof of any one of claims 1-23, wherein the binding of the antibody or fragment thereof to hCD47 prevents interaction of the hCD47 with signal-regulatory-protein α (SIRPα).

25. The anti-CD47 antibody or immunologically active fragment thereof of claim 24, wherein the SIRPα is human SIRPα (hSIRPα).

26. The anti-CD47 antibody or immunologically active fragment thereof of any one of claims 1-25, wherein the binding of the antibody or fragment thereof to hCD47 expressed on the surface of a cancer cell promotes macrophage-mediated phagocytosis of the cancer cell.

27. The anti-CD47 antibody or immunologically active fragment thereof of claim 26, wherein the cancer cell is a SK-OV-3 cell, a Toledo cell, a K562 cell, a HCC827 cell, a Jurkat cell, a U937 cell, a TF-1 cell, a Raji cell, a SU-DHL-4 cell, a MDA-MB-231 cell, an A375 cell, or a SK-MES-1 cell.

28. The anti-CD47 antibody or immunologically active fragment thereof of claim 26, wherein the cancer cell is a solid tumor cancer.

29. The anti-CD47 antibody or immunologically active fragment thereof of claim 28, wherein the solid tumor cancer is lung cancer, ovarian cancer, colorectal cancer, pancreatic cancer, sarcoma cancer, head and neck cancer, gastric cancer, renal cancer, or skin cancer.

30. The anti-CD47 antibody or immunologically active fragment thereof of claim 26, wherein the cancer cell is a hematological cancer.

31. The anti-CD47 antibody or immunologically active fragment thereof of claim 30, wherein the hematological cancer is non-Hodgkin lymphoma.

32. The anti-CD47 antibody or immunologically active fragment thereof of any one of claims 1-31, wherein administration of the antibody or fragment thereof to a subject does not cause a significant level of hemagglutination in the subject or depletion of the subject's red blood cells.

33. A nucleic acid encoding the anti-CD47 antibody or immunologically active fragment thereof any one of claims 1-32.

34. A vector comprising the nucleic acid of claim 33.

35. A host cell comprising the nucleic acid of claim 33, or the vector of claim 34.

36. The host cell of claim 35, wherein the host cell is a mammalian cell.

37. The host cell of claim 36, wherein the mammalian cell is a Chinese hamster ovary (CHO) cell.

38. The host cell of claim 37, wherein the CHO cell is a CHO-K1 cell.

39. A method of producing an anti-CD47 antibody or immunologically active fragment thereof, comprising:
a) culturing the host cell of any one of claims 35-38 under conditions effective to cause expression of the anti-CD47 antibody or antigen-binding fragment thereof; and
b) recovering the anti-CD47 antibody or immunologically active fragment thereof expressed by the host cell.

40. A pharmaceutical composition comprising the anti-CD47 antibody or immunologically active fragment thereof of any one of claims 1-32 and pharmaceutically acceptable carrier.

41. A method of treating cancer in a subject, comprising administering an effective amount of an anti-CD47 antibody to the subject,
wherein the anti-CD47 antibody comprises
(a) a heavy chain variable domain (V_{H}) that comprises (1) a CDR-H1 comprising RAWMN (SEQ ID NO: 5); (2) a CDR-H2 comprising RIKRKTDGETTDYAAPVKG (SEQ ID NO: 6); and (3) a CDR-H3 comprising SNRAFDI (SEQ ID NO: 7);
(b) a light chain variable (V_{L}) domain that comprises (1) a CDR-L1 comprising KSSQSVLYAGNNRNYLA (SEQ ID NO: 8); (2) a CDR-L2 comprising QASTRAS (SEQ ID NO: 9); and (3) a CDR-L3 comprising QQYYTPPLA (SEQ ID NO: 10).

42. The method of claim 41, wherein the cancer is solid tumor.

43. The method of claim 42 or 43, wherein the solid tumor is a lung tumor, an ovarian tumor, a colorectal tumor, a pancreatic tumor, a sarcoma tumor, a head and neck tumor, a gastric tumor, a renal tumor, or a skin tumor.

44. The method of claim 42 or 43, wherein the solid tumor is relapsed and/or refractory solid tumor.

45. The method of claim 41, wherein the cancer is non-Hodgkin lymphoma (NHL), and wherein the method further comprises administering an effective amount of rituximab to the subject.

46. The method of claim 45, wherein the NHL is follicular lymphoma (FL), diffuse large B-cell lymphoma (DLBCL), or mantle cell lymphoma (MCL).

47. The method of claim 45 or 46, wherein the NHL is relapsed/refractory NHL.

48. The method of any one of claims 45-47, wherein the subject has undergone at least one prior treatment for NHL.

49. The method of claim 48, wherein the subject has undergone between 2 and 10 prior therapies for NHL.

50. The method of claim 48 or 49, wherein the subject has undergone prior treatment for NHL with an agent that targets CD20.

51. The method of claim 50, wherein the subject progressed during or after the prior therapy with an agent that targets CD20.

52. The method of any one of claims 41-51, wherein the anti-CD47 antibody comprises a human IgG4 constant region or a variant thereof comprising an S233P mutation (wherein numbering is according to the EU index).

53. The method of any one of claims 41-52, wherein the anti-CD47 antibody is administered to the subject at a dose of 10 mg/kg.

54. The method of any one of claims 41-52, wherein the anti-CD47 antibody is administered to the subject at a dose of 20 mg/kg.

55. The method of any one of claims 41-52, wherein the anti-CD47 antibody is administered to the subject at a dose of 30 mg/kg.

56. The method of any one of claims 41-55, wherein the anti-CD47 antibody is administered to the subject once every week (qw).

57. The method of any one of claims 41-56, wherein the anti-CD47 antibody is administered to the subject via intravenous (IV) in fusion.

58. The method of any one of claims 45-57, wherein the rituximab is administered at a dose of 375 mg/m² once a week (qw) for a first five weeks and at a dose of 375 mg/m² once every 4 weeks (q4w) following the first five weeks.

59. The method of any one of claims 41-58, wherein the subject does not experience significant hematological toxicity due to the treatment with the anti-CD47 antibody.

60. The method of claim 59, wherein the subject does not experience any hematological toxicity due to treatment with the anti-CD47 antibody.

61. The method of claim 59 or 60, wherein the hematological toxicity comprises anemia, cytopenia, and/or hemagglutination.

62. The method of any one of claims 41-61, wherein the V_{H} of the anti-CD47 antibody comprises SEQ ID NO: 1, and the V_{L} of the anti-CD47 antibody comprises SEQ ID NO: 2.

63. The method of an one of claims 41-62, wherein the heavy chain of the anti-CD47 antibody comprises SEQ ID NO: 3 or SEQ ID NO: 55 and the light chain of the anti-CD47 antibody comprises SEQ ID NO: 4.

64. A kit for treating cancer comprising the antibody of any one of claims 1-32 or the composition of claim 40.


SEQUENCE LISTING (55 sequences)

SEQ ID NO: 1 - protein, 118 aa, Artificial Sequence
  Synthetic Construct
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSS

SEQ ID NO: 2 - protein, 113 aa, Artificial Sequence
  Synthetic Construct
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIK

SEQ ID NO: 3 - protein, 445 aa, Artificial Sequence
  Synthetic Construct
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - protein, 220 aa, Artificial Sequence
  Synthetic Construct
DIVMTQSPDSLAVSLGERATINCKSSQSVLYAGNNRNYLAWYQQKPGQPPKLLINQASTRASGVPDRFSGSGSGTEFTLIISSLQAEDVAIYYCQQYYTPPLAFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 5 aa, Artificial Sequence
  Synthetic Construct
RAWMN

SEQ ID NO: 6 - protein, 19 aa, Artificial Sequence
  Synthetic Construct
RIKRKTDGETTDYAAPVKG

SEQ ID NO: 7 - protein, 7 aa, Artificial Sequence
  Synthetic Construct
SNRAFDI

SEQ ID NO: 8 - protein, 17 aa, Artificial Sequence
  Synthetic Construct
KSSQSVLYAGNNRNYLA

SEQ ID NO: 9 - protein, 7 aa, Artificial Sequence
  Synthetic Construct
QASTRAS

SEQ ID NO: 10 - protein, 9 aa, Artificial Sequence
  Synthetic Construct
QQYYTPPLA

SEQ ID NO: 11 - protein, 118 aa, Artificial Sequence
  Synthetic Construct
KVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSA

SEQ ID NO: 12 - protein, 113 aa, Artificial Sequence; identical to SEQ ID NO: 2
  Synthetic Construct

SEQ ID NO: 13 - protein, 445 aa, Artificial Sequence
  Synthetic Construct
KVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 220 aa, Artificial Sequence; identical to SEQ ID NO: 4
  Synthetic Construct

SEQ ID NO: 15 - protein, 5 aa, Artificial Sequence
  Synthetic Construct
RAWMN

SEQ ID NO: 16 - protein, 19 aa, Artificial Sequence
  Synthetic Construct
RIKRKTDGETTDYAAPVKG

SEQ ID NO: 17 - protein, 7 aa, Artificial Sequence
  Synthetic Construct
SNRAFDI

SEQ ID NO: 18 - protein, 17 aa, Artificial Sequence
  Synthetic Construct
KSSQSVLYAGNNRNYLA

SEQ ID NO: 19 - protein, 7 aa, Artificial Sequence
  Synthetic Construct
QASTRAS

SEQ ID NO: 20 - protein, 9 aa, Artificial Sequence
  Synthetic Construct
QQYYTPPLA

SEQ ID NO: 21 - protein, 7 aa, Artificial Sequence
  Synthetic Construct
GLTFERA

SEQ ID NO: 22 - protein, 8 aa, Artificial Sequence
  Synthetic Construct
KRKTDGET

SEQ ID NO: 23 - protein, 7 aa, Artificial Sequence
  Synthetic Construct
SNRAFDI

SEQ ID NO: 24 - protein, 17 aa, Artificial Sequence
  Synthetic Construct
KSSQSVLYAGNNRNYLA

SEQ ID NO: 25 - protein, 7 aa, Artificial Sequence
  Synthetic Construct
QASTRAS

SEQ ID NO: 26 - protein, 9 aa, Artificial Sequence
  Synthetic Construct
QQYYTPPLA

SEQ ID NO: 27 - protein, 8 aa, Artificial Sequence
  Synthetic Construct
GLTFERAW

SEQ ID NO: 28 - protein, 10 aa, Artificial Sequence
  Synthetic Construct
IKRKTDGETT

SEQ ID NO: 29 - protein, 9 aa, Artificial Sequence
  Synthetic Construct
AGSNRAFDI

SEQ ID NO: 30 - protein, 12 aa, Artificial Sequence
  Synthetic Construct
QSVLYAGNNRNY

SEQ ID NO: 31 - protein, 2 aa, Artificial Sequence
  Synthetic Construct
QA

SEQ ID NO: 32 - protein, 9 aa, Artificial Sequence
  Synthetic Construct
QQYYTPPLA

SEQ ID NO: 33 - protein, 10 aa, Artificial Sequence
  Synthetic Construct
GLTFERAWMN

SEQ ID NO: 34 - protein, 12 aa, Artificial Sequence
  Synthetic Construct
RIKRKTDGETTD

SEQ ID NO: 35 - protein, 7 aa, Artificial Sequence
  Synthetic Construct
SNRAFDI

SEQ ID NO: 36 - protein, 17 aa, Artificial Sequence
  Synthetic Construct
KSSQSVLYAGNNRNYLA

SEQ ID NO: 37 - protein, 7 aa, Artificial Sequence
  Synthetic Construct
QASTRAS

SEQ ID NO: 38 - protein, 9 aa, Artificial Sequence
  Synthetic Construct
QQYYTPPLA

SEQ ID NO: 39 - protein, 6 aa, Artificial Sequence
  Synthetic Construct
ERAWMN

SEQ ID NO: 40 - protein, 15 aa, Artificial Sequence
  Synthetic Construct
WVGRIKRKTDGETTD

SEQ ID NO: 41 - protein, 8 aa, Artificial Sequence
  Synthetic Construct
AGSNRAFD

SEQ ID NO: 42 - protein, 13 aa, Artificial Sequence
  Synthetic Construct
LYAGNNRNYLAWY

SEQ ID NO: 43 - protein, 10 aa, Artificial Sequence
  Synthetic Construct
LLINQASTRA

SEQ ID NO: 44 - protein, 8 aa, Artificial Sequence
  Synthetic Construct
QQYYTPPL

SEQ ID NO: 45 - DNA, 354 bp, Artificial Sequence
  Synthetic Construct
gaggtgcagctggtggagagcggaggcggactcgtgaagcctggaggaagcctgaggctgtcctgtgccgcttccggcctcaccttcgagcgggcttggatgaactgggtgaggcaggcccctggaaagggcctggaatgggtgggccggatcaagaggaaaacagatggcgagaccaccgattacgccgctcccgtgaagggccggtttagcatctccagggacgactccaagaacaccctgtatctgcagatgaacagcctgaagaccgaggacaccgctgtgtactactgcgctggcagcaacagggcctttgatatctggggccagggcaccatggtgacagtgtcctcc

SEQ ID NO: 46 - DNA, 339 bp, Artificial Sequence
  Synthetic Construct
gacatcgtgatgacccagtcccctgattccctggccgtgagcctgggcgaaagggctaccatcaactgcaagtcctcccagagcgtgctgtacgccggcaacaaccggaactatctggcttggtaccagcagaagcccggccagcctcccaagctgctgatcaaccaggctagcaccagggcttccggcgtgcctgataggttcagcggctccggctccggcaccgagtttaccctgatcatctcctccctgcaggccgaggatgtggccatctactactgccagcagtactacacccctcctctggcctttggcggcggcaccaagctggagatcaag

SEQ ID NO: 47 - DNA, 1338 bp, Artificial Sequence
  Synthetic Construct
gaggtgcagctggtggagagcggaggcggactcgtgaagcctggaggaagcctgaggctgtcctgtgccgcttccggcctcaccttcgagcgggcttggatgaactgggtgaggcaggcccctggaaagggcctggaatgggtgggccggatcaagaggaaaacagatggcgagaccaccgattacgccgctcccgtgaagggccggtttagcatctccagggacgactccaagaacaccctgtatctgcagatgaacagcctgaagaccgaggacaccgctgtgtactactgcgctggcagcaacagggcctttgatatctggggccagggcaccatggtgacagtgtcctccgcctccacaaagggaccttccgtgttccctctggccccttgttcccggtccacctccgaaagcaccgctgctctgggctgcctcgtcaaggactacttccctgagcccgtgaccgtgagctggaactccggcgctctgacaagcggcgtgcataccttccctgccgtgctgcaaagcagcggcctgtatagcctgagcagcgtggtgaccgtgcctagctcctccctgggcaccaaaacctacacctgcaatgtggaccacaagccttccaacaccaaggtggacaagcgggtcgagtccaagtacggccctccttgccctccctgccccgctcccgagtttctgggaggacccagcgtgttcctcttcccccctaagcccaaggacaccctgatgatcagccggacacctgaggtcacctgcgtggtggtggatgtgagccaagaggatcctgaggtccagttcaactggtacgtggacggagtggaggtgcataacgccaagaccaagcctcgggaggagcagttcaactccacctatagggtggtgagcgtgctcacagtgctccaccaggactggctgaacggcaaggagtacaaatgcaaggtgtccaacaagggactccccagcagcatcgaaaagaccatcagcaaggccaaaggccagcccagggaaccccaggtgtacacactgcccccctcccaagaggaaatgaccaagaatcaggtgtccctgacctgcctggtgaaaggcttttaccccagcgacatcgctgtcgagtgggagagcaacggccagcctgagaataactataagaccaccccccccgtgctggatagcgacggatccttcttcctctactcccggctgaccgtggataagtcccggtggcaggagggcaacgtgttcagctgctccgtcatgcacgaggccctgcataaccactacacccagaagtccctgagcctgtccctgggcaagtga

SEQ ID NO: 48 - DNA, 663 bp, Artificial Sequence
  Synthetic Construct
gacatcgtgatgacccagtcccctgattccctggccgtgagcctgggcgaaagggctaccatcaactgcaagtcctcccagagcgtgctgtacgccggcaacaaccggaactatctggcttggtaccagcagaagcccggccagcctcccaagctgctgatcaaccaggctagcaccagggcttccggcgtgcctgataggttcagcggctccggctccggcaccgagtttaccctgatcatctcctccctgcaggccgaggatgtggccatctactactgccagcagtactacacccctcctctggcctttggcggcggcaccaagctggagatcaagaggacagtggccgccccctccgtgttcattttccctccctccgacgagcagctgaagtccggcaccgcctccgtggtgtgcctgctgaacaacttctaccccagggaggccaaggtgcagtggaaggtggacaatgccctgcagagcggcaacagccaggagagcgtcaccgagcaggactccaaagacagcacatacagcctgtccagcaccctgaccctgtccaaggctgactatgagaagcacaaggtgtacgcctgcgaggtgacccaccagggactgagctcccctgtgaccaagtccttcaaccggggagagtgctga

SEQ ID NO: 49 - DNA, 15 bp, Artificial Sequence
  Synthetic Construct
cgggcttggatgaac

SEQ ID NO: 50 - DNA, 57 bp, Artificial Sequence
  Synthetic Construct
cggatcaagaggaaaacagatggcgagaccaccgattacgccgctcccgtgaagggc

SEQ ID NO: 51 - DNA, 21 bp, Artificial Sequence
  Synthetic Construct
agcaacagggcctttgatatc

SEQ ID NO: 52 - DNA, 51 bp, Artificial Sequence
  Synthetic Construct
aagtcctcccagagcgtgctgtacgccggcaacaaccggaactatctggct

SEQ ID NO: 53 - DNA, 21 bp, Artificial Sequence
  Synthetic Construct
caggctagcaccagggcttcc

SEQ ID NO: 54 - DNA, 27 bp, Artificial Sequence
  Synthetic Construct
cagcagtactacacccctcctctggcc

SEQ ID NO: 55 - protein, 444 aa, Artificial Sequence
  Synthetic Construct
EVQLVESGGGLVKPGGSLRLSCAASGLTFERAWMNWVRQAPGKGLEWVGRIKRKTDGETTDYAAPVKGRFSISRDDSKNTLYLQMNSLKTEDTAVYYCAGSNRAFDIWGQGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG